(19) United States
(12) Patent Application Publication
(10) Pub. No.: US 2020/0046603 A1
(43) Pub. Date: Feb. 13, 2020

(54) Vibratory Energy Systems and Methods for Occluded Body Cavities

(71) Applicant: AXIOSONIC, LLC, Effingham, IL (US)

(72) Inventors: Peter M. Bonutti, Manalapan, FL (US); Justin E. Beyers, Effingham, IL (US); Paul A. Westhaver, Newburyport, MA (US)

(21) Appl. No.: 16/659,107

(22) Filed: Oct. 21, 2019

Related U.S. Application Data
(63) Continuation of application No. 13/789,658, filed on Mar. 7, 2013.
(60) Provisional application No. 61/678,097, filed on Jul. 31, 2012.
(60) Provisional application No. 61/677,776, filed on Jul. 31, 2012.
(60) Provisional application No. 61/608,117, filed on Mar. 8, 2012.

(51) Int. Cl. A61H 23/02; A61B 17/3203; A61B 17/22; A61B 17/24; A61H 21/00

(52) U.S. Cl. CPC A61H 23/0245; A61B 17/3203; A61B 17/22012; A61B 2017/320098; A61H 21/00; A61B 17/2202; A61B 17/24

(57) ABSTRACT

Embodiments may include systems and methods for treating an occluded area in a body, accessing cavities or passages of the body, or reducing pathologic material in the body. Embodiments may be configured to apply vibratory energy to pathologic material in a treatment area of a body. A handle connected to an energy source may be configured to provide an energy signal. A transducer may be configured to receive the energy signal. An effector may be operatively coupled to the transducer. The effector may have a proximal end connected to the handle and a distal portion configured to apply vibratory energy to pathologic material. A cannula may have a longitudinal passage to receive at least a portion of the effector and/or be configured to expose at least the distal portion of the effector to the pathologic material or the treatment area.

### CROSS-REFERENCE

This application claims the benefit under 35 U.S. § 119(e) of U.S. Provisional Application No. 61/608,117 filed Mar. 8, 2012, titled “ULTRASONIC DEVICES AND METHODS FOR BODY CAVITIES AND PASSAGES”, U.S. Provisional Application No. 61/678,097 filed Jul. 31, 2012, titled “SYSTEMS AND METHODS FOR ULTRASONIC SINUS TREATMENT”, and U.S. Provisional Application No. 61/677,776 filed Jul. 31, 2012, titled “METHODS AND SYSTEMS FOR EXTRACTING TISSUE USING A HANDHELD MEDICAL DEVICE”, which are hereby incorporated by reference in their entirety.

### FIELD

Embodiments of the present disclosure relate to devices and methods for ablation, removal, reduction, disruption, modification, and/or killing of pathologic or other materials in or on a body. Further embodiments relate to devices and methods to access and/or treat a portion of the body, for example cavities and passages of the body. Embodiments also relate to devices and methods configured to apply and/or control energy, for example vibratory energy. Additional embodiments may relate to devices and methods configured to extract pathologic material or other materials from the body.

### BACKGROUND

Pathologic materials and/or an occluded passage or cavity of a body may cause discomfort or pain and may ultimately require medical treatment. For example, sinus headaches are often caused by increased pressure in the sinuses, for example the paranasal sinuses including the frontal, maxillary, ethmoid, and sphenoid sinuses. This pressure may be caused by failure of fluids to properly drain into the nasal passage. Sometimes the ducts, by which the sinuses drain, become partially or fully occluded thereby preventing proper drainage. The occlusion may be due to an infection, nasal polyps, or another medical condition. Traditional treatment methods may include leaving the condition untreated, using pain and anti-inflammatory drugs, or performing surgery, such as, functional endoscopic sinus surgery (FESS) or balloon sinuplasty. However, traditional techniques have numerous shortcomings.

As a surgical approach to establishing normal sinus drainage, FESS removes or corrects diseased tissues obstructing the sinus. This typically involves enlarging the maxillary cavity by performing an uncinectomy using the “swing door” technique. The uncinate, part of the medial wall of maxillary sinus, is swung medially and then severed at its lateral attachment. This is followed by a submucosal removal of the horizontal process of the uncinate and trimming of the mucosa. Depending on the presence of fungal mucin within the sinus, the cavity may be enlarged superiorly to orbital floor and posteriorly to posterior fontanelle. FESS is a drastic surgical approach resulting in unnecessary tissue loss and post-operative recovery.

In a less invasive technique, balloon sinuplasty includes insertion of a guide cannula through the nostril to gain access to the sinus cavity. A guide wire is then introduced into the occluded sinus through the guide cannula. A balloon cannula is then introduced into the sinus cavity over the guide wire and positioned in the occluded cavity. The balloon is inflated to open the narrowed or occluded cavity then deflated and removed. An irrigation cannula is then advanced over the guide wire. Finally, the irrigation cannula is removed from the sinus to allow the sinus cavity to drain. However, balloon sinuplasty devices do not fit into every sinus cavity, potentially cause excess trauma, are unable to treat all sinus related conditions (i.e. nasal polyps), and are susceptible to occlusion recurrence.

There exists a need for devices and methods for treating pathologic materials and/or occluded cavities and passages in the body, for example the sinuses. Furthermore, minimally invasive devices and methods may allow for less post-operative pain and discomfort, shorter recovery times, higher accuracy, and less tissue damage than traditional techniques.

### SUMMARY

Embodiments may include devices, systems, and methods for treating an occluded area in a body, accessing cavities and/or passages of the body, and/or reducing pathologic material in the body. For example, systems may be configured to apply vibratory energy to pathologic material in a treatment area of a body. Systems may include a handle connected to an energy source configured to provide an energy signal, a piezoelectric transducer configured to receive the energy signal, an effector operatively coupled to the transducer and/or having a proximal end connected to the handle and a distal portion configured to apply vibratory energy to pathologic material, and/or a cannula having a longitudinal passage to receive at least a portion of the effector and being configured to expose at least the distal portion of the effector to the pathologic material. The transducer may be configured to transfer vibratory energy through the effector to the pathologic material. The cannula may shield at least a portion of the body from vibratory energy.

In another embodiment, systems may include a handle connected to an energy source configured to provide an energy signal, a piezoelectric transducer configured to receive the energy signal, and/or an effector operatively coupled to the transducer and/or having a proximal end connected to the handle and a distal portion configured to apply vibratory energy to pathologic material. The transducer may be configured to transfer vibratory energy through the effector.

Further embodiments may include methods for using a vibratory energy system in a treatment area of a body. The methods may include a handpiece having a piezoelectric transducer operatively connected to a effector, providing a cannula having a longitudinal passage configured to receive the effector and being configured to shield a portion of the body from the effector, positioning the effector and cannula in the treatment area with at least a distal portion of the effector extending beyond the cannula, and/or applying energy to the transducer to vibrate the effector. The effector may be configured to transfer vibratory energy to and/or apply surface erosion to the treatment area.

Embodiments may also include methods comprising the acts of providing a handpiece including a handle and an effector having a flexible elongated portion and a distal portion, forming a hole through an imperforate tissue of the body, passing at least the distal portion of the effector through the hole and into the treatment area, and/or applying vibratory energy to the treatment area with at least the distal portion of the effector.

In additional embodiments, methods may include of controlling a vibratory energy device, the method comprising, applying a first tuning signal in a first desired frequency range to a vibratory energy device, receiving a returned parameter from the vibratory energy device, detecting two or more operating frequencies from the returned parameter, and modulating a drive signal to the vibratory energy device between detected operating frequencies in a pattern until two are more detected frequencies have been applied.

Further embodiments may include a system for applying vibratory energy to pathologic material in a treatment area of a body. The system may comprise a medical device positionable external to a portion of the body and an effector including a piezoelectric transducer disposed in the medical device. The effector may be configured to apply energy to a portion of the body.

Additional embodiments of the present disclosure are described throughout including the drawings.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 illustrates an embodiment of the present disclosure, for example a handpiece;

FIG. 2 illustrates an embodiment of FIG. 2, for example, a handpiece with an alternative distal portion;

FIG. 3 illustrates an embodiment of FIG. 2, for example, a handpiece with another alternative distal portion;

FIG. 4 illustrates an embodiment, for example, positionable with respect to a maxillary sinus;

FIG. 5 illustrates an embodiment, for example, positionable with respect to a frontal sinus;

FIG. 6 illustrates an embodiment, for example, including penetration of tissue;

FIG. 7 illustrates an embodiment, for example, including an externally positionable effector;

FIG. 8 illustrates an embodiment, for example, including a cannula and effector with a distal portion;

FIG. 9 illustrates an embodiment, for example, including an alternate distal portion in an extended configuration;

FIG. 10 illustrates an embodiment of FIG. 9, for example, including a contracted configuration;

FIG. 11 illustrates an embodiment, for example, including pathologic materials;

FIG. 12 illustrates a side view of FIGS. 4, 5, 6, and 11;

FIG. 13 illustrates an isometric view of an embodiment, for example a handpiece;

FIG. 14 illustrates an embodiment, for example, including a transducer;

FIG. 15 illustrates an embodiment, for example, including a transformer;

FIG. 16 illustrates a section view of FIG. 15, for example, including a transformer portion;

FIG. 17 illustrates a proximal portion view of FIG. 15;

FIG. 18 illustrates a closer view of a distal portion of FIG. 15;

FIG. 19 illustrates an embodiment, for example, including a removed handle portion and crystals of a transducer;

FIG. 20 illustrates an embodiment, for example, including a cannula assembly that may be configured to receive a handpiece;

FIG. 21 illustrates an embodiment, for example, including a step and/or abrasive features;

FIG. 22 illustrates a closer view of FIG. 21;

FIG. 23 illustrates an embodiment, for example, including a nested cone feature;

FIG. 24 illustrates an embodiment, for example, including a helix feature;

FIG. 25 illustrates an embodiment, for example, including a dimple feature;

FIG. 26 illustrates an embodiment, for example, including a flute feature;

FIG. 27 illustrates an embodiment, for example, including a chevron feature;

FIG. 28 illustrates an embodiment, for example, including a hole feature;

FIG. 29 illustrates an embodiment, for example, including a bump feature;

FIG. 30 illustrates an embodiment, for example, including an attachment;

FIG. 31 illustrates an embodiment, for example, including a handpiece that may include a cannula or sheath with an offset and/or angled effector;

FIG. 32 illustrates an embodiment, for example, including a chamber connecting a handpiece and cannula assembly;

FIG. 33 illustrates an embodiment, for example, including an alternative handpiece;

FIG. 34 illustrates an embodiment, for example, including one or more effector that branches or combines into filaments;

FIG. 35 illustrates an embodiment, for example, including a cannula configured to receive filaments;

FIG. 36 illustrates an embodiment, for example, including a mass on a portion of an effector;

FIG. 37 illustrates an embodiment, for example, including an effector with a kink;

FIG. 38 illustrates an embodiment, for example, including a concentrated mass at a portion along an effector;

FIG. 39 illustrates an embodiment, for example, including a handpiece with a channel and port;

FIG. 40 illustrates an embodiment, for example, including a projector assembly with an effector and cannula;

FIG. 41 illustrates a section view, for example, including a projector assembly;

FIG. 42 illustrates an embodiment, for example, including an effector with a plurality of projectors;

FIG. 43 illustrates an embodiment, for example, including dimensional options and a plurality of effectors that may produce two or more phases in connection with a transducer;

FIG. 44 illustrates an embodiment, for example, including wave position examples for a plurality of effector tips;

FIG. 45 illustrates an embodiment, for example, including an effector with a torus or funnel shape;

FIG. 46 illustrates an embodiment, for example, including an effector that may be configured to ablate or coagulate a passage;

FIG. 47 illustrates an embodiment, for example, including an alternate effector in a passage;

FIG. 48 illustrates an embodiment, for example, including an effector including elements;

FIG. 49 illustrates an embodiment, for example, including a tissue of varying surface dimensions;

FIG. 50 illustrates an embodiment of FIG. 48, for example, including an effector conforming to and/or providing controlled energy and/or pressure along a body portion;

FIG. 51 illustrates an embodiment, for example, including characteristics of energy provided to and/or transferred through an effector;

FIG. 52 illustrates an embodiment, for example, including a guidance unit;

FIG. 53 illustrates an embodiment, for example, including a contracted configuration of an effector a foil and elements;

FIG. 54 illustrates a closer view of FIG. 53, for example, including elements;

FIG. 55 illustrates an embodiment of FIG. 53, for example, including an expanded configuration;

FIG. 56 illustrates an embodiment, for example, including dimensional options and a plurality of transformers;

FIG. 57 illustrates an embodiment, for example, including a streaming wire and/or pump actuator;

FIG. 58 illustrates a closer view of FIG. 57, for example, including a step of an effector;

FIG. 59 illustrates an embodiment, for example, including movement options of an effector;

FIG. 60 illustrates an embodiment, for example, including an effector including a portion that may be configured to access an anatomic curvature;

FIG. 61 illustrates an embodiment, for example, including an alternative distal portion of an effector;

FIG. 62 illustrates an embodiment, for example, including another alternative distal portion of an effector;

FIG. 63 illustrates an embodiment, for example, including a handpiece that may be configured to receive another medical instrument;

FIG. 64 illustrates an embodiment, for example, including a medical instrument configured to receive an effector or another medical instrument;

FIG. 65 illustrates an embodiment, for example, including a transducer with a first impedance and/or stiffness and an effector with a second impedance and/or stiffness;

FIG. 66 illustrates an embodiment, for example, including a transducer with an alternate attachment;

FIG. 67 illustrates a cross-section view of an embodiment, for example, including a transducer with a grasper and a cannula;

FIG. 68 illustrates a component view of FIG. 67, for example, including a grasper;

FIG. 69 illustrates a schematic of FIG. 67, for example, including a hypothetical application of force;

FIG. 70 illustrates a side view of an embodiment of FIG. 67, for example, including an effector;

FIG. 71 illustrates an embodiment, for example, including an alternate grasper;

FIG. 72 illustrates an alternate view of FIG. 71;

FIG. 73 illustrates a section view of FIG. 72;

FIG. 74 illustrates an embodiment, for example, including an alternate effector;

FIG. 75 illustrates an embodiment, for example, including a transducer and another alternate effector;

FIG. 76 illustrates an embodiment, for example, including an alternate handpiece;

FIG. 77 illustrates an embodiment, for example, including an effector in a contracted configuration;

FIG. 78 illustrates an embodiment, for example, including an effector in an expanded configuration;

FIG. 79 illustrates an embodiment, for example, including an alternate cannula having a sensor with elements;

FIG. 80 illustrates an embodiment, for example, including an element;

FIG. 81 illustrates an embodiment, for example, including a flexible effector and/or flexable cannula;

FIG. 82 illustrates a diagram of the embodiment of FIG. 79, for example, including hypothetical sensor outputs;

FIG. 83 illustrates an alternate embodiment of FIG. 79, for example, including a light;

FIG. 84 illustrates another alternate embodiment of FIG. 83;

FIG. 85 illustrates another alternate embodiment of FIG. 83;

FIG. 86 illustrates a schematic of FIGS. 83-85, for example, including a circuit powered by induced piezoelectric ceramic;

FIG. 87 illustrates an embodiment, for example, including a cannula with embedded illumination;

FIG. 88 illustrates an embodiment, for example, including an illuminated cannula;

FIG. 89 illustrates an embodiment, for example, including an alternate illuminated cannula;

FIG. 90 illustrates an embodiment, for example, including handpiece with a diffuser and light array;

FIG. 91 illustrates an embodiment, for example frequency hopping;

FIG. 92 illustrates an embodiment, for example, including cyclical frequency hopping;

FIG. 93 illustrates a diagram, for example, including a hypothetical mixed-frequency signal;

FIG. 94 illustrates a diagram, for example, including a hypothetical amplitude modulation;

FIG. 95 illustrates an embodiment, for example, a balloon in an expandable cannula;

FIG. 96 illustrates an embodiment, for example, including a balloon outside a cannula;

FIG. 97 illustrates an embodiment, for example, including a projected and directional energy application;

FIG. 98 illustrates an alternate view of FIG. 97, for example, including rotation and/or articulation;

FIG. 99 illustrates an embodiment, for example including an alternate projector and/or a projector retainer;

FIG. 100 illustrates an alternate view of FIG. 99;

FIG. 101 illustrates an embodiment, for example, including alternate projector surfaces;

FIG. 102 illustrates an embodiment, for example, including alternate projector surfaces;

FIG. 103 illustrates an embodiment, for example, including alternate projector surfaces;

FIG. 104 illustrates an embodiment, for example, including alternate projector surfaces;

FIG. 105 illustrates an embodiment, for example, including a capsule projector;

FIG. 106 illustrates a section view of FIG. 105;

FIG. 107 illustrates an embodiment, for example, an alternate capsule projector;

FIG. 108 illustrates an embodiment of FIG. 107, for example positioned in a cavity;

FIG. 109 illustrates an embodiment, for example, including a capsule projector with a light;

FIG. 110 illustrates an alternate view of FIG. 109;

FIG. 111 illustrates an embodiment, for example, including a ring projector with reinforcement;

FIG. 112 illustrates an embodiment, for example, including a ring projector with alternate reinforcement;

FIG. 113 illustrates an end view of FIG. 112;

FIG. 114 illustrates an embodiment, for example, including a ring projector with optional dimensions;

FIG. 115 illustrates an embodiment, for example, including a ring projector expanding;

FIG. 116 illustrates an embodiment, for example, including a ring projector contracting;

FIG. 117 illustrates an embodiment, for example, including a projector in a medium;

FIG. 118 illustrates an embodiment, for example, including a hypothetical contraction beam pattern with respect to a ring projector;

FIG. 119 illustrates an embodiment, for example, a hypothetical expansion beam pattern with respect to a ring projector;

FIG. 119 illustrates an embodiment, for example, another hypothetical expansion beam pattern with respect to a ring projector;

FIG. 120 illustrates an embodiment, for example, an alternate encapsulation of a projector;

FIG. 121 illustrates a component view of FIG. 120;

FIG. 122 illustrates a section view of FIG. 121;

FIG. 123 illustrates another section view of FIG. 121;

FIG. 124 illustrates a section view of FIG. 120;

FIG. 125 illustrates an embodiment, for example, including a projector configured to an anatomic curvature of a maxillary sinus cavity;

FIG. 126 illustrates an embodiment, for example, including a ring projector applying energy to and/or through tissues and pathologic materials;

FIG. 127 illustrates an embodiment, for example, including application of energy to a cell of a pathologic material;

FIG. 128 illustrates an embodiment, for example, including an externally positionable transducer;

FIG. 129 illustrates a closer view of an embodiment of FIG. 128;

FIG. 130 illustrates an embodiment, for example, including an alternate capsule projector with a hemispherical bond and beam pattern;

FIG. 131 illustrates an embodiment, for example, including application of energy to a pathologic material and/or through a tissue structure;

FIG. 132 illustrates an embodiment, for example, including projector connections configured to reduce constraint and/or mass;

FIG. 133 illustrates a closer view of FIG. 132;

FIG. 134 illustrates a diagram of an embodiment, for example, including hypothetical voltage and pressure characteristics;

FIG. 135 illustrates another diagram of an embodiment, for example, including hypothetical voltage and pressure characteristics;

FIG. 136 illustrates another diagram of an embodiment, for example, including hypothetical voltage, pressure, and frequency characteristics;

FIG. 137 illustrates another diagram of an embodiment, for example, including hypothetical voltage and voltage change characteristics;

FIG. 138 illustrates another diagram of an embodiment, for example, including voltage characteristics;

FIG. 139 illustrates an embodiment, for example, including transducer including a torsionally transformable configuration;

FIG. 140 illustrates multiple views of an embodiment, for example, including a distal portion of an effector;

FIG. 141 illustrates multiple views of an embodiment, for example, including an alternate distal portion of an effector;

FIG. 142 illustrates an embodiment, for example, including a wrap;

FIG. 143 illustrates an embodiment, for example, including a brace; and

FIG. 144 illustrates an embodiment, for example, including a continuous passive motion device.

### DETAILED DESCRIPTION

Embodiments of the present disclosure may relate to devices and methods for accessing, penetrating, ablating, removing, reducing, modifying, killing, and/or resisting formation of pathologic material in any portion of a body. The body portion may be an entirely or partially occluded cavity of the body, for example a paranasal sinus. Pathologic material may include any materials that may obstruct a portion of the body, cause infection, pain, or discomfort, inhibit healthy tissue function, or are otherwise undesirable. Pathologic material may include any materials that entirely or partially occlude a passages and/or cavities of the body, for example sinuses, ducts, and vasculature. Pathologic material may include pus, mucus, biofilm, bacteria, plaque, polyps, cysts, tumors, calcifications, calcific deposits, scarring, collapsed or diseased tissue, fibrous materials, hardened masses, infectious materials, and/or any other tissue or material that is unhealthy, obstructive, infectious, and/or undesirable. Pathologic material may include a tissue type or portion that needs to be modified, changed, or altered relative to another type or portion of tissue. The present disclosure further relates vibratory energy devices and methods configured to access and/or treat a portion of the body. The present disclosure also relates to control systems and methods, for example, related to vibratory energy, target surface erosion, and/or effector to tissue surface contact trajectory.

Embodiments may include application of energy to biofilm. Biofilm may include any combination of human tissue, infectious organisms such as living bacteria and/or fungus, and/or material produced by the infectious organisms. Considering that biofilm may contain living organisms and cells, the response of the individual cells to exposure of energy (i.e. time varying acoustic fields) may make biofilm susceptible to exposure to acoustic energy.

For the purposes of this disclosure, the terms related to sound, ultrasound, acoustics, vibration, wave mechanics, and sonar are defined herein. Acoustics includes properties or qualities that determine the transmission of waves. Acoustics may be with respect to matter, for example gases, liquids, and/or solids. Acoustics may include vibration, sound, ultrasound, and infrasound. Acoustic energy may include a disturbance of energy that comes through matter as a wave, for example movement of particles in a medium in response to a mechanical wave. Sound is generally regarded as vibration in the frequency range of hearing of a human, for example above about 20 Hz and below 20,000 Hz. Ultrasound may include acoustics at or above 20,000 Hz but many ultrasonic devices vibrate sub-harmonics that exist in the sound frequency range. Infrasound is generally referred to vibration that may not be detected by the human ear and has a frequency of about 20 Hz and below. Vibration as disclosed herein may exist in any or all frequency ranges.

Embodiments may be configured for vibratory energy of any frequency, intensity, and/or pressure level. Vibratory energy may be applied to inhibit the reproduction and/or to kill pathologic material in the body, for example in the paranasal sinuses. Sound may be lethal to living things. Sound including time varying pressure waves in a medium may be lethal or harmful to many organisms if the sound is of sufficient intensity. Sound in frequencies above 20 kHz may kill organisms. For example, bacteria may be killed with ultrasound between levels of 156 to 164 decibels. Infections exposed to transdermal sound and ultrasound at 10 kHz to 10 Mhz may result in relief of symptoms.

The sinus cavity of a person suffering chronic rhinitis may be infected with numerous bacteria or fungal species. For example, biofilm may integrate into the mucus layer of the nasal passage and form stubborn and durable saccharine binding agents. Biofilm also may integrate beneath and among the endothelial cells of the nasal passages making it difficult for the normal immune system to overcome the infection. Therapeutic substances may be used to kill the infections and wash the fungi and infection away from the native tissue. Embodiments may be configured to reduce or kill biofilms while preserving healthy tissues, for example endothelial cells and cilia. Alternative, embodiments may be configured to modify biofilm to a more desirable state.

The nasal cavity of a human being has many passages, cavities, and folds of tissue covering passages and cavities. The tissue may include mucus layers, cavities filled with air, cavities filled with fluids, blind cavities with no passageways to the exterior of the body, and passages that lead against gravity to the exterior of the body. Embodiments may include instruments to access any or all of the crevices with the nasal cavity structure without significant trauma.

Embodiments disclosed herein may be configured to apply energy, for example vibratory energy, to any portion of a body. Embodiments may include an emitter. Emitters may include a transducer, magnet, electromagnet, radiofrequency (RF) device, or any energy device disclosed herein or otherwise configured to transmit energy. Emitters may be placed with respect to any portion of the body to allow transmission of energy to a treatment area of the body.

For the purposes of this disclosure, a transducer may include any device or assembly that converts one form of energy to another form of energy. A transducer may also include any device or assembly that converts variations in a physical quantity, such as pressure or brightness, into an electrical signal, or vice versa. Energy types may include electrical, mechanical, vibratory, ultrasonic, magnetic, electromagnetic, optical (i.e. light), chemical, electrostatic, acoustic, radioacoustic, thermal energy, and/or any other energy type disclosed herein. Embodiments may include many components of a vibratory system. A transducer may include a single component (i.e. crystal) or an assembly of piezoelectric elements (i.e. quartz or PZT). The piezoelectric elements may be joined to electrodes that may include any conductor, for example copper, silver, steel, and/or any other conductor disclosed herein. For the purposes of this disclosure, piezoelectric elements may be interchangeably referred to as crystals or “piezos”. The electrodes and crystals may be clamped with a structure that holds the crystals and the electrodes together. The transducer may include the assembly or a single component (i.e. a crystal) capable of transducing electrical energy to mechanical energy through the piezoelectric effect. A transducer may also be considered a trap or a container for a resonating vibrational system. The transducer may be configured to transmit energy, for example ultrasonic energy. The transducer may be a piezoelectric transducer including a stack or array of piezoelectric crystals that may be operatively connected to the effector. All or any portion of the effector may be elongated, flexible, curved, and/or bendable. One or a plurality of effectors may include, for example, a wire, cable, and/or rod shape. The effector may have a proximal portion connected to the handpiece and a distal portion positionable in a portion of the body, for example a passage or cavity. The effector may be cannulated to allow for damping, lubrication, medium supply, irrigation and/or suction.

The transducer may have any number of configurations. The transducer may be located at any portion of the system, for example, in the handpiece or in the effector. The transducer may be in any portion of the effector, for example a distal portion. The transducer may be configured to transmit energy along a length of the effector and/or to a portion of the body. In another embodiment, the distal portion of the effector may also include a transducer configured to transmit energy from the distal portion to a portion of the body. The transducer may be configured for percutaneous access into the body through the skin or another passage or for transcutaneous transmission into the body from outside the body. Embodiments may be configured to affect tissue in direct contact, near, or at a distance from the effector.

For the purposes of this disclosure, a horn may include a component of a vibratory energy system (i.e. resonant Langevin type), which may include a trumpet, cylinder, or any other shape. A horn may have features that exploit a positionally varying waveform contained within or passing through the horn. The horn may, for example, be changed in diameter at a node of resonant vibration to alter the strain wave local amplitude. The horn may be of any length. If the length of the horn is a multiple of the wavelengths of vibration, the horn may act as a wave guide, for example a long horn primarily carrying vibrational energy with minimal focusing or diffusing. For the purposes of this disclosure, transformer refers to features or components that alter, convert, combine, or split the modes of vibration. For example, a longitudinal wave may be partially converted into a transverse or torsional wave of the same or similar frequency. The feature or component employed to accomplish this is the transformer. The term effector refers to a device or component that affects another material, for example a body portion or pathologic material. Depending on the configuration, the effector may be used herein to refer to a horn, a wave guide, and/or a projector. A projector is a transducer that may receive electrical energy from a generator, transduce it into mechanical energy, and/or acoustically transfers it into the surrounding medium, for example in a spatially and/or temporally varying stress field. The shape of the field is referred to herein as a beam pattern. A beam pattern may be, for example, a uniform spherical and/or growing stress field. In the audio sound frequency realm, a projector may include a speaker. In the broader realm of acoustics, a projector may include characteristics beyond beam pattern, medium, and frequency.

Energy may also include or be used in conjunction with vibratory energy (i.e. ultrasonic energy), thermal energy, resistive heating, radiofrequency (RF), microwave, laser, magnetic, electromagnetic, electro shockwave therapy, and/or plasma energy (hot or cold). Energy may be applied when the effector is positioned in the treatment area. Embodiments disclosed herein may use any operating parameters, for example any power, frequency, current, or voltage. Exemplary vibratory energy operating frequencies may include ranges between any of 0 kHz, 0.03 kHz, 1 kHz, 10 kHz, 20 kHz, 40 kHz, 60 kHz, 80 kHz, 100 kHz, 120 kHz, 160 kHz, 180 kHz, 200 kHz and/or 500 kHz. The power or operating frequency may be adjusted during operation of the handpiece and/or depending on the material characteristics of pathologic material or body tissue. Embodiments may be configured to apply two or more energy types and/or be used any other energy device disclosed herein or known in the art.

Energy may be applied based on a targeted treatment area. Embodiments may be configured to apply energy based on the material characteristics of the treatment area, for example the material characteristics of body tissue, pathologic material, and/or therapeutic substances. Material characteristics may include mechanical resonance, electrical resonance, density, modulus of elasticity, and/or any thermal, chemical, and/or molecular properties. Material characteristics may be with respect to all or any portion of any embodiment herein including any individual cells, sub-cellular structures, cell types. Energy may be applied based on material type (i.e. soft tissue vs. hard tissue and/or natural vs. synthetic), viability (i.e. viable vs. non-viable), and/or depth (i.e. superficial tissue vs. deep tissue). Embodiments may be configured to modify material characteristics of targeted areas or materials. Embodiments may be configured to change and/or apply energy based on the shape or size of pathologic materials or a body tissue. Further embodiments may be configured to stimulate growth and/or healing of soft or hard body tissues (i.e. bone) and/or release hormones, white blood cells, and/or enzymes. Additional embodiments may be configured to erode and/or debride targeted areas or materials.

Embodiments may include and/or be positioned with minimally invasive and/or access devices, for example a cannula. The cannula may include a sheath, for example, to at least partially protect or shield portions of the body from undesired contact with or energy from the effector. The cannula may be cannulated and/or include a port or valve, for example, to allow for irrigation, suction, and/or transfer of materials. Embodiments may include an inflow portion and/or may be connected to a suction device. The effector and/or cannula may be configured to focus energy to the treatment area.

Embodiments may be configured to access any treatment site, for example a portion of the body. Embodiments may be configured to transfer objects and/or materials into and/or from the treatment site. Embodiments may utilize a natural body passage or create a passage. The passage may be created through soft or hard tissue, for example bone. The passage may be created with the distal portion of the handpiece and/or a cutting instrument, for example a drill, a trocar or cannula, or energy (i.e. ultrasonic) transmitter. The drill may include a cannulated drill to allow access through a passage therein. The effector may include a balloon configured to expand the passage and/or position the distal portion of the effector, for example, while vibratory energy is applied along the effector. Embodiments may be partially or entirely flexible, curved, non-linear, bendable, and/or may have shape memory properties or materials, which may allow all or any portion of the system to change in shape. A change in shape may include a change in angle, which may range between about 0-180 degrees. Use of a shape memory material may allow the angle to vary within a range of about 0-180 degrees with a change in temperature and/or by the application of heat. Embodiments may be shaped and/or positioned to access a curved or natural anatomic path through the body. Embodiments may be used in conjunction with any devices or methods disclosed in U.S. Pat. No. 6,814,715, titled “Expandable Cannula” and U.S. Patent Application Publication Nos. 2011/0202123, titled “Anatomic Needle System”, 2007/0088362, titled “Apparatus and Methods for Surgery”, and 2011/0224539, titled “Methods for Positioning an Ultrasonic Catheter” and U.S. patent application Ser. No. 13/683,847, titled “Expandable Access Systems and Methods”, all of which are hereby incorporated by reference in their entirety.

The methods and devices disclosed herein may be used in conjunction with any medical procedure. Embodiments may be used before, during, or after a procedure, for example to create or increase a passage in the body and/or to remove pathologic material. Treatment areas may include any cavity, vessel, duct, passage, joint, bone, muscle, ligament, tendon, cartilage, capsule, organ, skin, nerve, vessel, or other body parts. Embodiments may be used for applications related to biliary ducts, bronchi (i.e. cystic fibrosis), kidney stones, bile ducts, sinus ducts, bone cavities, the vasculature, and any other site in the body that contain a passage or cavity. As further examples, embodiments herein may be used in or in conjunction with other medical instruments during sinuplasty, lithotripsy, intervertebral disc surgery, kyphoplasty, knee surgery, hip surgery, organ transplant surgery, bariatric surgery, spinal surgery, anterior cruciate ligament (ACL) surgery, tendon-ligament surgery, rotator cuff surgery, capsule repair surgery, fractured bone surgery, pelvic fracture surgery, avulsion fragment surgery, shoulder surgery, hernia repair surgery, and surgery of an intrasubstance ligament tear, annulus fibrosis, fascia lata, or flexor tendons. Treatment areas include the ear, prostate, biliary ducts, bronchi (i.e. cystic fibrosis), kidney stones, bile ducts, sinuses (i.e. sinusitis), small or large intestines (i.e. diverticulitis), bone cavities, and/or vasculature. Embodiments may be used in any portion of the body that may benefit from an improved passage or cavity or reduction of pathologic material. Embodiment may be used in any medical application or body portion disclosed herein, disclosed in the incorporated references, or known in the art.

Embodiments herein may include any biocompatible materials or other materials suitable for medical use. Embodiments may include portions and/or combinations of metals and polymers. Embodiments may include shape memory alloys (SMA) and/or shape memory polymers (SMP). Examples of amorphous polymers are polycarbonate (LEXAN), polystyrene, polysulfone (ULDALL), and acrylics polycarbonate (ABS and styrenes). Examples of semi-crystalline polymers include acetyl (DELRIN), nylon, polyester, polyethylene, polyether ether ketone, polypropylene, polyvinylchloride (PVC), and Caprolactam. Biodegradable semi-crystalline polymers may include polylactic acid and polyglycolic acid. Copolymers of PGA and PLA may also be used. Poly-1-lactide (PLLA) or other forms of PLA may also be used. Other polymers which may be used with the present disclosure, either as a thermoplastic or non-thermoplastic, are polyethylene glycol (PEG)-copolymers and D,L-lactide-co-glycolide polyesters. Some semi-crystalline materials are particularly suitable for surgical bonding and/or staking, especially vibratory bonding and staking. Examples of such materials include PAEK (polyaryletherketone), including PEEK (polyetheretherketone) and PEKK (polyetherketoneketone). Metals include stainless steel, 17-4 steel, shape metal alloys, tantalum, porous tantalum, titanium, and cobalt-chrome alloys. Shape memory alloys may include nitinol (nikel-titanium). Shape memory polymers may include PEEK, PMMA, and thermoset polymers. Thermoset polymers may include polyurethanes, polyethylene terephthalate (PET), polyethyleneoxide (PEO), block copolymers containing polystyrene and poly(1,4-butadiene), and ABA triblock copolymers, for example including poly(2-methyl-2-oxazoline) and polytetrahydrofuran. Ceramic materials (i.e. implants) may include silicon nitride, alumina (aluminum oxide), and zircon (zirconium dioxide). Embodiments may include polycarbonate, resin-glass composite, carbon fiber, Kevlar, polyethylene (i.e. HDPE), silicone, urethane, and/or Teflon. Embodiments may include materials configured to resist growth of bacteria and/or biofilm, for example silicon nitride.

Embodiments may be any of biocompatible, degradable, biodegradable, bioerodible, bioabsorbable, mechanically expandable, hydrophilic, bendable, deformable, malleable, riveting, threaded, toggling, barded, bubbled, laminated, coated, blocking, pneumatic, one-piece, multi-component, solid, hollow, polygon-shaped, pointed, self-introducing, mesh, segmented, tubular, braided, suture material, elastic (i.e. rubber, silicone, or elastic materials), and combinations thereof. Furthermore, embodiments may include any of a metallic material, polymeric material, ceramic material, composite material, body tissue, synthetic tissue, hydrophilic material, expandable material, compressible material, bondable material, and combinations thereof. Embodiments may also include polymethyl methacrylate (PMMA or “bone cement”), glue, adhesive, grouting agents, acrylic materials, and combinations thereof.

All or any portion of the system may be shaped, assembled, bonded, and/or positioned with energy, magnetic field, chemical reaction, mechanical interlocking, application of force, adhesives, and/or solvents. Embodiments may assembled and/or secured relative to the body by attaching, engaging, connecting, binding, adhering, and/or fastening one or more materials through resistive heating, mechanical interlocking, application of force, application of grouting agents (i.e. bone cement), adhesives and/or solvents, spraying, radiofrequency, vibratory energy (i.e. ultrasound), microwave, laser, magnetic, electromagnetic, electro shockwave therapy, plasma energy (i.e. hot or cold), and any other method described herein.

Embodiments may be configured to be used in conjunction with other medical instruments or implants. Embodiments may be configured to position and/or prepare the treatment site for another medical instrument or implant. Effector 110 may be configured to transfer energy to another medical instrument or implant. Medical instruments or implants may direct or focus energy from effector 110. Instruments may include any dilator, trocar, introducer, imaging device, sinuplasty device, or any other device or material disclosed herein. Implants may include a coronary artery stent, vascular stent, peripheral vascular stent, urinary tract stent, and/or urethral stent. Implants may include a partial or total knee replacement, hip replacement, shoulder replacement, bone fastener, etc. Objects may include an organ, partial organ grafts, tissue graft material (i.e. autogenic, allogenic, xenogenic, or synthetic), collagen, a malleable implant like a sponge, mesh, bag/sac/pouch, collagen, or gelatin, or a rigid implant made of metal (i.e. porous or nonporous), polymer, composite, or ceramic. Other implants include breast implants, biodegradable plates, porcine or bovine patches, metallic fasteners, compliant bearing for medial compartment of the knee, nucleus pulposus prosthetics, stents, fasteners, sutures, suture anchors, tissue grafts, or tissue scaffolds.

Tissue scaffolds may include any biologic, synthetic, biodegradable, collagen, polymeric and/or biocompatible scaffold. The scaffold may include a collagen matrix configured to receive viable cells of any type. The matrix may be utilized as a support structure for cells. Different types of cells may be placed at various locations in the matrix. The matrix may be positioned relative to and/or any portion of a patient's body, for example all or any portion of a heart, blood vessel, brain, intestine, stomach, adrenal gland, liver, pancreas, bone, skeleton, spinal cord, or any other organ or any soft or hard tissue. The cell types may include progenitor cells which differentiate and proliferate to form cells having desired characteristics, stromal cells which relate to foundation supporting tissue, and mesenchymal cells which relate to connective tissues, blood and blood vessels, and other systems. Fibroblasts may be used in the production of connective tissues. Osteoblasts may be used in the production of hard tissue (i.e. bone). Myoblasts may be used in the production of muscle. Specific cells may be used to provide for growth of tissue having a function associated with the cell, which may include reticular cells, smooth muscle cells, chondrocytes, retinal cells, endothelial cells, fetal cells, stem cells, embryonic cells, adult cells, enzymes, proteins, and/or other cells disclosed herein or known in the art. Once the viable cells have been positioned on the matrix, the result is a replacement tissue (i.e. an organ). Embodiments of the present disclosure may include the additional devices and methods disclosed in U.S. Pat. No. 7,299,805, titled “Scaffold and Method for Implanting Cells”, which is hereby incorporated by reference in its entirety. Embodiments may also include biofilm that is sterilized to allow sterile biofilm to be used as an adhesive for scaffolds or used as a drug release agent.

Embodiments may include and/or provide therapeutic substances, for example, to promote healing and/or reduce pathologic material. Embodiments may be configured to apply and/or transfer therapeutic substances to and/or from the body. Therapeutic substances may include any fluid, gas, or other material that may be beneficial to performing a medical procedure, treating a medical condition, reducing pathologic material, or to promoting healthy tissue. Therapeutic substances could include pharmaceutical agents, antibiotics, astringents, bactericides, hydroxyapatite, anti-inflammatory agents, steroids, antibiotics, analgesic agents, chemotherapeutic agents, bone morphogenetic protein (BMP), demineralized bone matrix, collagen, growth promoting materials or surfaces (i.e. porous materials or pores), growth factors, autogenetic bone marrow, progenitor cells, calcium sulfate, immunosuppressants, fibrin, osteoinductive materials, apatite compositions, germicides, fetal cells, adult cells, stem cells, enzymes, proteins, hormones, cell therapy substances, gene therapy substances, saline, soap, acid or base solutions, oils, enzymatic solution, 2 or 3 phase mixtures, suspensions, honey, citric acid, baking soda, inert particulate, ceramic particulate, diatomaceous earth, hydrogen peroxide, and/or combinations thereof. Pharmaceutical agents may be configured to generate a chemical or thermal reaction in the body. Therapeutic substances may be combined with the materials used to make the embodiments herein or provided through a reservoir, cavity, passage, or pores of the embodiments herein. Alternatively, the therapeutic substances may be impregnated or coated on the device. Time-released and/or acoustically-released therapeutic substances and drugs may also be incorporated into or coated on the surface of the device. The therapeutic substances may also be placed in a bioabsorbable, degradable, frangible, or biodegradable material, for example a polymer. Therapeutic substances may be placed in one or more layers for incremental and/or controlled release. Therapeutic substances may be delivered orally, percutaneously or transdermally. Therapeutic substances may be positioned with a guidance device, for example a bronchoscope, arthroscope, ureteroscope, cystoscope, endoscope, fluoroscopy, and/or combinations thereof (i.e. endoscopic retrograde cholangiopancreatography (ERCP)). Embodiments of the present disclosure may also include any devices and methods disclosed in U.S. Patent Application Publication No. 2007/0141106, titled “Drug Eluting Implant”, which is hereby incorporated by reference in its entirety. Embodiments herein may be configured to control the environment of the treatment area, for example to warm local body tissue. Embodiments may include any devices or methods disclosed in U.S. Patent Application Publication No. 2008/0086072, titled “Methods and Devices for Controlling Biologic Microenvironments” and U.S. patent application Ser. No. 13/559,352, titled “Methods and Systems for Controlling Medical Environments”, which are hereby incorporated by reference in its entirety.

Embodiments may include, be coated with, and/or provide bacteriacides or be configured to provide bacteriacides to a portion of the body. For example, bacteriacides include antibiotics or hydrogen peroxide to enhance the removal and/or prevention of biofilm. Bacteriacides may include any agent that kills bacteria or slows or resists the growth or reproduction of bacteria, for example disinfectants, bacterial antiseptics, bacterial antibiotics, or any combination thereof. Bacteriacides may reduce, kill, or resist formation of pathologic material, for example biofilm. Embodiments may include an injection and/or suction port, for example, to inject bacteriacides and/or remove pathologic material.

An exemplary use may include reduction of pathologic material in a treatment area including a passage or cavity of the body, for example a sinus. For the purposes of the present disclosure, passage and cavity are used interchangeably herein to mean any space, which may be unoccupied or occupied. Embodiments may include an effector configured to access the treatment area, for example an entirely or partially occluded cavity. In an embodiment, the effector may include an elongated portion positionable through a passage of the body to reduce, ablate, and/or disintegrate pathologic material of an occlusion, irrigate fluids and/or therapeutic substances into the treatment site, remove the pathologic material, and/or dilate the body passage. In another embodiment, the effector may include a projector, for example, a barrel staved acoustic projector or ring projector. Embodiments may be configured to penetrate an occlusion, for example a blocked or collapsed cavity. Embodiments may include a flexible effector configured to transmit vibratory and/or acoustic energy to disintegrate pathologic material in the occlusion. In addition, embodiments may include an irrigation system configured to remove and/or (i.e. flush) pathologic material from or provide (i.e. inject) therapeutic substances to the treatment area.

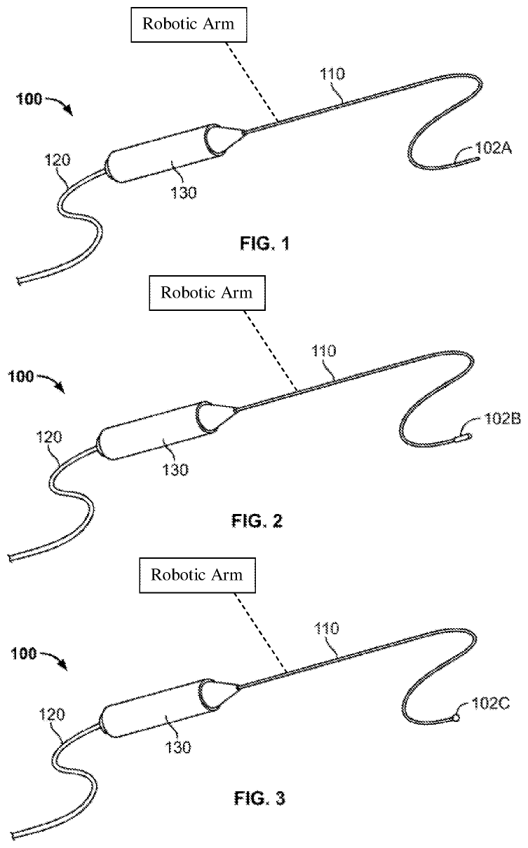

Embodiments may include a handpiece 100. (FIG. 1). Handpiece 100 may include effector 110, energy source 120, and/or handle 130. Effector 110 may include distal portion 102. Distal portion 102 may include any configuration, shape, or material disclosed herein or known in the art. Distal portion 102 may include a similar shaped cross section relative to the remainder of effector 110 (FIG. 2) or a different cross section relative to the remainder of effector 110 (FIG. 2-3). In an embodiment, distal portion 102B may include a continuous, rounded, circular, and/or polygonal (not shown) cross-section and/or a cylindrical or polygonal shape. (FIG. 2). In another embodiment, distal portion 102B may include a continuous, round, circular, and/or polygonal (not shown) cross-section and/or a cylindrical or polygonal (not shown) shape. (FIG. 2). In a further embodiment, distal portion 102C may include a varying or asymmetrical (not shown) cross-section and/or a spherical or obloid shape. (FIG. 3).

Energy, for example vibratory energy, may be applied to, through, and/or irradiate from effector 110. Effector 110 may vibrate with respect to tissue and/or urge distal portion 102 against and/or through tissue. Effector 110 may be configured to irradiate energy from and/or affect tissue in contact with and/or a distance from effector 110. Effector 110 may be configured to apply energy to tissue, for example, to modify, ablate, create, and/or widen a passage or cavity. Furthermore, effector 110 may be configured to remove pathologic material from a portion of the body, for example a cavity or passage. Embodiments may be applied to body tissue of any portion of the body including any soft tissue or hard tissue (i.e. bone).

Effector 110 may emit harmonics that are multiples of a drive frequency from energy source 120, for example multiples of 1, 2, 3, 4, 5, 6, 7, 8, 9, 10, or more. Effector 110 may emit pressure (i.e. a sound pressure level) at any level, for example from 0-220 decibels. Pressure may emanate any distance from effector 110, for example about or between 1, 10, or 100 centimeters. Effector 110 may emit a pressure having a beam pattern configured to produce vibratory energy including acoustic energy. Effector 110 may emit pressure sufficient to disrupt, remove, or kill pathologic material (i.e. biofilm). Effector 110 may be configured as an erosion tool, streaming tool, and/or high intensity sound projector. Any or all of the length of effector 110 may be flexible and/or excitable.

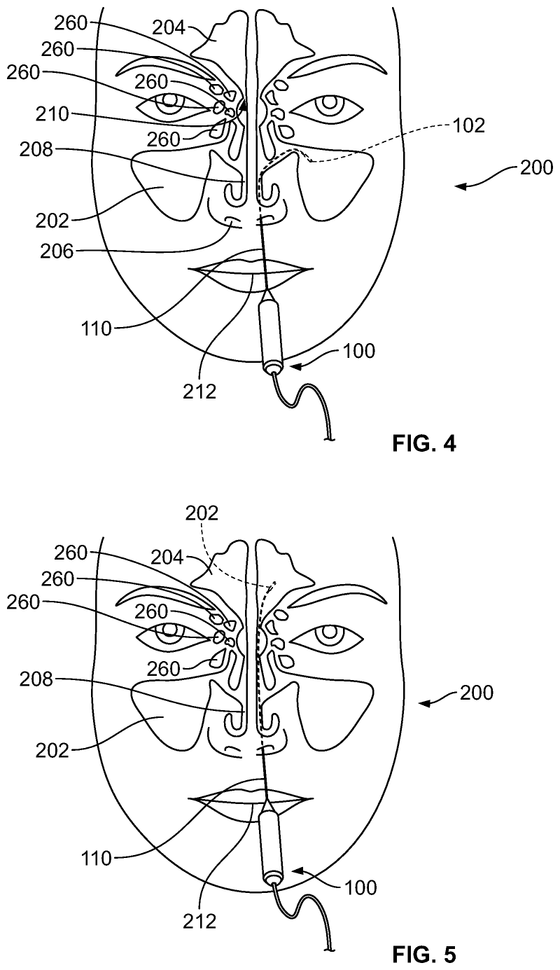

Effector 110 may be positioned in or transmit energy to any portion of the body, for example sinus cavities. (FIGS. 4-7 and 11-12). Effector 110 may be configured to percutaneously or transcutaneously transmit energy. All or any portion of the embodiments, for example effector 110, may be flexible, bendable, curvable, curved, pointed, and/or otherwise configured to access any portion of the body. Effector 110 may also transmit energy through body tissue to access remote cavities of the body. Effector 110 may be configured to target pathologic materials and/or to preserve healthy body tissue. Effector 110 may include attachments and/or accessories configured for diagnostic, imaging, or therapeutic ultrasound. For example, one or more effector 110 may be flexible, malleable, or allow changes in angles to improve treatments, imaging resolution, and ergonomics.

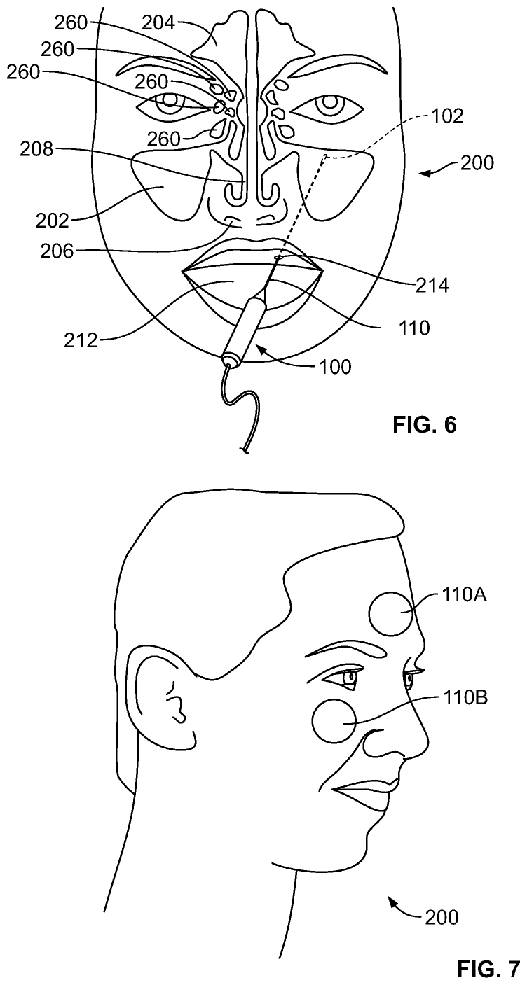

Embodiments may be performed with minimally invasive techniques. For a frontal sinus, an incision may not be required. For a maxillary sinus, a hole, for example 0.25 to 2.0 mm, may be created in palate 214 (FIG. 6) or above and between the roots of teeth (not shown) of mouth 212. Embodiments may be performed with or without applying pressure on the occluded area, pathologic material, body tissue, and/or passage. Effector 110 may be positioned in or through any portion of the body. To access a sinus cavity, effector 110 may be positioned through a natural passage, for example nostril 206 (FIG. 4), or a created passage, for example a hole in palate 214 of mouth 212 (FIG. 6). As an example, effector 110 may be positioned in and/or through nostril 206, nasal passage 208, and/or maxillary sinus 202. (FIG. 4). In another example, distal portion 102 may be positionable into and/or through nostril 206, nasal passage 208, tissue area 210, and/or sinus 204, for example to access frontal sinus 204. (FIG. 5). As a further example, distal portion 102 may be positionable in and/or through mouth 212, palate 214, and/or maxillary sinus 202. (FIG. 6). In an additional example, distal portion 102 may be positioned through nostril 206 or palate 214, through tissue surrounding sphenoid sinus 280 (FIG. 12) or ethmoid sinus 260 (FIGS. 4-6 and 11), and into the sphenoid sinus 280 or ethmoid sinus 260.

One or more emitter 110 may be configured to transcutaneously focus and/or transmit energy. (FIG. 7). Emitter 110 may be configured to transmit energy, for example ultrasonic energy, to a portion of the body. Energy may be configured to focus energy on of a body portion or pathologic material and/or may be configured to preserve healthy tissue or desirable materials. Emitter 110 may be externally positioned (FIG. 7) or internally implanted in any portion of the body. Emitter 110 may include a high-intensity focused ultrasound transducer (“HIFU transducer”). Emitter 110A may be configured to be positioned on a forehead or another external body portion and/or transmit energy to a sinus cavity or another internal body portion underlying the forehead, for example frontal sinus 204. Emitter 110B may be positioned on the cheek and/or configured to transmit energy to a sinus cavity underlying a cheek, for example maxillary sinus 202. Emitter 110A and/or 110B may also be configured to transmit energy to sphenoid sinus 280 (FIG. 12) and/or ethmoid sinus (FIGS. 4-6 and 11). Two or more emitters 110 may simultaneously, cooperatively, and/or sequentially to apply energy.

Effector 110, for example including a HIFU transducer, may be configured to ablate tissue and/or pathologic material a distance from effector 110. Effector 110 may transcutaneously apply energy to pathologic material, for example mucus or tumors. Effector 110 may be positioned exterior to the body and transmit energy into the body, for example to reduce pathologic material that is remote from effector 110. Effector 110 may be configured for lithotripsy to reduce and/or break up pathologic material in the body, for example hardened masses. Embodiments may be configured to reduce hardened masses of any portion of the body, for example a kidney (i.e. kidney stones), gastrointestinal system (i.e. bezoars), and/or biliary tract (i.e. gallstones). As another example, effector 110 may be positioned on a portion of the body, for example on skin, and apply energy to reduce scar tissue. Effector 110 may focus energy toward the body portion to reduce or break up pathologic material. Embodiments may include any devices or methods disclosed in U.S. Patent Application Publication No. 2006/0064082, titled “Minimally Invasive Therapeutic System”, which is hereby incorporated by reference in its entirety.

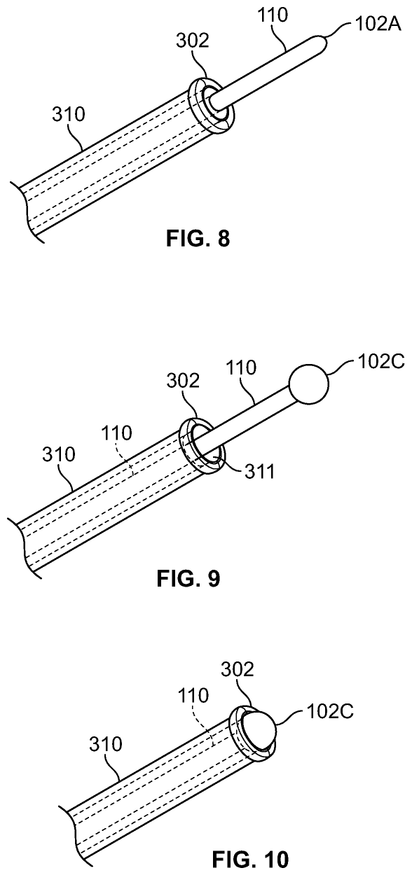

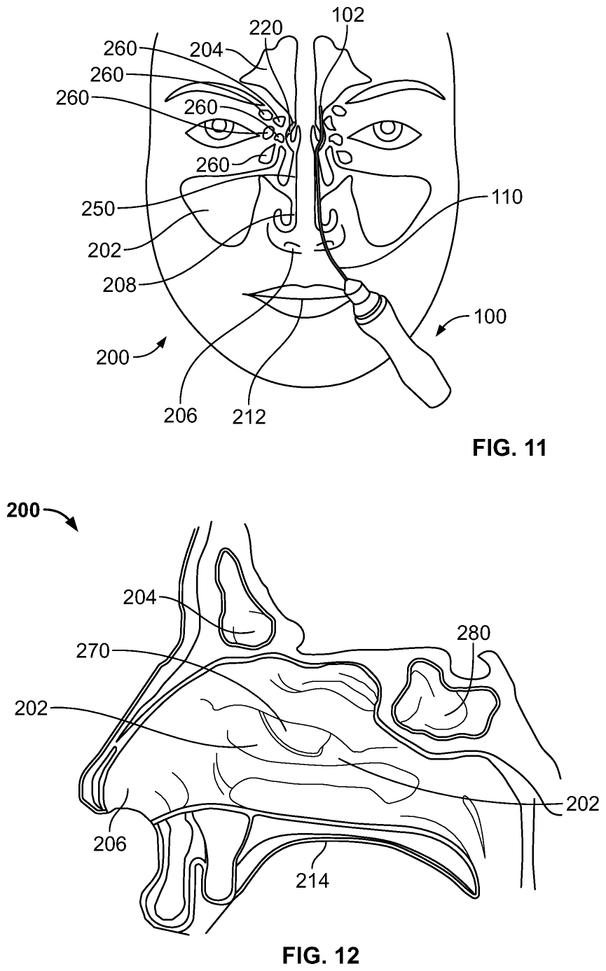

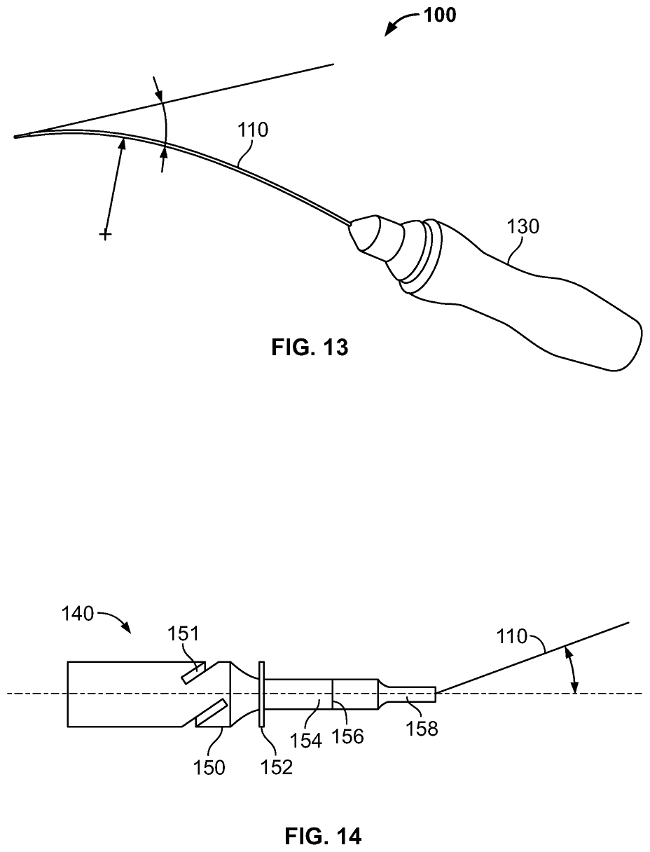

Embodiments may include and/or be used in conjunction with cannula 310. (FIG. 8-10). Cannula 310 may include a leading portion 302, which may include a transducer. Leading portion 302 may include and/or be configured for intravascular ultrasound (IVUS) and/or diagnostic ultrasound. Cannula 310 may include cannula passage 311 and/or be configured to receive effector 110. Cannula 310 may be configured to shield effector 110, for example, to resist application of energy to and/or contact with healthy body tissue along a passage. Effector 110 may utilize leading end 102A to penetrate body tissue. Effector 110 may have a retracted configuration (FIG. 10) for positioning and an extended configuration for energy application (FIG. 9). Cannula passage 311 may include a seat, for example, to receive and/or contour to distal portion 102 during the refracted configuration.

Embodiments may be configured to access and/or penetrate pathologic material in occluded area 220. (FIGS. 11-12). Occluded area 220 may be located anywhere in the body. For example, occluded area 220 may be located in or near nasal passage 208, maxillary sinus 202, passage 220, passage 250, and/or frontal sinus 204. (FIG. 11) Embodiments may be configured to apply energy to a targeted pathologic material (i.e. biofilm) while preserving healthy tissue (i.e. cilia). Effector 110 may include an elongated portion having a wire shape. Handpiece 100 is configured to be positionable in or near occluded area 220 and/or may include transducer 140 configured to respond to energy (i.e. vibratory energy) from energy source 120. Energy source 120 may include an electric generator or a battery in handle 130. Upon application of energy, the effector 110 is excited thereby vibrating effector 110 relative to the occluded area. Pathologic material in occluded area 220 may be eroded by vibration and/or optionally removed (i.e. flushed) by fluid irrigation. As a result, occlusions may be penetrated and/or removed thereby promoting integrity and health of the treatment area.

Effector 110 may include an elongated portion that is dimensioned access to the treatment area. Effector 110 may have any diameter. For example, an approximate diameter may be at or between any of 0.05, 0.6, 0.7, 1.6, 1.7 (5 French), 2, or more millimeters. Embodiments may have any length. For example, an approximate length may be at or between any of 1, 100, 175, 200, or more millimeters. The dimensions of effector 110 may be configured to utilize acoustic streaming, wherein momentum from effector 110 and transducer 140 is transferred to the fluid surrounding effector 110, for example, to enhance flow of irrigation fluid, fluid erosion, improve introduced kinetics of therapeutic substances, and evacuation of the treatment area.

In operation, energy, for example vibratory energy, may be applied to effector 110 to cause vibratory and/or acoustic interaction between effector 110 and the pathologic material. Vibratory energy may include low power density vibration energy resulting from mechanical energy that is converted to thermal and acoustic energy. Low power density vibration energy may be, for example, as low as 0.1 Watt. Application of vibratory energy may result from the conversion of mechanical energy to thermal and/or acoustic energy. The interaction may cause the transfer of vibration, friction, and/or irradiated energy to pathologic material. Energy may be applied to move effector 110 to erode pathologic material of an occlusion, dilate a passage and/or stimulate a dilation response in the passage. Embodiments may be configured to substantially preserve healthy tissue underlying the pathologic material.

Handpiece 100 may include transducer 140, one or more transformer(s) 150, slot 151, boundary 152, attachment 156, and/or booster 158. (FIG. 14). Energy may be generated with transducer 140. Transducer 140 may include crystals, for example, including piezoelectric, ferroelectric peizoceramic materials or any other materials disclosed herein or known in the art. Crystals may include lead zirconate titanate (PZT), and/or comparable materials. Transducer 140 may be configured into a Langevin type. Crystals may be pre-stressed into compression to resist separation during oscillation. Crystals may be poled, electrically configured in parallel, and/or configured to be excitable in the axial direction or any other direction. In another embodiment, handpiece 100 may include a magnetostrictive drive (not shown), a ferromagnetic and/or magnetostrictive material (i.e.Terfenol-D), and/or may be excited by an inductive magnetic field.

Embodiments may also be configured to operate at any energy characteristics, for example any power, voltage, and/or frequency. Embodiments may utilize a power from about 0.5 to 100 W, preferably about 10 watts, a voltage from about 1 to 1000 volts, preferably about 5-50 volts, and an operating frequency from about 20 Hz (sonic) to 200 kHz (ultrasonic), preferably about 70 kHz. Embodiments may also utilize energy characteristics outside these ranges, for example a frequency of beyond 200 kHz, into the megahertz range, and/or into the gigahertz range. Embodiments may include class A, B, AB, C, D, E or other amplifiers or any other amplifier known in the art. Embodiments may also be configured to be disposable, sterilizable, and/or autoclavable. Embodiments, for example transducer 140, may be configured for resonance in any number of frequencies, modes, or combinations thereof and/or may be controlled with a feedback control system.

Transducer 140 may be positioned adjacent to transformer(s) 150. (FIG. 14). Transformer 150 may be configured to apportion strain in one or more modes, for example compression, torsion, and/or bending. The strain may be delivered axially from transducer 140. Embodiments may include slits or holes, which may be cut and/or configured to regulate apportioning to each mode. Embodiments may include cuts, beads (not shown), and/or material characteristics configured to induce a one or more modes.

The handpiece 100 may include boundary 152. (FIG. 14). Boundary 152 provides a restraint for physical mounting. Boundary 152 may include a flange and/or increase in diameter. Boundary 152 may be located at a position of minimum strain and/or minimum transverse contraction and/or extension resulting from Poisson effect. Boundary 152 may be configured to reduce the intensity of the Poisson effect and minimize parasitic excitation, heat noise, and wear. Boundary 152 also enables clamping the faces normal to the longitudinal axis of handpiece 100, which may constrain handpiece 100. Boundary 152 may also include a feature to resist rotation about the longitudinal axis of handpiece 100.

The transformer 150 may include booster 154 and/or booster 158. (FIGS. 14-18). Boosters 154 and 158 may include a reduction in the area of the transformer 150 to guide to momentum from the crystals. The reduction in cross-sectional area at a node may substantially retain the momentum and/or increase instantaneous strain in any or all of the nodes along transformer 150. In addition to or as an alternative to cross-sectional area changes, embodiments may include changes in material characteristics (i.e. density and/or modulus of elasticity) along the longitudinal and/or transverse axes.

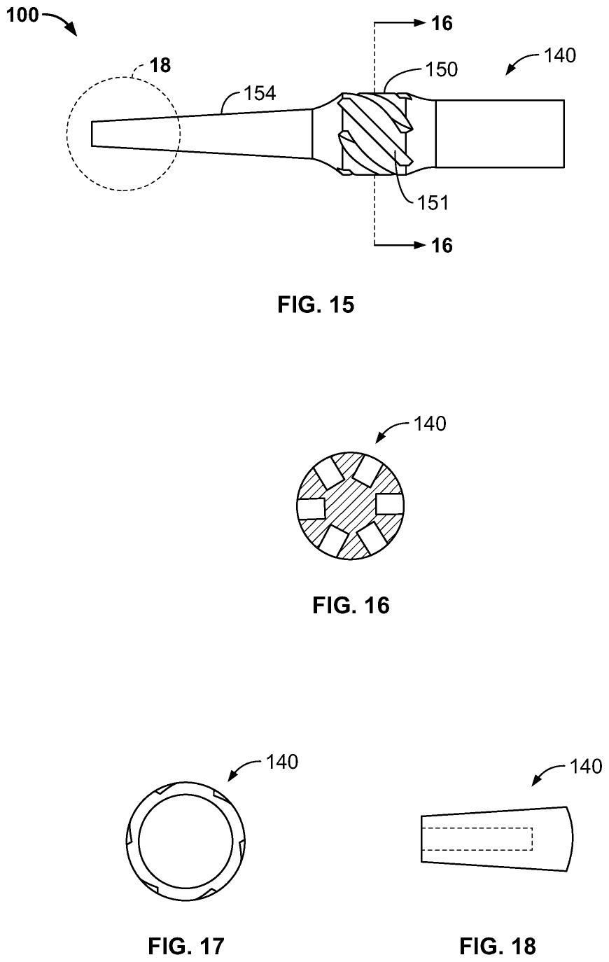

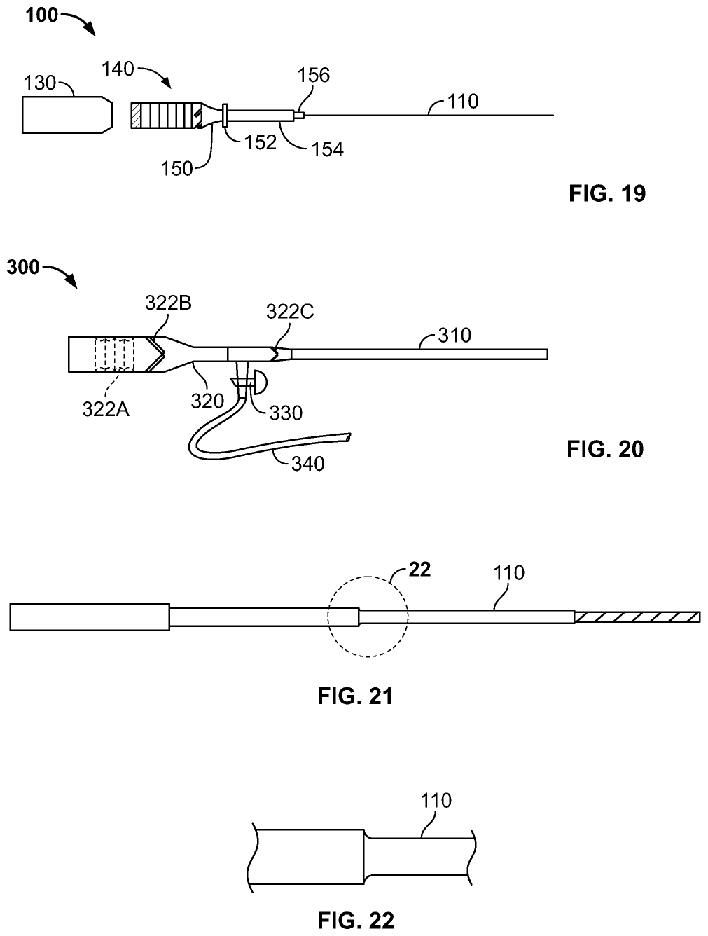

Booster 154 and/or booster 158 may be any length. (FIG. 14-15). In an embodiment, booster 154 may end at distance of one-fourth of a wavelength of the normal operating frequency from boundary 152, although there may not be a visual reference for this location. (FIG. 14). Booster 158 may be connected to the end of booster 154 and/or extend for any length, for example an additional one-half wavelength. Booster 158 may be connected to transformer 150 with attachment 156, which may include a press fit, threads, adhesive, clamp, weld, collet type, or any other connection disclosed herein or known in the art. Attachment 156 may connect transformer 150 and booster 158 with a relatively low force. Attachment 156 may be configured to accommodate acceleration of the connecting surface and/or resist relative motion between transformer 150 and booster 158. This transformer may further apportion various modes along its length. Transformer 150 and/or booster 158 may include two or more different materials, for example a composite of titanium and tantalum. Transformer 150, booster 154, and/or booster 158 may include a shape configured to accomplish transformation, for example, of a velocity vector of a wave guide particle along the vibrating system and/or of an effector with respect to with a contact surface of a tissue.

Effector 110 may be attached to booster 158. (FIG. 14). Effector 110 may be mounted off center and/or at one or two angles with respect to the longitudinal axis of handpiece 100, which may increase torsional and/or transverse vibration in response to axial strain. All or any portion of embodiments (i.e. the transducer, transformer, booster, crystals, and/or effectors) may have any round, polygonal, circular (shown), hexagonal, square, rectangular, symmetric, and/or asymmetric cross-sectional shape.

Embodiments may include cannula assembly 300. (FIG. 20) Cannula assembly 300 may include cannula 310, port 320, seal 322, valve 330, and/or fluid line 340. Cannula assembly 300 may be configured to receive handpiece 100. Seal 322 may intrude any number of inwardly protruding rings (i.e. seal 322A) and/or flexible flaps (i.e. seal 322B and 322C) to allow effector 110 to be positioned through cannula assembly 300 while substantially maintaining a fluid seal against effector 110. Fluid line 340 may be configured for suction and/or irrigation, for example, of saline, therapeutic substances, or another fluid. Cannula assembly 300 may serve as a fluid boundary for effector 110 and/or an attachment location for irrigation of fluids. Cannula assembly 300 may provide a housing for a physical oscillator (not shown) to move seal 320, which may reduce absorption of transverse vibration by seal 320. Cannula 310 may be configured to restrain the vibration of effector 110 and/or selectively expose the distal portion of effector 110. Cannula 310 may be flexible and/or be configured in a fixed curvature suitable for a specific anatomical structure. Cannula 310 may contain a positioning system to articulate the distal portion of cannula 310, for example a magnetic or robotic system. Cannula 310 may include dimensions and/or material characteristics that are configured to enhance the desired modes of vibration. The length of cannula 310 may be dimensioned and configured to limit the amount of effector 110 that is exposed to the treatment site. Cannula 310 may be configured as a boundary for the irrigation fluid, for example, by allowing fluid flow through the walls of cannula 310, along the axis of cannula 310, and/or remain trapped within the cannula 310. The fluid may be any liquid, gas, or any combination thereof.

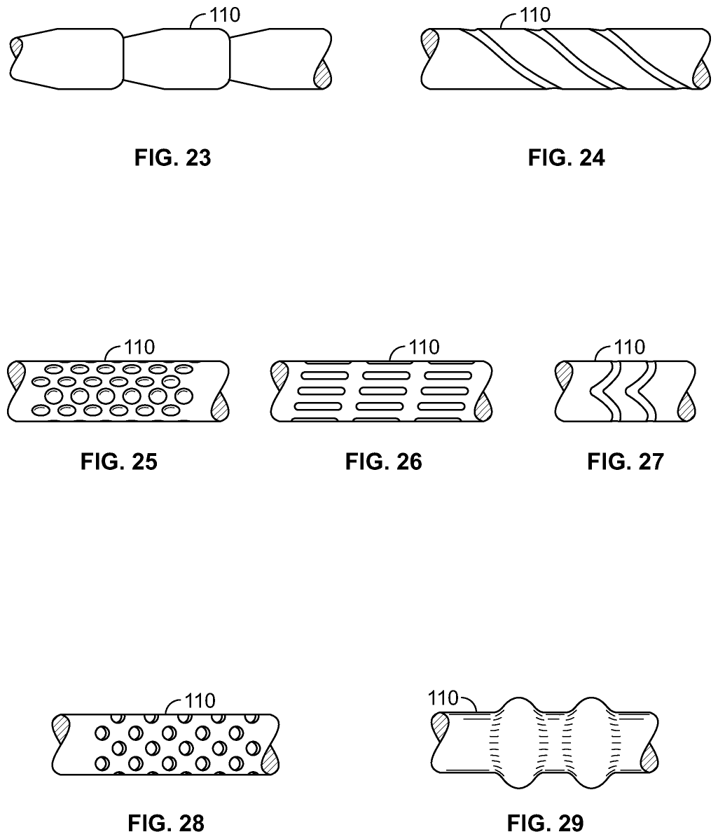

Effector 110 may include one or more types of surface features, for example steps (FIG. 21-22), abrasive features, (FIG. 21), nested cones (FIG. 23), helixes (FIG. 24), dimples (FIG. 25), flutes (FIG. 26), chevrons (FIG. 27), holes (FIG. 28), and/or bumps (FIG. 29). Embodiments may include a surface pattern, for example on effector 110 and/or cannula 310. The pattern may be embedded in and/or protrude from surface. The pattern may be masked stenciled, chemical etched, forged, hammered, or cast. The pattern may include a helix, grid, grooves, bumps, or another surface pattern. The pattern may be on the inner or outer surface.

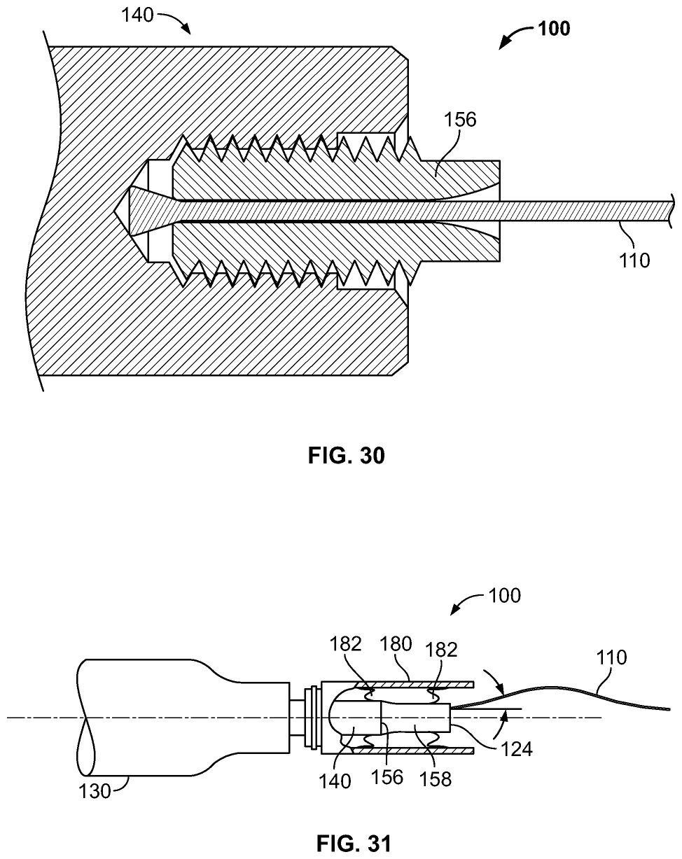

Effector 110 may be the principle energy transmission device that carries vibratory energy including acoustic waves from the transducer 140 to the treatment site. Effector 110 may be attached to the transducer 140 and/or transformer 150 with the attachment 156, for example, including a mechanical interference, taper-fit, male taper to female taper fit, ball-fit, press-fit, welded, adhesive, and/or any connection disclosed herein or known in the art. (FIG. 30). Attachment may include a strain relief surface to allow the effector 110 to bend as it exits attachment 156. In use, effector 110 receives momentum from transducer 140 and translates energy through effector 110. Energy may be contained and/or controlled by an inner surface of cannula 180/310 and/or dampers or seals 182 of cannula 180, which may be integrated into and/or removable from handpiece 100. (FIG. 31) Energy and/or momentum may be absorbed by cannula 180/310, the irrigation fluid, and/or dissipated in effector 110, but a majority or at least a substantial portion may be transferred to the treatment site.

Effector 110 may be any material disclosed herein or any material suitable to facilitate energy and/or momentum transfer. For example, effector 110 may include an elastic material. Effector 110 may also include titanium, titanium alloys, and/or high modulus materials. As other examples, effector 110 may include aluminum, nitinol, steel, brass, carbon fibers, glass, high modulus plastics, composites, liquid metals like mercury, and/or water. Effector 110 may include non-toxic and/or biocompatible materials. Effector 110 may include materials having a higher modulus of elasticity with an acoustic wave guide having a lower tangent or lost modulus. Polymers may have a higher loss modulus, may heat during stress cycling, and/or may be suitable for short durations or to deliberately limit the duration of use.

Effector 110 may be flexible, for example, to be able to navigate the passages of the body. As effector 110 is reduced in diameter, the stress in effector 110 may be reduced for a given bend radius. The smaller the diameter of effector 110, the less stress it may endure from bending. A smaller effector 110 may translate less energy, for example, due to reduced mass. Effector 110 may be configured for appropriate flexibility and/or for the thickest diameter suitable for use.

Effector 110 may also have a cross-section of any shape, for example round, elliptical, triangular, square, any polygonal or round shape, and/or any other shape disclosed herein or known in the art. Effector 110 may have a cross-section that changes along its length, for example, in the active and inactive sections of the effector. All or any portion of effector 110 may have a mass is offset from the transverse and/or longitudinal axes, for example, to promote desired motion or configuration. This may result in mutually orthogonal area moments of inertia (AMI) that are unequal. Effector 110 may be configured with a mass and/or area moment of inertia imbalance with respect to multiple transverse and/or longitudinal axes of the effector 110, which may force effector 110 to actuate and/or bend to a desired configuration. The imbalance may result in two or more discrete natural frequencies near each other. Effector 110 may be processed (i.e. ground) with the mass and/or AMI varying along the length. Effector 110 may be weighted and/or clad in a coating to alter its vibrational characteristics. A section thickness increase and/or the mechanical attachment of a weight may force a location on effector 110 to vibrate less than another section of effector 110. Effector 110 may have an active section in the plane normal to the longitudinal axis. This may create a chorus of natural tones, which may be excited and/or act therapeutically. In combination with a control system, an adaptive vibrational system can resonate in any range of frequencies. Effector 110 may also be configured to act as a mechanical booster and/or transformer.

Effector 110 may be configured to enhance flow of fluid along its longitudinal axis, which may be referred to as streaming. Streaming may result from the difference between the relative incompressibility and lower vapor pressure of the fluid, which may transfer momentum from effector 110 to the fluid. Flow may also be induced due to what is known as Stokes oscillating boundary layer. On relatively smooth sections of the effector, flow may be biased toward the lower net velocity sections of effector 110. Since there are several higher and lower velocity sections on the effector 110, a multitude of fluid movement strategies can be created by the placement of check valves, flow restrictors, vents, bypasses and seals. Flow can be diverted away from a lower velocity point of effector 110 and reintroduced at a higher velocity point of effector 110 through a bypass. This can be repeated along the length of the effector 110 in multiple stages so that all or any desired portions of effector 110 streams fluid.

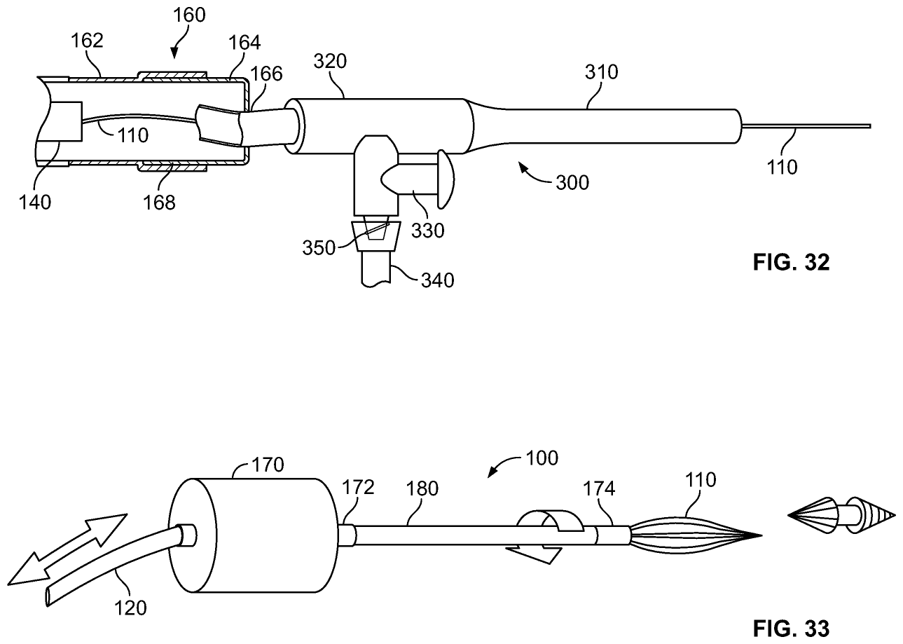

Cannula 310 may assist in delivery of energy to the treatment site. (FIG. 32). Effector 110 may be contained by cannula assembly 300. Cannula assembly 300 may assist effector 110 in withstanding external environmental forces and internal strains. In multimodal system, the total strain of a section of effector 110 may be relatively large, especially considering the size and diameter of effector 110. Also, effector 110 may be exposed to strain at a higher number of cycles. Cannula assembly 300 may serve as a boundary for the movement of and strain on the effector 110.

Buckling in effector 110 may be controlled by the diameter of cannula 310 relative to effector 110. For example, if the inner diameter of cannula 310 is configured to match the outer diameter of the effector 110, effector 110 may compress only in an axial direction. If the inner diameter of cannula 310 is increased relative to the diameter of effector 110, effector 110 may buckle in one or more of static modes. For example, when effector 110 is compressed axially within cannula 310, the buckle may include a helical or similar shape from effector 110 with a smaller diameter effector following the inner surface of cannula 310.

In an embodiment, a static mode includes a helix structure. Effector 110 may collapse under axial compression and form a helical structure, wherein the effector is tangent to the inner surface of cannula 310 while spiraling along the length of cannula 310. This mode may enhance ablation of pathologic material. The inner diameter of cannula 310 may resist development of the swirling helix and/or allow effector 110 to develop within the anatomical structure of the body after the effector 110 leaves cannula 310.

The coefficient of friction, size, and shape of the inner surface of all or any portion of cannula 310 may be configured to allow, resist, and/or control motion of effector 110 including the extent of motion and its proximity to pathologic material and underlying healthy tissue. Cannula 310 may control energy transmission of effector 110 with the coefficient of kinetic friction between effector 110 and the wall of cannula 310. The coefficient of friction of the outer surface of cannula 310 may be configured to enable the insertion and/or retention of the effector 110 and cannula 310 with respect to a portion of the body. The cross-sectional shape of cannula 310 may be configured to control energy propagation through effector 110. Cannula 310 may be manufactured by being rolled and/or twisted onto a spool and allowed to cool, which may result in a section that is out of round and/or oval. When a cannula 310 of this shape is used, it may filter the motion of effector 110 to a transverse motion in the direction of the longitudinal axis of the section of cannula 310.

Cannula 310 may include any material disclosed herein or any other material acceptable for medical use. Embodiments may include materials with a densitiy, stiffness, flexibility, and/or durability configured for delivery of fluid and/or energy containment. Embodiments may include stainless steel, Teflon, silicone, glass, urethane, PEEK, other plastics, plastic blends, mixtures, and/or any other material disclosed herein or known in the art. Embodiments may include composite, laminated, coextruded, or layered materials. Cannula 310 may be configured with a material that absorbs or reflects the energy of effector 110. If effector 110 is vibrating back and forth within cannula 310 with limited motion or no swirling, the momentum may be transferred to cannula 310, which may be in accordance to the amount of impedance match between the materials and stiffness of effector 110 and cannula 310. A stiffer and/or harder tube may offer less loss, whereas a flexible and/or harder tube may offer more loss.

Cannula 310 may be configured to support column loading and or articulation to enable insertion of effector 110 into cannula 310 and insertion of cannula 310 into a portion of the body. Alternatively, an excited effector 110 may simulate a static effector of greater diameter and is less susceptible to buckling. This may be accomplished by using cannula 310 of a relatively large diameter and thickness and/or having a higher yield stress, higher modulus of elasticity, and lower creep. Cannula 310 may be configured for the access diameter of the portion of the body, to receive the diameter of effector 110, and for flexibility to navigate through curvilinear pathways of the body. Cannula 310 may include a biodegradable portion and/or a material that selectively degrades once introduced into the body thereby revealing an active portion of effector 110, which may be initiated by contact with a material, state in the body, irrigation fluid, and/or acoustic energy.

Cannula 310 that may include a solid, uniform, continuous, and/or cast extrusion configured to contain effector 110. Cannula 310 may be a composite of co-extruded materials continuously or intermittently placed in sections. Cannula 310 may have one or more side ports to irrigate a portion of the body and/or regulate fluid pressure and/or fluid flow. Cannula 310 may include a screen section that grates pathologic material, allows pathologic material to pass through it, and/or is configured for effector 110 to cut pathologic material into particles. This screen section may be configured to control the mass removal rate for pathologic material, for example mucus, biofilm, and/or tissue.

Cannula assembly 300 may include fluid line 340. Fluid line 340 may be configured to cool effector 110 and the fluid, which may be heated by effector 110. Fluid line 340 may also be configured to dampen the effector, provide lubrication between effector 110 to cannula 310, dilute the pathologic material, remove waste, control the direction of flow, and/or deliver therapeutic substances. Fluid line 340 may include a lock fitting and/or valve 330. Fluid line 340 may be rigidly fixed to or detachable from cannula assembly 300.

Embodiments may include chamber 160 to releasably connect effector 110 and cannula assembly 300. (FIG. 32). Chamber 160 may include effector connection 162, cannula connection 164, cannula opening 166, and interlock 168. Chamber 160 may be sealed relative to effector 110 and/or cannula assembly 310. Effector connection 162 may releasably attach to effector 110, for example with a clamp with a seal. Cannula connection 164 may releasably attach to cannula assembly 100, for example with cannula opening 166 with a seal. Chamber 160 may be expandable in length at interlock 168 and/or include adjustable sealing locations. Interlock 168 may include any attachment features disclosed herein. Chamber 160 may prevent an accidental and/or use-related mechanical grounding of effector 110. Chamber 160 may facilitate reliable operation of effector 110.

Handpiece 100 may include one or more seals. The seal may be configured so that passage of effector 110 through port 320 of cannula assembly 300 may or may not be necessary. Handpiece 100 may be configured so that the pressurized fluid boundary is near a distal portion (“horn”) of transducer 140 and/or effector 110, for example when immersed. The face of the horn may be normal to the fluid. In this embodiment, the fluid in contact the horn may induce stream and/or flow away from the horn and out through the cannula 310. As such, the horn may be utilized as a fluid pump.

Effector 110 may be configured to operate as a blender. (FIG. 33). Embodiments may include energy source 120, motor 170 (i.e. stepper motor), motor 172 (i.e. hollow motor), cannula 180, clutch 174, and effector 110. Effector 110 may be configured as a collapsing wisk shape. Embodiments may be configured to rotate, pulsate, and be stimulated acoustically by transducer 140.

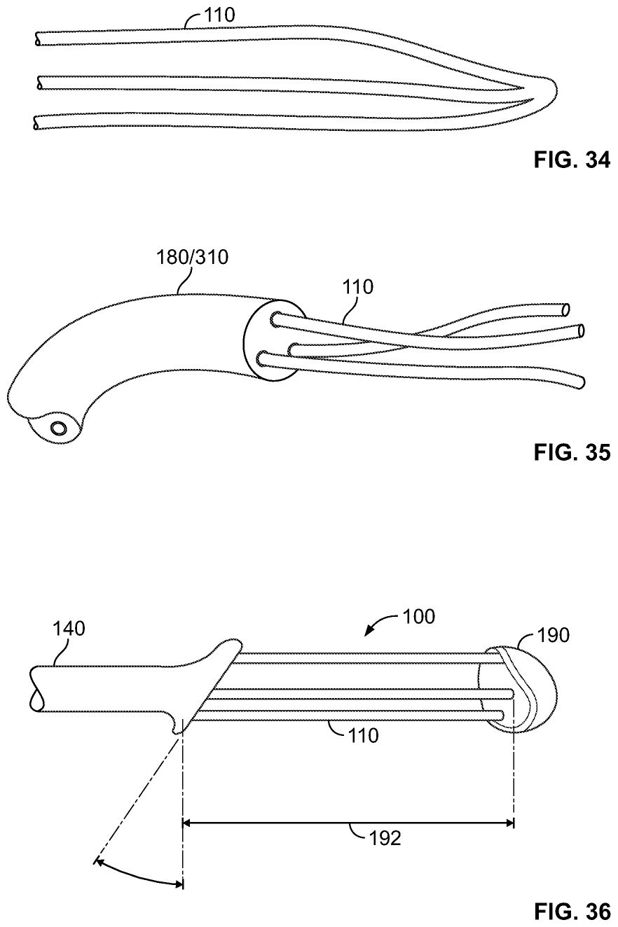

Embodiments may include one or more effector 110 with multiple filaments. (FIG. 34-36). The filaments may combine or branch at any portion along effector 110. (FIG. 34). All or any of the filaments may be configured to excite at any or several different frequencies. Cannula 180/310 may include one or multiple channels to receive multiple filaments. (FIG. 35). The filaments may have a proximal portion or distal portion that is joined (FIG. 34) or free (FIG. 35). One or more effector 110 may be configured to conform to a curved and/or irregular surface.

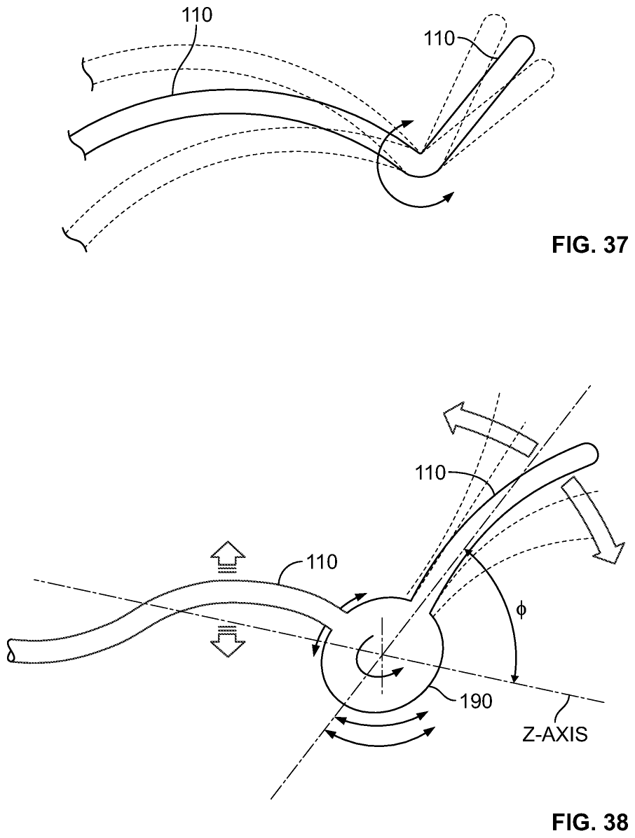

One or more effector 110 may be connected to mass 190, which may oscillate about its center of mass but may not substantially vibrate. (FIGS. 36-38). For certain frequencies, oscillation of mass 190 may reduce, for example, to zero. A kink may be put in effector 110 to create a standing wave on a distal section of effector 110. (FIG. 37). Effector 110 may be attached to the mass 190 and/or vibrated at any angle relative to the z-axis of the effector 110, which may enable effector 110 to vibrate around curves and/or corners. (FIG. 38). Mass 190 may also include a plate or any shape configured to transmit energy to or contact pathologic materials and/or tissue.

Effector 110 and/or cannula 310 may include a coating and/or insert on the inner and/or outer surface. The coating and/or insert may include metal and/or polymer. The coating and/or insert may be configured to increase or decrease the coefficient of friction and/or increase the fatigue life of effector 110 and/or cannula 310. As an example, the effector may include a steel effector having a polymer or ceramic coating.

Embodiments may include a surface wave propagating cannula configured to self-feed into or out of the body. Embodiments may be configured to induce a Raleigh or some other wave or combination of waves in the cannula 310 having a beat frequency to mimic peristalsis. Peristalsis may be generally described as a radially symmetrical contraction and relaxation of muscles, which propagates in a wave down a muscular tube (i.e. a digestive tract of a body). Specifically, embodiments may be configured to propagate a traveling wave on a surface of cannula 310 thereby inducing motion along cannula 310, which may induce fluid flow in cannula 310.

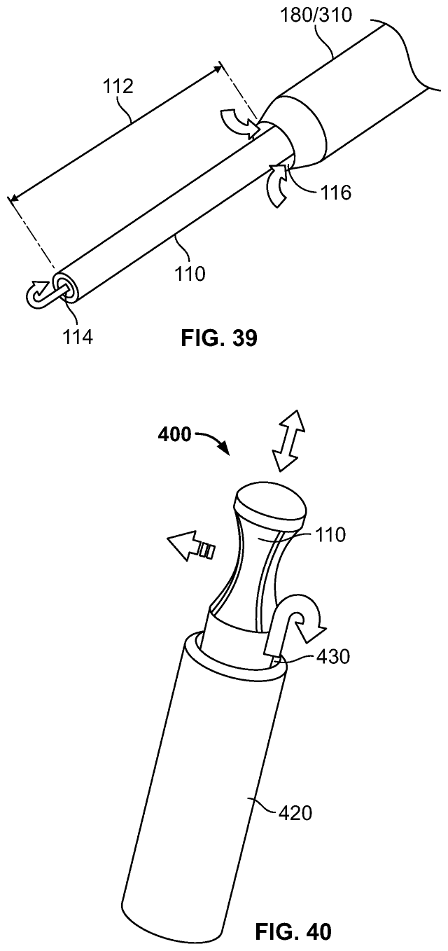

Handpiece 100 may include effector 110, handle 130, channel 114, port 116, cannula 180 of handpiece 100, and/or cannula 310 of cannula assembly 300. (FIG. 39). Handpiece 100 may include active section 112 of effector 110 that extends beyond the cannula 310 and is exposed to pathologic material. Active section 112 may have a length of about 1 mm to 20 cm, preferably 1 to 5 cm, and a diameter of about 0.05 mm to 2 mm, preferably about 1.02 mm (18 gauge) or 1.10 mm (5 French). Effector 110 may include channel 114, which may be configured for streaming, irrigation and/or suction. Handle 130 or cannula 310 may include port 116, which may be configured for streaming, irrigation, suction, and/or therapeutic substances (i.e. pharmaceutical agents). Alternatively, effector 110 may be solid and/or have reversed flow.

With reference to FIG. 40, projector assembly 400 may include effector 110, cannula 420, and/or port 430, which may be configured for suction and/or irrigation. Embodiments may be configured to operate as an acoustic projector system. Effector 110 may include a monopole, dipole, or quadrapole ultrasonic projector. Energy may be applied to effector 110. Effector 110 may be configured to be bendable, flexible, and/or guided through a cannula. In use, projector assembly 400 may be introduced into an occluded area 220 and/or energized thereby vibrating in one or more modes, frequencies, and/or pulses, for example, to degrade pathologic material and/or dilate a passage. Alternatively, projector assembly 400 may be used to radiate acoustic energy, to kill healthy tissue or infectious organisms. It may operate in frequencies as low as audible and into the gigahertz range. It may be used to pulse and/or mechanically shock the surrounding fluid. Sound and/or ultrasound at the level of 80 to 220 dB may be generated. It may be used to resist repopulation of a region with natural tissue or infectious organisms. Effector 110 may be disposed in and/or guided by cannula 420.

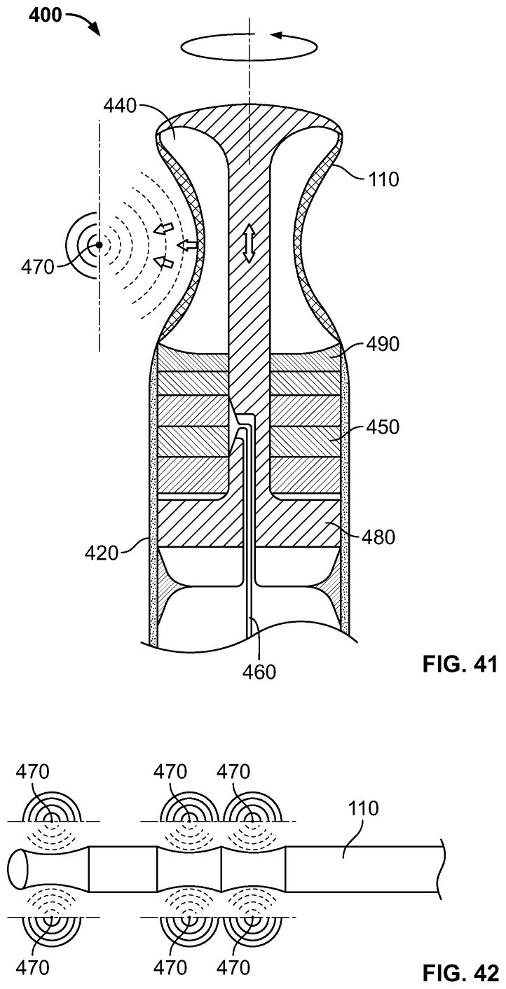

Projector assembly 400 may include effector 110, disposed between a distal portion of spool 480 and transducer assembly 450. (FIGS. 41-42). Effector 110 may be concave (as shown) or convex. Transducer assembly 450 may be connected to effector 110 with spool 480 so that the crystals of transducer assembly 450 may be as large in diameter as the effector 110. Spool 480 may be fitted between the crystal stack of transducer assembly 450 and a proximal end of effector 110. Embodiments may include a prismatic projector, wherein the crystals of transducer assembly 450 would be strips and the spool 480 would be an I-beam. This may enable a beam pattern yields at least two lines of field concentration at point 470. Embodiments may include a plurality of projector surfaces (FIG. 42).

Referring to FIG. 30, effector 110 may be retained by either a press fit or a swage fit with respect to the distal portion (the “horn”) of transducer 140 and/or effector 110. Effector 110 may be swaged on the end and/or captured within the diameter of transducer 140 with a set screw. Tightening of the set screw may pinch the end of effector 110 between the set screw and the horn thereby resisting movement between the screw and the horn. Alternatively, the set screw (not shown) may be a slit tube pressed between effector 110 and the horn. The slit tube applies an inward radial force to effector 110. The slit tube may include any material disclosed herein, for example steel, titanium, plastic, or cement.

In an additional embodiment, a system may be configured to couple to an occluded area (i.e. nasal and/or sinus cavity) from the exterior of the body (i.e. external surface of the nostril, cheekbone, and/or forehead). A resonant system may be created with respect to the occluded area, wherein an air-filled cavity (i.e. occluded area) acts like a spring and contents of the cavity (i.e. air and mucus) act like a mass on the spring. An external speaker or drive system couples with the occluded area and a resonant field is generated thereby oscillating the pathologic material (i.e. mucus, biofilm, and/or pathologic material). A feedback control system may detect, control, and/or maintain resonance of the system.

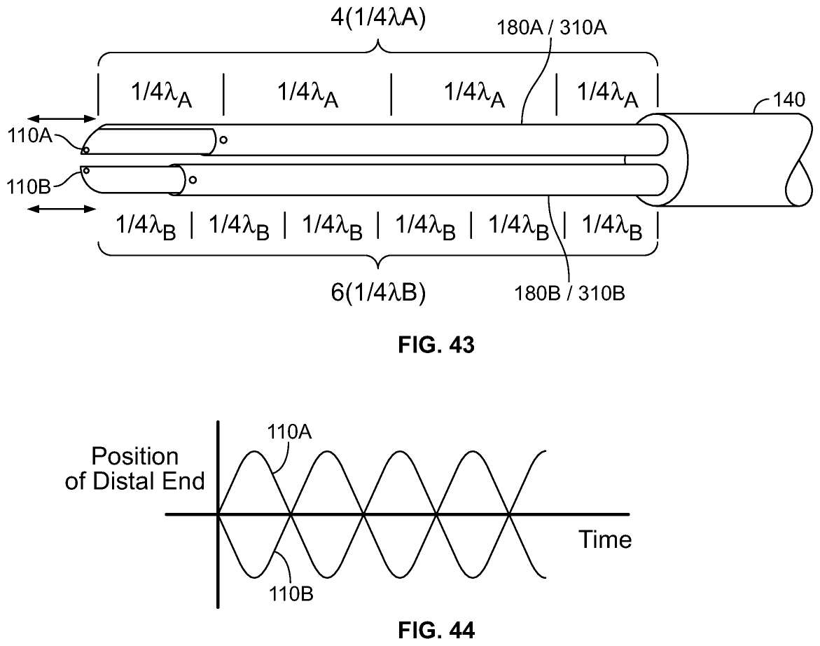

Embodiments may include multiple effectors 110 connected to transducer 140. (FIG. 43) Effector 110A and effector 110B may include different materials, for example any combination of materials disclosed herein, for example titanium, brass, ceramic, glass, contained fluids (i.e. water), mercury, and/or magneto-rhealogical fluids. Embodiments may include cannula 180/310. A plurality of effectors 110 may be connected to a single transducer 140. Alternatively, effectors 110 may be connected to interdigitated or intertwined transducers 150. Effector 110A and 110B may be configured to oscillate at different wavelengths. (FIG. 44). Embodiments may include a phase inverter and/or be configured to give multiples (i.e. twice) of the output at a given frequency.

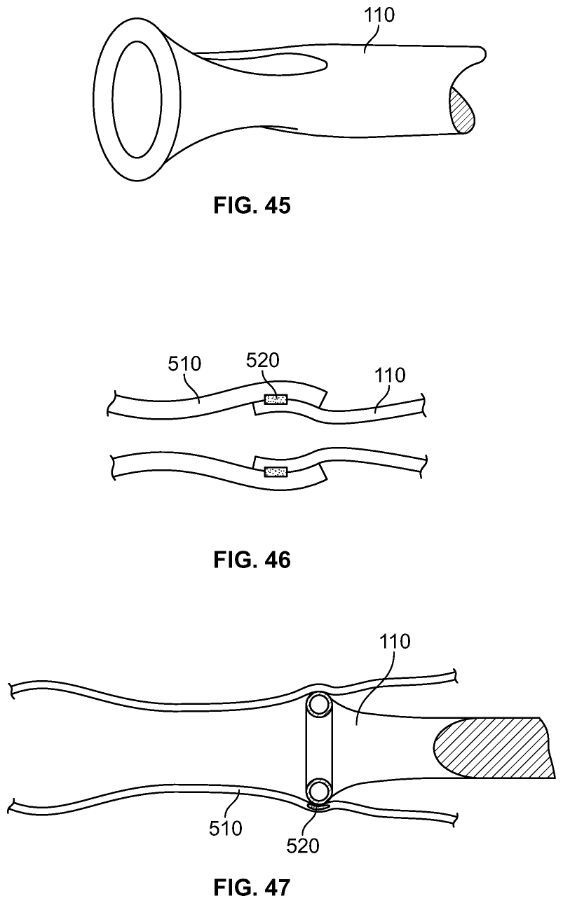

All or any portion of effector 110 may include a torus or funnel shape. (FIG. 45-47). All or any portion of effector 110 may be hollow. Effector 110 may be configured to be positioned in a body passage 510, for example a blood vessel. (FIG. 46-47). Effector 110 may be configured for any medical procedure, a duct or vessel anastomosis. Effector 110 may be used to position an implant, for example a stent. Effector 110 may be used as an implant, for example, to provide a conduit for blood, pathologic material, or other body tissues. Effector 110 may be configured to ablate and/or coagulate tissue 520. Effector 110 may disrupt, ablate, and/or coagulate the membrane and/or stress carrying capability of tissue from the transfer of energy from effector 110 to the tissue. Effector may include a variable and/or adjustable diameter or length, for example, to allow access to a treatment area within the body and/or expansion to a larger diameter to ablate the surface of a larger cavity. (FIGS. 46-47).

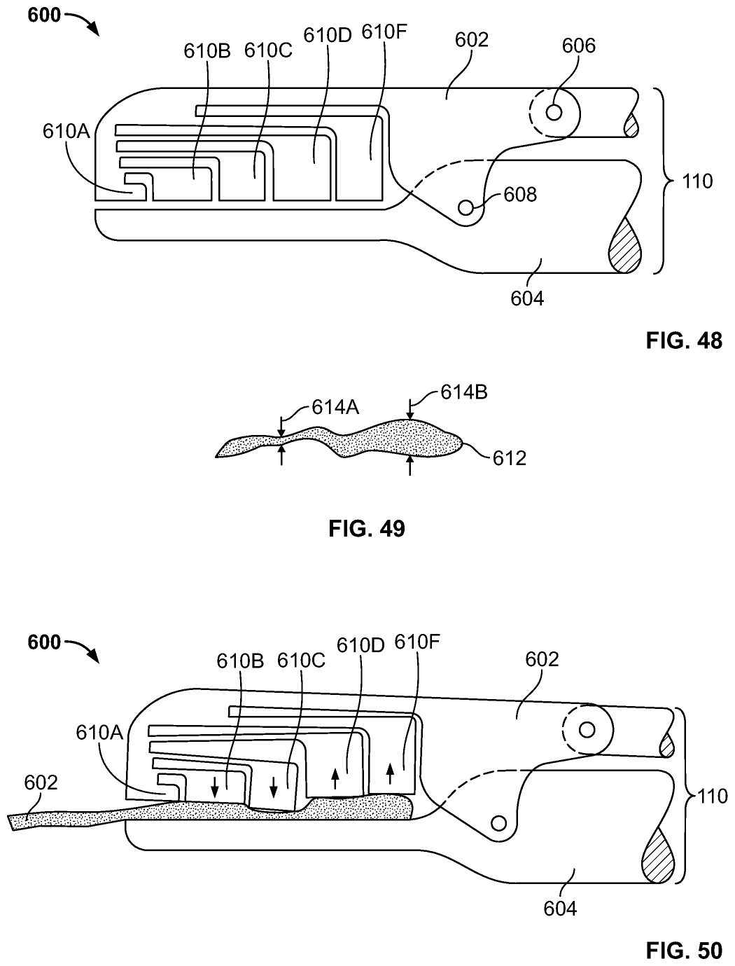

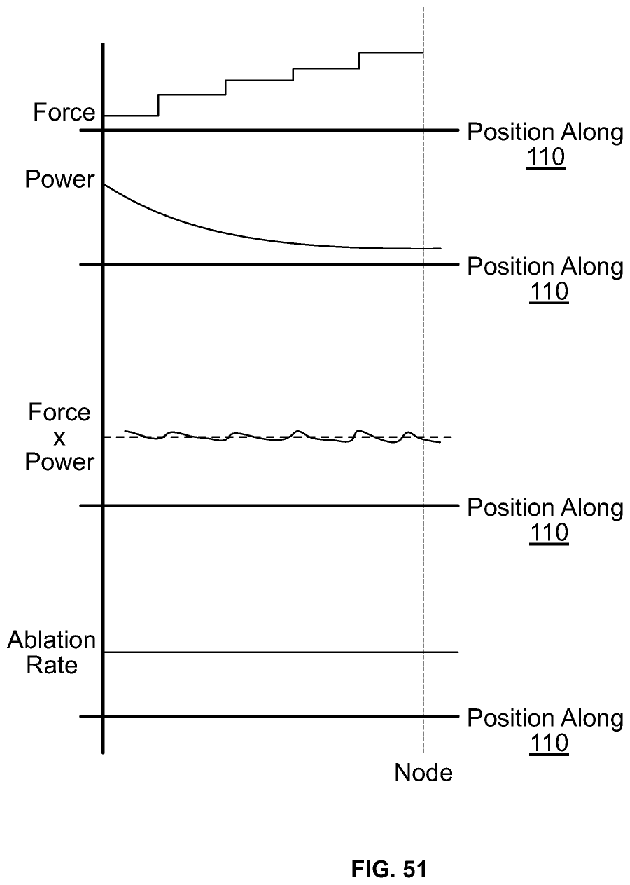

Effector 110 may include a first member 602 and a second member 604. (FIG. 48-50). First member 602 may be connected to a first energy emitter, for example a mechanical actuator. Second member 604 may be connected to a second energy emitter, for example a vibratory energy device. First member 602 and/or second member 604 may include one or more of elements 610, for example elements 610A-610F. Elements 610 may be configured to provide substantially a pre-defined and/or substantially uniform force and/or pressure. Elements 610 may be configured to individually deflect to account for the thickness and/or density of body tissue 612. (FIG. 49). For example, a tissue 612 may include a first tissue thickness 614A and a second tissue thickness 614B. (FIG. 49). First member 602 and/or second member 604 may include elements 610 that are configured for first tissue thickness 614A and/or second tissue thickness 614B. Elements 610A to 610F of first member 602 may be configured to apply a force at each element that may depend on or vary inversely to the energy output at contact points on second member 604. Second member 604, opposing elements 610, may vary in its surface velocity long its length, which may normalize an ablation rate of tissue with respect to the position along second member 604. (FIG. 51)

Effector 110 may be configured to selectively remove, erode, abrade, cut, and/or pierce target types or areas of desirable material (i.e. pathologic material) and/or selectively preserve other types or areas of undesirable material (i.e. healthy tissue). For example, effector 110 may include a surface or hardness determining feature configured to resonate with respect to or against the target tissue and/or pathologic material (i.e. bone, mucus, and/or biofilm) thereby forming opening 706. Effector 110 may also be substantially dampened with respect to the preserved tissue 708 (i.e. soft tissue, skin, or cilia) thereby promoting healthy tissue function. For example, effector 110 may be configured to remove bone and/or preserve soft tissue. Alternatively, effector 110 may be configured to remove soft tissue and preserve bone. Effector 110 may be configured to target and/or preserve any material disclosed herein or known in the art.

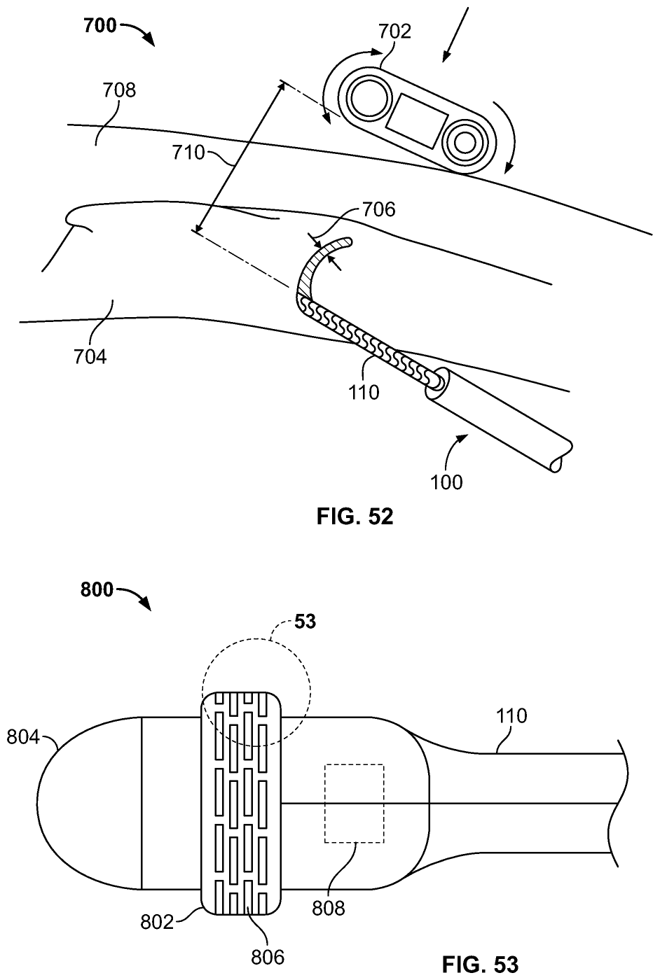

In another embodiment, guidance unit 702 may be configured to guide effector 110 to the treatment area. Guidance unit 702 may include an imaging device, and/or magnetic field generator and/or detector. (FIG. 52). Guidance unit 702 may be configured to detect or position effector 110. Effector 110 may be positioned at distance 710. Guidance unit 702 may be configured to position effector 110 to form opening 706, which may be along a desired, predefined, or intraoperatively adjusted path. Effector 110 may include an emitter and/or detector configured to communicate a signal with guidance unit 702. The signal may include magnetic energy, light (i.e. visible, infra-red, or UV light), sonic or ultrasonic energy, x-ray energy, electric field, or radioactive sources.

Imaging devices may be used to indirectly visualize, guide and/or change the orientation or position of embodiments herein. Examples of imaging devices may include endoscopic guidance, computer assisted navigation, magnetic resonance imaging (MRI), CT scan, ultrasound, fluoroscopic, cystoscopy, endoscopic retrograde cholangiopancreatography cystoscopy (ERCP), X-ray, light detectors, metal detectors, magnetic field detectors, or other visualization device disclosed herein or known in the art. Asymmetric coating, radiopaque markers, or other features identifiable with indirect visualization may be used to identify and/or adjust orientation or position. Indirect visualization may also be used to align portions of a first device relative to a portion of the first device, a second device, or a body portion.

Embodiments may be configured to utilize any type of energy, for example magnetic energy. Embodiments may include a magnet, electromagnet, and/or magnetizable materials, for example, to position the embodiments herein with a magnetic field. Magnetizable materials include ferrite particles, which may or may not be magnetized. The electromagnet may selectively provide a magnetic field with application of current. The magnetic field may emanate from any portion of the embodiments herein. The magnet, electromagnet, and/or magnetizable materials may be mounted on or included in all or any portion of the embodiments herein. A leading end portion may be utilized to position and/or guide the system. The magnetic field may emanate from a portion of the system or from an external source positioned outside the body. The polarity and/or strength of the magnetic field may be increased, decreased, or alternated to controllably guide the system, for example effector 110. Embodiments of the present disclosure may also include any devices and methods disclosed in U.S. Pat. No. 7,320,319, titled “Medicant Delivery System and Method”, U.S. Patent Application Publication No. 2008/0306324, titled “Magnetic Joint Implant”, and U.S. Pat. No. 6,719,765, titled “Magnetic Suturing System and Method”, which are hereby incorporated by reference in their entirety.

Embodiments may include a robotic arm. The robotic arm may include a robotic mechanism. Embodiments may be configured to position and/or guide any portion of the system, for example effector 110. The robotic arm may be utilized to remove tissue, expand, or if this is open channels to enhance drainage. The robotic arm may include a haptic arm. The robotic arm may be automatic, programmed, user controlled, and/or remote controlled. The motion path of the robotic arm may include end points or boundaries, for example, to minimize user error. Embodiments of the present disclosure may also include any devices and methods disclosed in U.S. Patent Application Publication Nos. 2003/0181800, titled “Methods of securing body tissue”, and 2012/0221017, titled “Methods for Robotic Arthroplasty”, which are hereby incorporated by reference in their entirety.

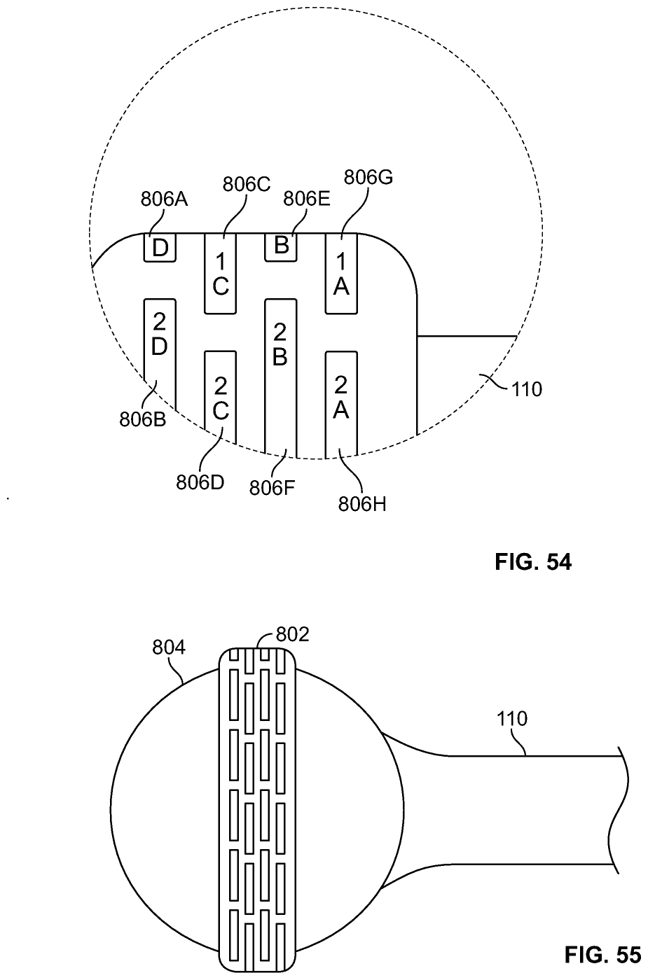

Effector 110 may include foil 802 disposed around all or a portion of expandable portion 804. (FIGS. 53-55). Foil 802 may include a resistive heater with one or more resistive elements 806. (FIG. 53). Expandable portion 804 may include an inflatable device (i.e. balloon, or web) with a contracted condition (FIG. 53) and an expanded condition (FIG. 55). Resistive elements 806A-H may include gold plated ceramic elements wherein the plated element is designed to heat to a temperature sufficient to ablate tissue. (FIG. 54). Effector 110 may include multiplexer 808, for example, to provide a multiplexed time and/or space thermal profile to foil 802. Power delivery rate and/or ability of the body to dissipate energy may be limited. Furthermore, tissue thickness and/or consistency may vary with respect to heat exposure. A temperature feedback system may include sensors integrated into each elements 806, which may generate a thermal profile from the surface of the tissue. Embodiments may be powered with a battery or generator.

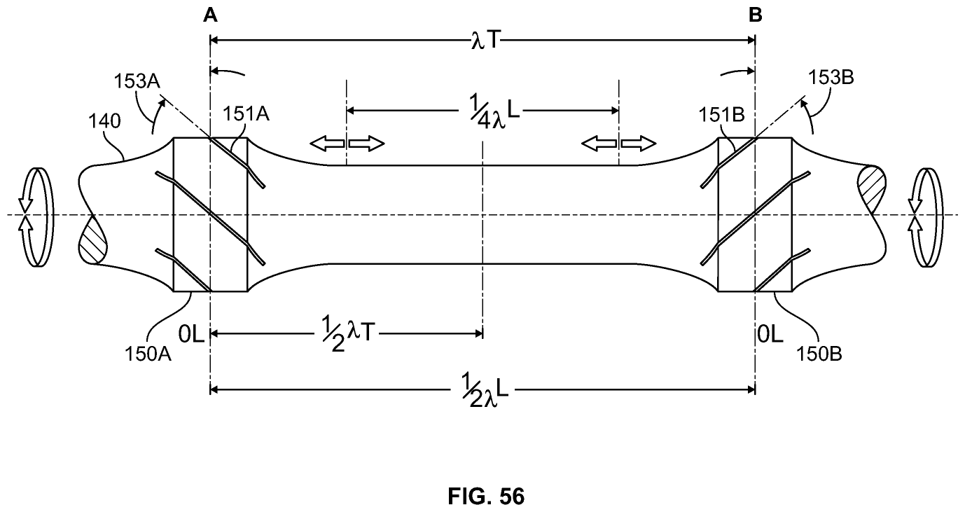

Embodiments may include multiple transformers 150, self-nulling, and/or active dampening. (FIG. 56). Transformer 150 may include one or more slit 151. All or any of slits 151A may be at any angle 153A with respect to the transverse axis of transducer 140, for example about negative 30-60 degrees (negative 45 degrees shown). All or any of slits 151B may be at any angle 153B with respect to the transverse axis of transducer 140, for example about positive 30-60 degrees (positive 45 degrees shown). For example, slits 151A and 151B may be about the same magnitude in opposite directions with respect to the transverse axis of transducer 140, which may null-out torsional motion at one wavelength of extensional motion and one-half wavelength of torsional motion.

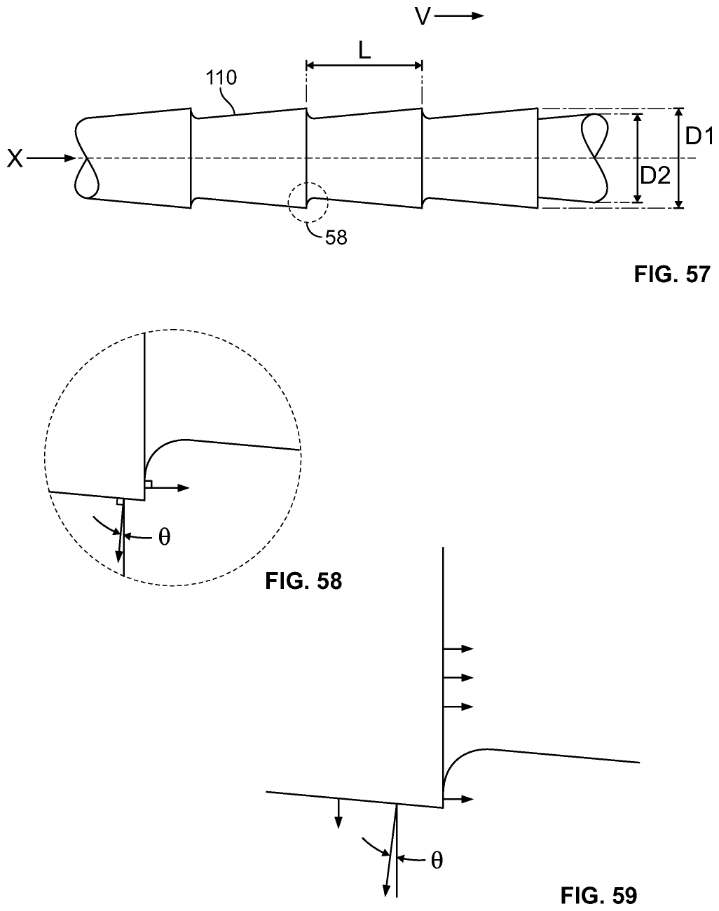

Embodiments may include a streaming wire and/or pump actuator. (FIG. 57-59). Effector 110 may include a plurality of conical steps, for example, to facilitate flow in the direction of the longitudinal axis of effector 110. (FIG. 57). The steps may be conical and/or include a radius adjacent base of each step. (FIG. 58). The surfaces of each step may be angled with respect to the longitudinal axis of effector 110. (FIG. 59). Fluid in contact with a smooth effector may be propelled along the surface of an oscillating surface by way of a phenomenon describe as Stokes oscillating boundary layer for laminar flow. Fluid in contact with the oscillating surface is propelled from the high velocity region towards the low velocity region. A resonating wire that is touching a pool of fluid will lift the fluid along the wire to the first node or zero velocity position. The means by which this is accomplished is due to adhesion of the fluid to the oscillating surface, cohesion of the fluid and surface tension. The fluids may develop a boundary layer and enables the flow. As an example, a wire of 0.020″ in diameter and oscillated at 70,000 Hz extensionally, water can be pumped at 3 cc per min depending of the amplitude of effector 110. Another common way to pump fluid is to impart momentum into a driven impellor or piston. In a similar fashion, effector 110 may have features placed on effector 110 (FIGS. 57-59) to act as momentum transferring surfaces. Effector 110 may possess surfaces in planes substantially normal to the longitudinal axis effector 110. Upon movement of the surfaces in the longitudinal direction, the surfaces compress against the fluid thereby inducing the fluid to move along the longitudinal axis of effector 110. (FIG. 57). The area of each surface is difference between the major and minor diameters, D1 and D2 respectively. The flow generated is a function of the area and velocity of each surface.

Many fluids may be incompressible under compression but may boil if subjected to a vacuum pressure. A differential flow field can be developed across a plane where on one side of the plane fluid is compressed and on the other side the fluid is under cavitation. Effector 110 may oscillate along its longitudinal axis in the x direction. (FIG. 57). A differential potential flow field may be ultrasonically generated across each of the annular surfaces. The flow field may have a localized effect. Turbulence may be created along the edge of the annular surfaces (FIG. 58) and/or along the length of effector 110. The annular plane intersects an opposing conical surface. This surface, when moving, may induce a vacuum pressure on the fluid when the annular plane is compressing the fluid. This vacuum may be developed over the surface of effector 110. While the fluid cavitates due to reduced pressure, the momentum transfer by vacuum may be limited. The reverse effect is developed when the annular plane is moving in the oppose direction.

The surfaces of effector 110 may be configured to adjust flow. By grinding edges into the surface of effector 110, edge flow and/or turbulence may be created in combination with momentum induced streaming. By varying the depth of the ground edge, the length of the reversed cone, the number of edges and their directions, and where they are located relative to the ultrasonic waveform in effector 110, edge flow and streaming direction and intensity may be regulated. This flow can be superimposed with the Stokes boundary layer effect to reduce and increase the flow effects. In addition to the fluid flow effects, the ground edges can act as small blades to carve away bits of solid material. The combined effect of effector 110 eroding the solid material and the ultrasonically induced streaming flow on the surface of effector 110 may erode and/or flush tissue or pathologic material away from the treatment site.

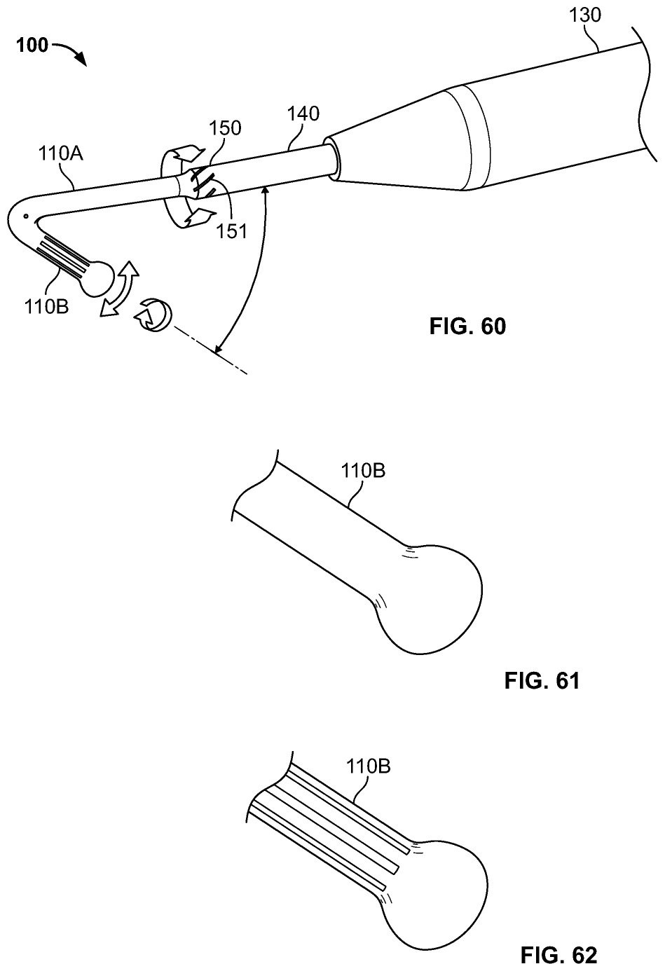

The shape of effector 110 may be configured to navigate around any anatomic curvature of the body. (FIGS. 60-62). For example, the maxillary sinus may require a bend of about 135 degrees. Handpiece 100 may include a substantially straight segment and a substantially bent segment interposed by a node. Transducer 140 and effector portion 110A may comprise the substantially straight segment, for example about 80 millimeters in length. Effector portion 110B may comprise the substantially bent segment, for example about 23 millimeters in length. In use, a multimodal transducer and effector may be tuned such that the bent segment rotates and/or transversely flexes to project ultrasonically and/or radiate the occluded area, for example the maxillary sinus. Effector portion 110B may include a body portion, for example about 2 millimeters in diameter, and a leading end, for example about 3 millimeters in diameter. (FIG. 61). Effector 110 may include surface features and/or be treated to induce motion, flow, friction, and/or erosion. (FIG. 62).

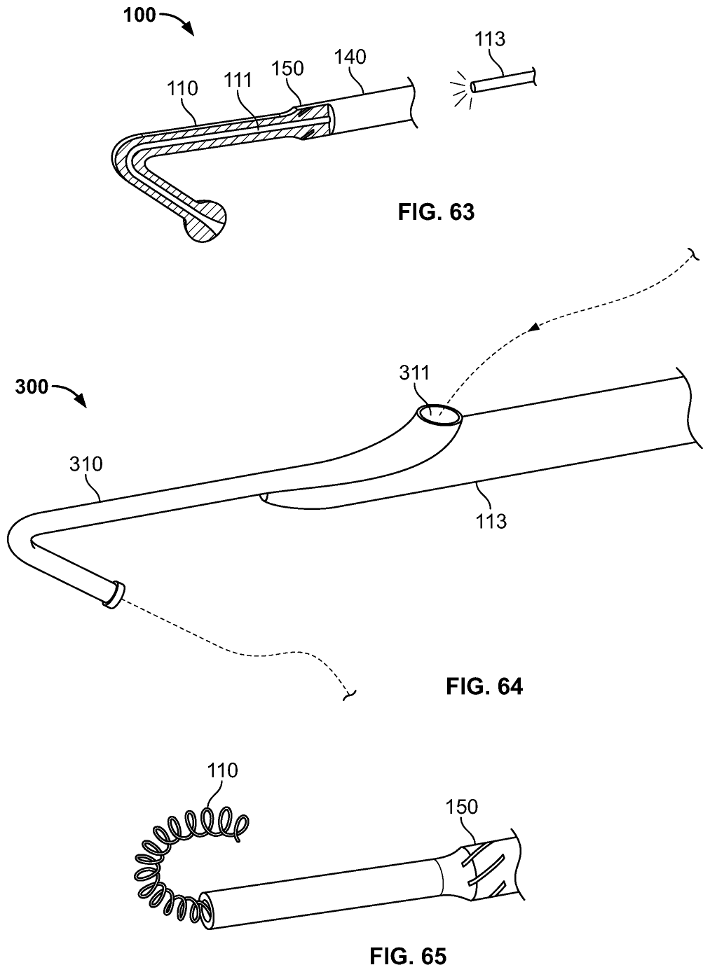

Handpiece 100 and/or cannula assembly 300 may be configured to receive a medical instrument 113. For example, medical instrument 113 may include a light guide (i.e. a fiber optic cable) positionable through all or a portion of passage 111. (FIG. 63). As another example, effector 110 (i.e. flexible wire) may be positioned through all or a portion of passage 311. (FIG. 64). These embodiments may be utilized to maneuver around anatomical curvatures, for example, to access a maxillary sinus.

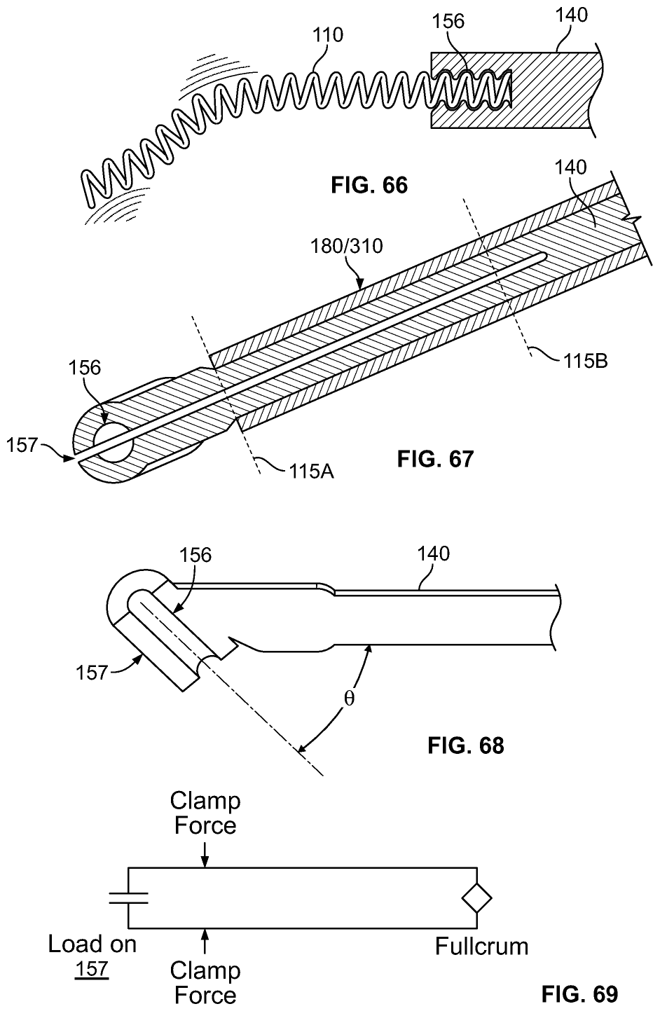

Embodiments may include an impedance mismatch and/or be configured to induce sympathetic or parasitic vibration. (FIG. 65). Effector 110 may have a different mechanical impedance than transducer 140. Embodiments may be configured to decrease the portion of the energy that passes from the transducer 140 into the effector 110, for example by making transducer 140 more rigid and/or effector 110 less rigid. In an embodiment, effector 110 may be a spring, coil, and/or wire. (FIG. 65). In addition, effector 110 may be a softer material than transducer 140, for example effector 110 may be a polymer, a metal with a lower modulus of elasticity, composite, or any combination thereof. In another embodiment, attachment 156 between effector 110 and transducer 140 may provide the impedance mismatch, for example, with effector 110 being a coil at its proximal end at the junction and more solid toward its distal portion. (FIG. 66).

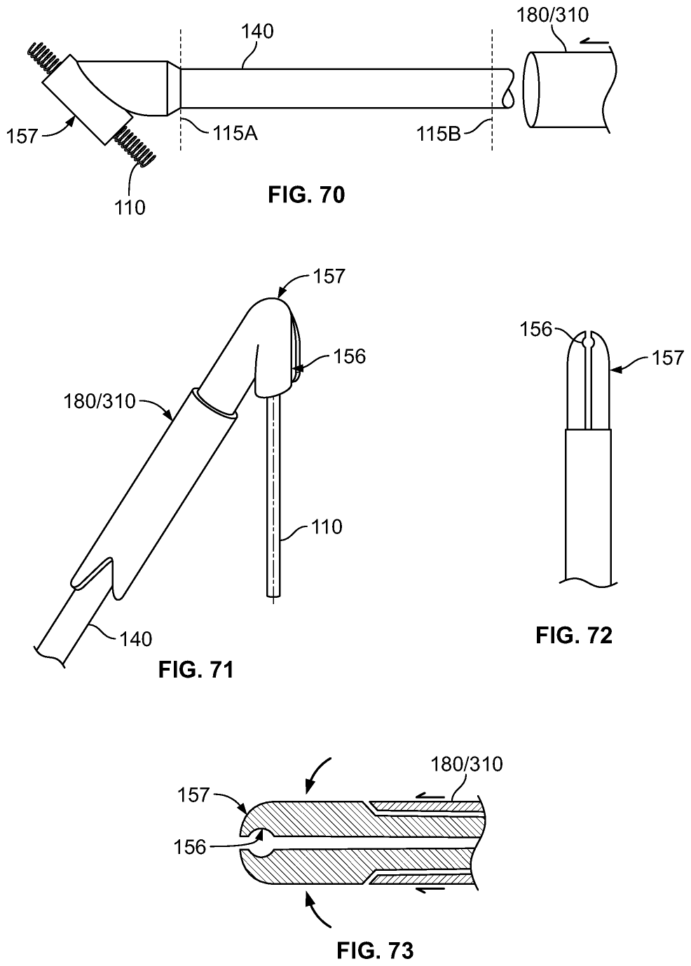

Transducer 140 may be configured to releasably grasp effector 110. (FIGS. 67-73). An effector may be configured to releasably grasp another effector 110. Transducer 140 may be configured with one or more vibrational nodes 115A and/or nodes 115B. Attachment 156 may include graspers 157. Grasper 157 may include a slit to receive effector 110, an inner surface contoured the exterior surface of effector 110, and/or may be angled with respect to the longitudinal axis of transducer 140, for example about 45 degrees. (FIG. 68). Grasper 157 may be configured to bend effector 110. (FIGS. 71-72). Upon application of a force in the longitudinal axis of cannula 180/310, cannula 180/310 may be urged over rib 111 of transducer 140 thereby applying a clamp force to radially compress graspers 157. (FIGS. 69 and 73). Transducer 140 may be releasably coupled with cannula 180/310. (FIG. 70). Rib 111 of transducer 140 and leading end of cannula 180/310 may include conical surfaces, for example, to facilitate radial compression of graspers 157. The radial compression of the node 115A may minimize heating in the cannula 180/310, for example, because cannula 180/310 may only be excited in a radial direction due to Poisson effect in the longitudinal node. The cannula 180/310 may be comprised of any materials disclosed herein, for example any metal, plastic or ceramic.

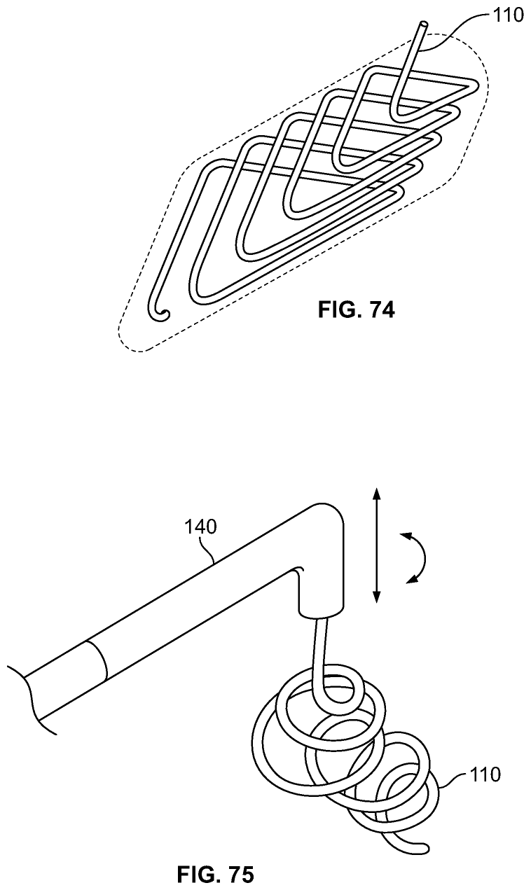

Effector 110 may be shaped to a specific cavity of the body, for example a sinus cavity. (FIG. 74-75). One or more effectors 110 may formed and/or assembled to contour the surface of the cavity. Effector 110 may be assembled such that when relaxed it takes the sinus shape, then radiates vibration and/or erodes pathological materials along its length upon excitation. Embodiments may use vibratory energy to mechanically agitate therapeutic substances.

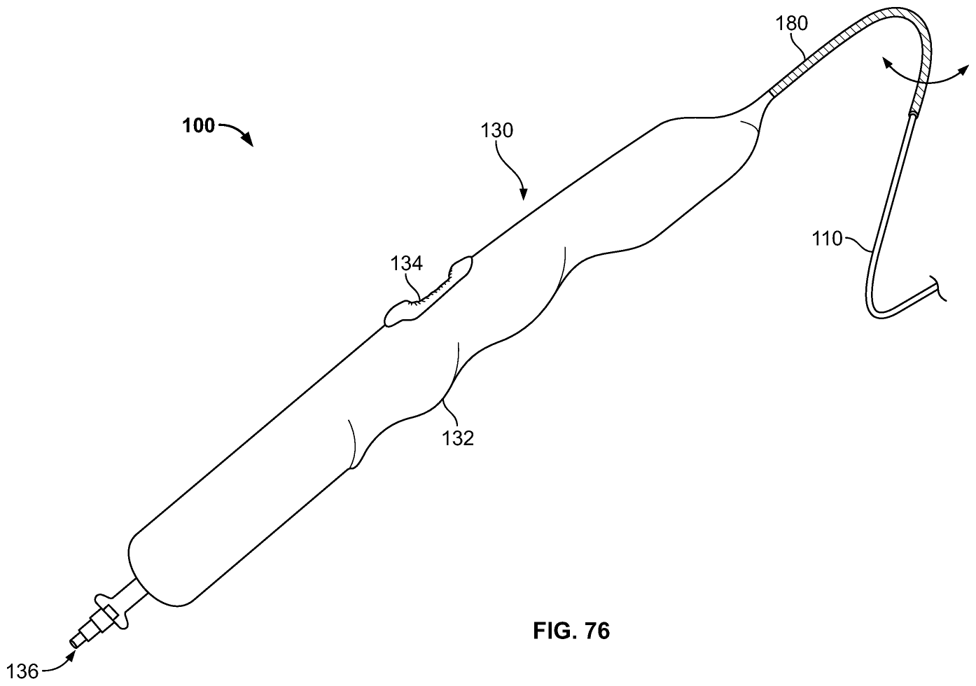

Handpiece 100 may include handle 130, grip 132, switch 134, connector 136, and/or cannula 180. (FIG. 76). Grip 132 may be contoured to facilitate user grip. Switch 132 may be configured to activate, adjust, and/or de-active the operation of handpiece 100. Connector 136 may include a lever fitting. Connector 136 may be configured to introduce therapeutic substances and/or may be configured to provide suction to remove the same or pathologic material.

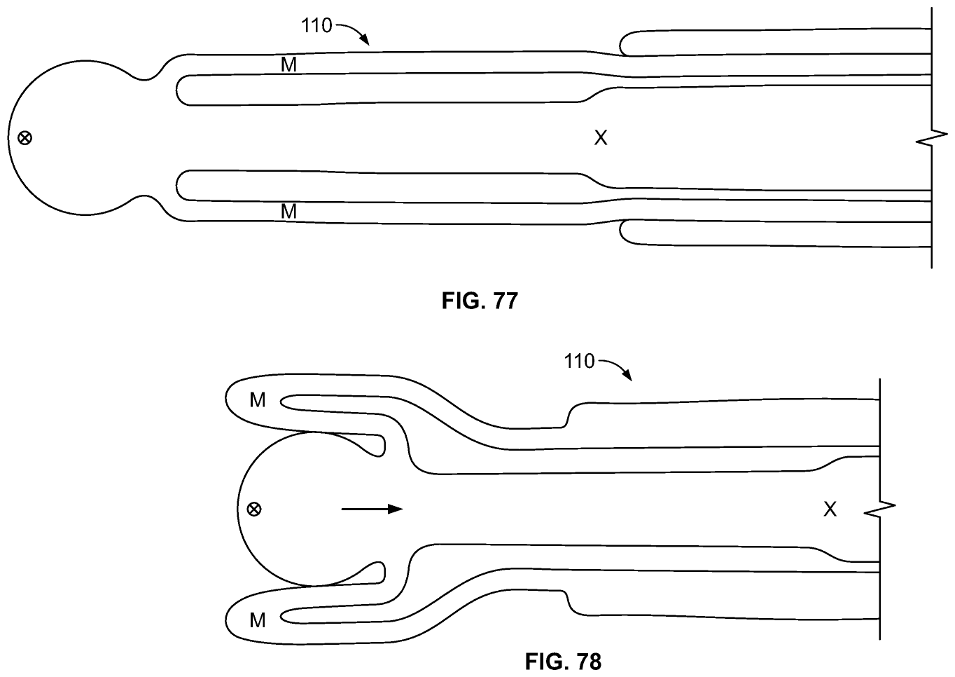

Effector 110 may also be configured as a dilator, for example with or without a mechanical or vibratory energy. (FIGS. 77-78). All or any portion of effector 110 may include any material disclosed herein, for example a flexible material such as silicone, latex, or nitrile. Effector 110 may be positioned in an elongate configuration. (FIG. 77). An inner portion of effector 110 may be longitudinally contracted relative an outer portion of effector 110 thereby reducing the length of effector 110 and/or radially expanding the outer portion of effector 110. In use, effector 110 may be introduced into a passage then the inner portion may be urged backward relative to the outer sleeve thereby dilating the passage. Expansion of the outer portion of effector 110 from an initial diameter (i.e. about 2-4 millimeters, preferably about 3 millimeters) to a second diameter (i.e. about 5-15 millimeters, preferably about 6 millimeters) may thereby dilate a portion of the body. The inner portion may be held extensionally by a compression spring held in place by a trigger. Once the trigger is actuated the inner portion would retract thereby expanding the system to a preset diameter. The inner portion may be oblong or cylindrical. The inner portion may then be further withdrawn, allowing the “M” diameter to collapse to a diameter, smaller than an introducer and/or passage thereby facilitating its release.

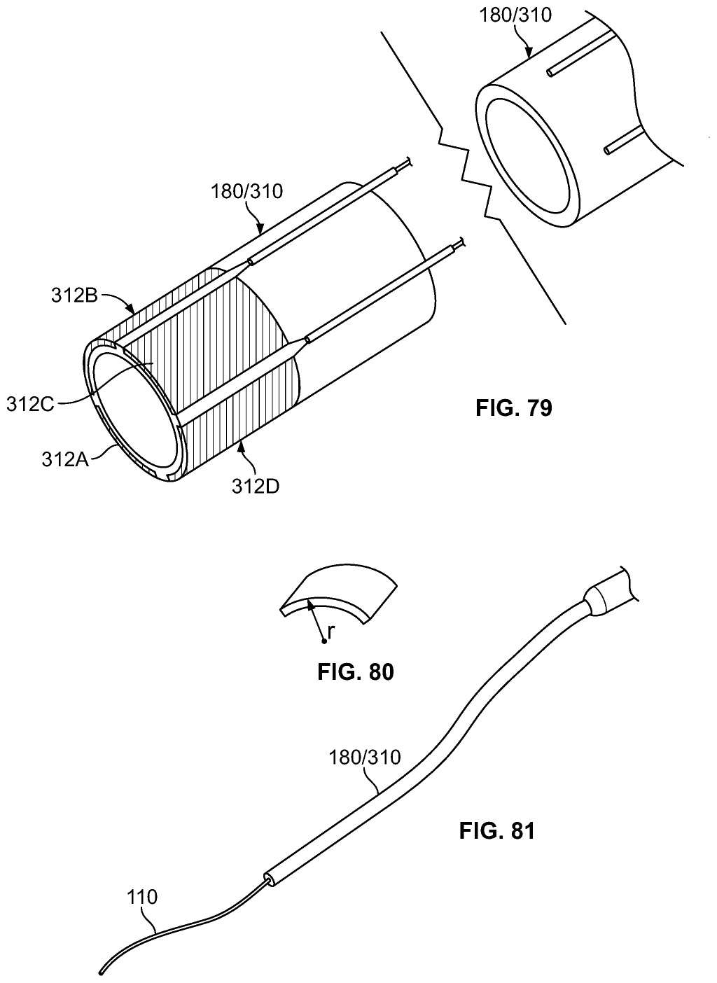

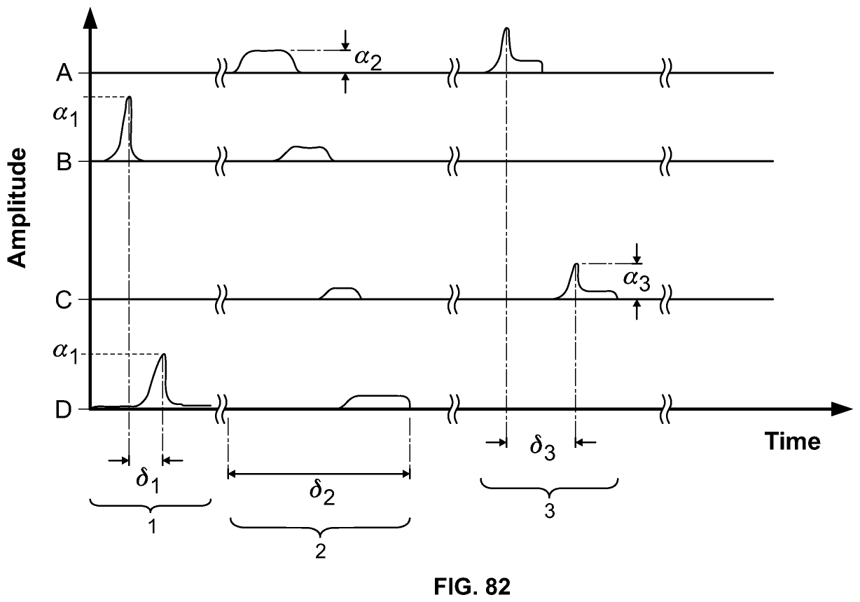

Embodiments may include sensor 312, for example a hydrophone with a piezoceramic array. (FIG. 79-81). Sensor 312 may include any number of piezoceramic elements 312A-D, for example 4 (shown) or 8. (FIG. 79). The array may be configured so that movement and/or contact of the effector 110 may induce a voltage in one of the piezoceramics in the array. Each successive movement and/or contact of the effector 110 would induce a voltage in a ceramic. The output from the ceramic may indicate the location of the effector 110 impact relative to the circumference of the distal portion of the cannula 180/310. The location, time and intensity information from the piezoceramic array may be interpreted by a detecting circuit to describe the spatial information about the trajectory of the movement of effector 110. Referring to FIG. 82, scenario A indicates transverse vibration with a period indicated by 2×delta 1. Similarly, scenario B indicates rotational motion in the wire with a period indicated by delta 2, because the sensor is active at lower intensities for longer time periods. Effector 110 behavior may indicate a multitude of outputs on the piezoceramic arrays. Crystals in cannula 180/310 may be used as a hydrophone to measure the acoustic output of wire for input to the control system. In use, the output of sensor 312 indicates the physical motion of effector 110 during excitation. This output may be utilized as an input for an electric generator having a feedback control algorithm. The amplitude of the output from elements 312A-D due to the movement and/or contact of the effector 110 at distal portion of catheter 180/310 varies with the effector 110 output power. (FIG. 82).

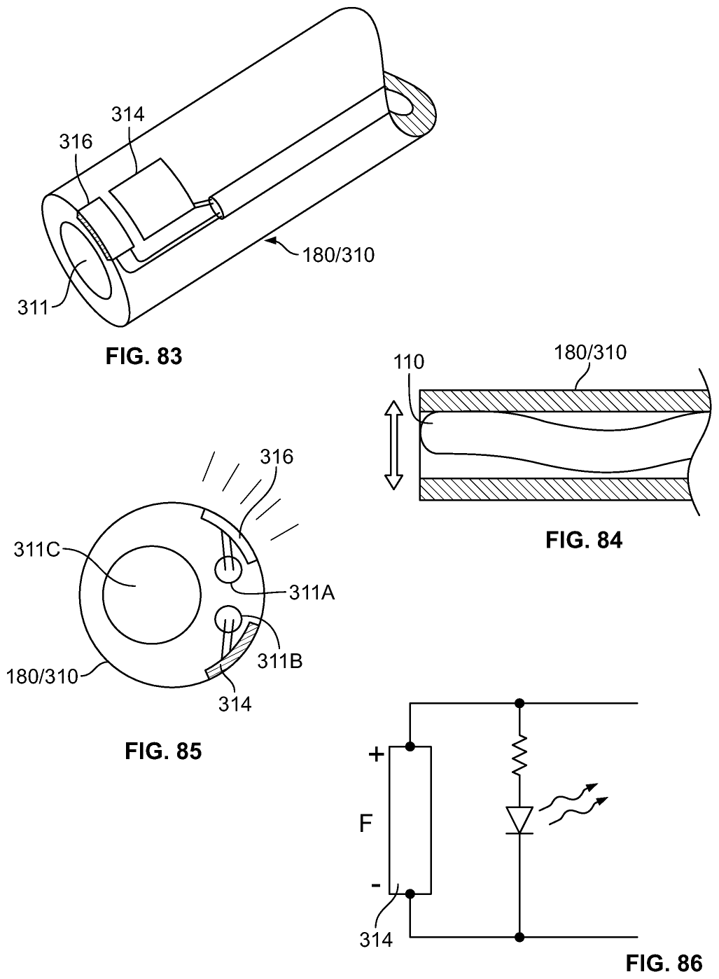

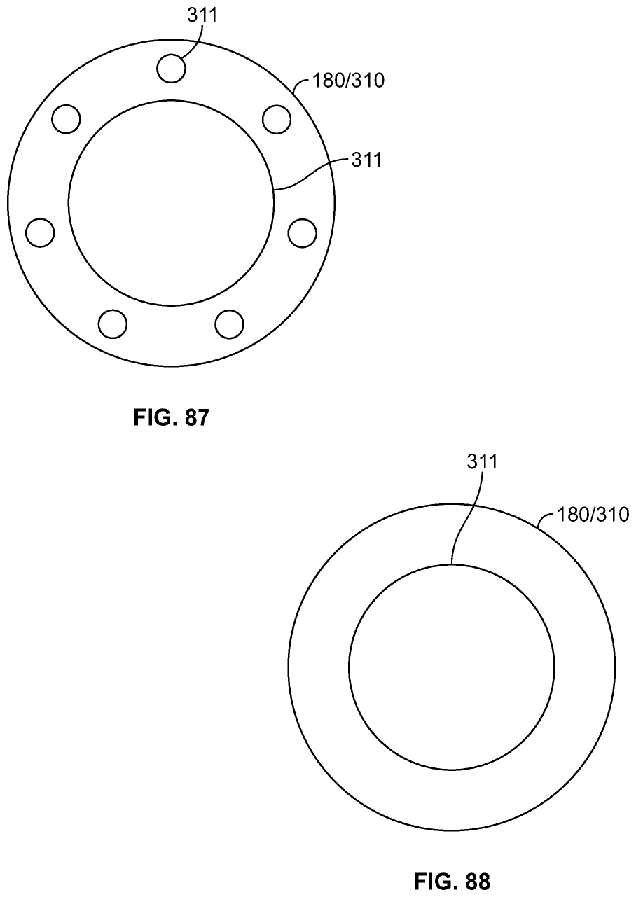

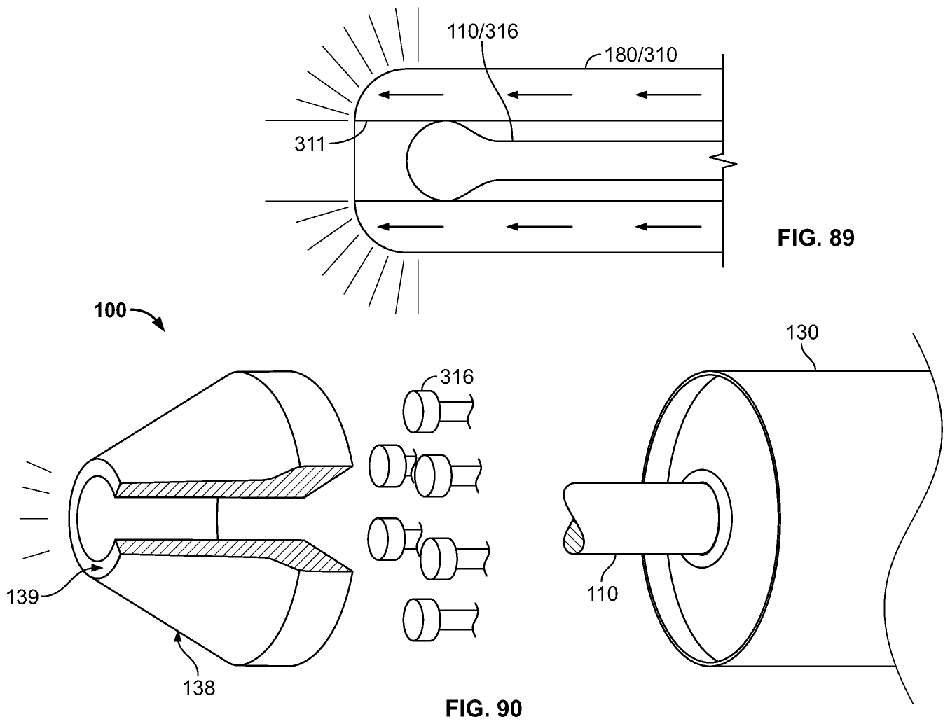

Cannula 180/310 may provide illumination and/or include one or more lights 316. (FIGS. 83-90). Lights 316 may be configured to provide vision, navigation and/or color indication. The light (i.e. UVC or other ultraviolet wavelengths) may emit a wavelength that can help reduce pathologic materials (i.e. bacteria). Cannula 180/310 may include a passage 311, piezoceramic 314, light 316. Light 316 may include a LED or any other light disclosed herein or known in the art. Light 316 that may be illuminated with power directly from an energy source without requiring power on effector 110 and/or powered from energy source 120. (FIG. 83). Light 316 may be powered by the impact of the effector 110 against cannula 180/310 (FIG. 84). Cannula 180/310 may include piezoceramic 314 (FIGS. 85-87), wire lead passage 311 A-B (FIG. 85), and/or 316 on (FIG. 83) or beneath (FIG. 85) the surface of cannula 180/310. Cannula 180/310 may include multiple passages 311 A-C, for example, to receive effector 110, light 316 leads, and/or other objects or materials. Lead passage 311 A-B may connect piezoceramic 314 with light 316. Sensor passage 311 A-B may connect piezoceramic 314 with another sensor. Embodiments may include a light emitting device (i.e. LED) and a circuit powered by piezoelectric ceramic. (FIG. 86). Cannula 180/310 may be illuminated with a conductor embedded in or on cannula 180/310, for example, including a urethane or polypropylene material. (FIGS. 87 and 88). Alternatively, cannula 180/310 may be illuminated with a light guiding material extrusion forming a lumen and light guide conduit, for example, including an acrylic material. (FIGS. 88 and 89). Cannula 180/310 may be illuminated with an array of lights 316 disposed within diffuser 138 of handpiece 100. (FIG. 90). Diffuser 138 may include light surface 139 that may be configured to engage the lighting features or passages of cannula 180/310. Diffuser 138 may include an interface for an irrigation device and/or handle 130. The fluid used in irrigation may also be used as the light conducting medium.

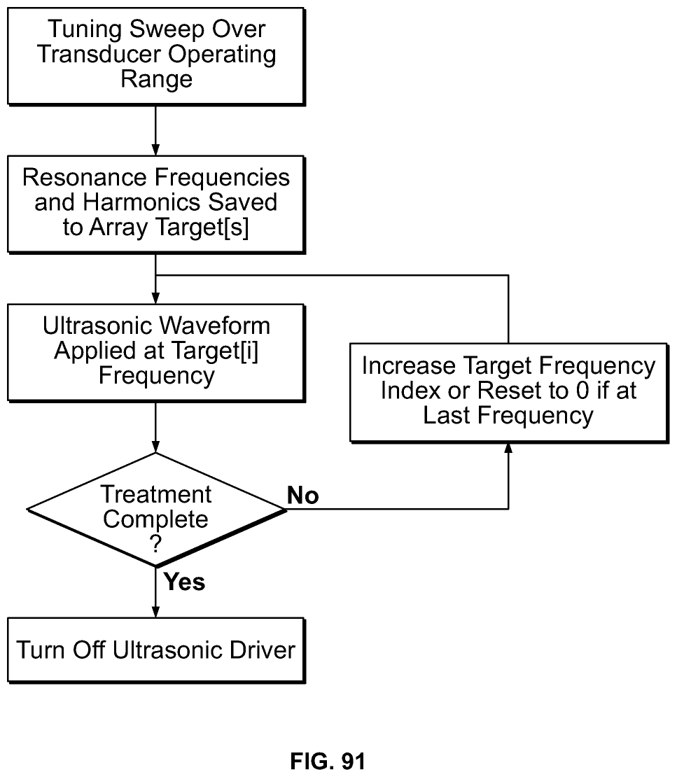

Embodiments may include control systems and methods, for example, related to vibratory energy. (FIGS. 91-94). Embodiments may include a vibratory energy system including a control system, signal generator, and amplifier connected to a transducer, for example a piezoelectric stack. The transducer may be any other type disclosed herein, for example magnetostrictive.

In an embodiment, the control system may have a fixed frequency, may be analog, and/or may have limited or no microcontroller interaction. The drive system may be configured to maintain the desired system resonance by compensating for temperature and pressure. In a system where minimal software dependency is needed the target frequency of the transducer can be pre-set. During assembly, the resonant frequency of the drive system may be determined and set using a potentiometer, for example, on a printed circuit board of the control system. The target frequency may also be stored on a potentiometer on the transducer assembly or hand piece. In this embodiment the value of the potentiometer will be used to set the frequency of an oscillator when the system is activated. The drive and/or control system may be disposable. Alternatively, the control system may be digital and/or include microcontroller interaction.

Embodiments may be software controlled. The software may be configured to perform a tuning sweep of an expected range of the resonance of the transducer. This may allow for compensation for changes in resonance or frequency response, for example, from pressure, temperature, and/or manufacturing issues. The resonance of the expected range may detect parallel and/or series resonances, for example, using impedance or phase to determine resonance. Embodiments may be process the tune information to determine the optimal drive frequency and power for the transducer. Embodiments may be configured to detect the harmonics of the transducer during a tuning sweep as well as the resonant frequency, for example the transducer 140 and effector 110. The frequencies of both the resonance and harmonics may be saved for use in the control algorithm.

Embodiments of the control system may include a frequency hopping algorithm. (FIG. 91). A tuning sweep may be performed of the expected operating range of the transducer. The series and/or parallel resonances and/or harmonics detected from the tuning sweep may be stored, for example in a memory device. While driving the transducer, the drive frequency may be changed between the detected points during the tuning sweep. The drive frequency may be cycled sequentially, pseudo-randomly, or using any pattern to achieve the desired treatment results.

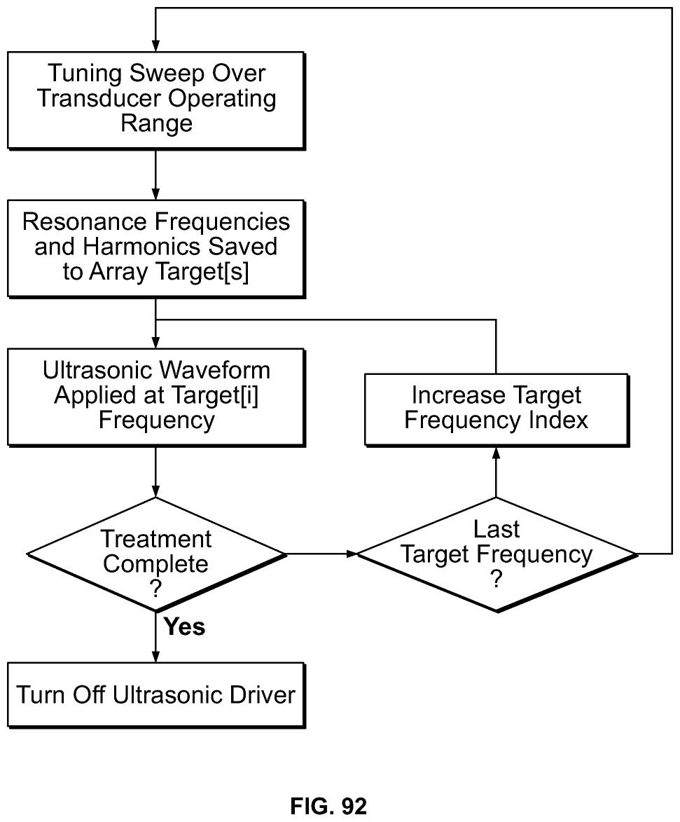

Embodiments may include cyclical frequency hopping, which may be configured to dynamically detect changes to the resonance and/or harmonics during treatment. (FIG. 92). An initial tuning sweep may detect the series and/or parallel resonance and harmonics. While driving the transducer, the drive frequency may be changed between the detected points during the tuning sweep. The drive frequency may be cycled sequentially, pseudo-randomly, or using any pattern to achieve the desired treatment results. After the detected frequencies have been applied, the process may repeat until the desired treatment is complete.

Embodiments may include control systems configured to avoid harmonics that may not produce desired therapeutic results. This may include sensors in the hand piece assembly to monitor the behavior of the effector 110. (FIGS. 79 and 83).

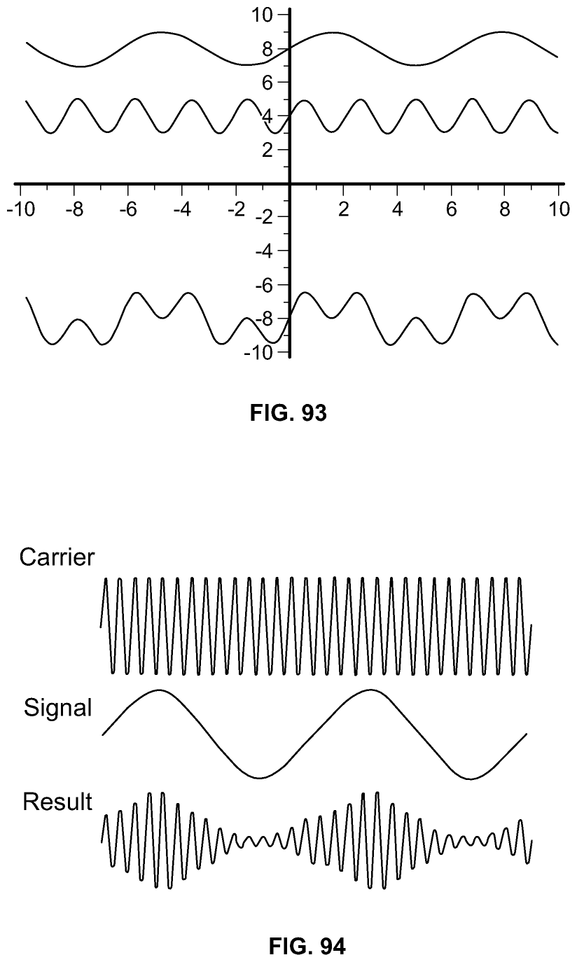

Another embodiment may include a mixed-frequency drive instead of a single frequency drive. A tuning sweep may be done over the expected operating range of the transducer, for example, to detect and store series and/or parallel resonance and harmonics. As an example with reference to FIG. 93, the resonance (i.e. top wave shown) may be combined with the harmonic (i.e. middle wave shown) to get the drive signal (i.e. bottom wave shown) in an attempt to excite multiple operational modes of the transducer. Multiple harmonics may be combined with the drive signal, or the harmonics may be cycled as described above for frequency hopping.

In another embodiment to attempt to excite multiple operational modes of the transducer, the control system may be configured for amplitude modulation. A tuning sweep may be performed of the expected operating range of the transducer. The series and/or parallel resonance and any harmonics may be stored. As an example with reference to FIG. 94, control systems may utilize a harmonic frequency signal sufficiently above the resonance frequency as a carrier signal (i.e. top wave shown) and modulated the harmonic frequency signal (i.e. middle wave shown) to produce a drive signal (i.e. bottom wave shown). If multiple harmonics were found, the harmonic frequencies may be cycled through as the carrier signal, or the highest harmonics could be used as the carrier signal and/or the harmonic frequency signal may be varied between the other detected harmonics and resonance for the drive signal.

Embodiments may include modulation of output energy for the control system. Output energy may be modulated using voltage, power, and/or current. For example, the drive voltage may be kept at a steady average or RMS output. This may provide limited control of the output power, because the current and/or power may vary with changes to the impedance due to loading of the system. As a further embodiment, the drive voltage may be modulated to keep the power output constant. This may provide consistent power output, but may become unstable if the system loses resonance and the phase approaches −90 degrees. Embodiments may monitor the phase of the drive system during treatment and reinitiate a tuning sweep to compensate for shifts in the resonances which are significant enough to make the control system unstable. After the adjusted resonances are found, the system would continue until the treatment is complete. In another example, the drive voltage may be modulated to keep the output current constant. This may be stable and keep the transducer displacement consistent.

The system may be configured to terminate treatment automatically or manually. An activation switch (i.e. footswitch or push button) may be used to continue treatment until the switch is released. The system may be configured to continue until a target power is delivered or a predetermined time has elapsed. The system may use the release of the activation switch to override automatic termination as safety feature.

In addition sinus and/or nasal applications, embodiments of HIFU may be used for applications related to transdermal treatment or prevention of biofilm infections on implants or any cavity, vessel, duct, passage, joint, bone, muscle, ligament, tendon, cartilage, capsule, organ, skin, nerve, or any other body part.

One embodiment of a transdermal treatment method for both treatment and/or prevention would be to place a transducer on the skin surface over the area where the treatment is to be targeted. An ultrasonic signal would then be generated at a frequency based on the depth of the targeted area. It is thought this frequency could be between 20 kHz and 1 GHz. Multiple transducers could be placed on the skin surface and aimed at the treatment area. The use of multiple transducers could allow for lower intensity waveforms on the skin surface to intersect at the treatment area.

In another embodiment transducer arrays could be used instead of single transducers on each treatment area. These transducers arrays could be placed near a treatment area and the ultrasonic beam could be steered to focus on the treatment area. The ultrasonic frequency between 20 kHz and gHz would be picked based on the treatment depth and multiple transducer arrays could be used.

The focusing of the transducer array or single transducers could be done using external navigation tools such as MRI, ultrasound, or X-ray. In another embodiment, ultrasonic imaging could be integrated in to the treatment transducer to reduce the amount of steps needed for focusing the beam.

Another embodiment may include an effector including a transducer in a probe or an end effector that is inserted intracorporeally at the treatment area. The probe may be swallowed and/or positioned remotely using a wired or wireless connection. An ultrasonic signal with a frequency between 20 kHz and 1 GHz may be sent to the effector, for example wirelessly or using a direct connection. The output from the probe or end effector could be used to physically interact or to produce sound waves that interact with the pathologic materials (i.e. biofilm). Other embodiments may include the use of transdermal and/or intracorporeal ultrasound. Dual modes, different waveforms, and/or modes of operation may be applied to the effector. The dual modes could also be superimposed to create areas with higher and lower intensities waveforms. Embodiments may be implemented with other energy sources including electroshock wave, RF, microwave, and/or any energy disclosed herein or known in the art. One, two or more transducers may be configured to produce multiple energy types.

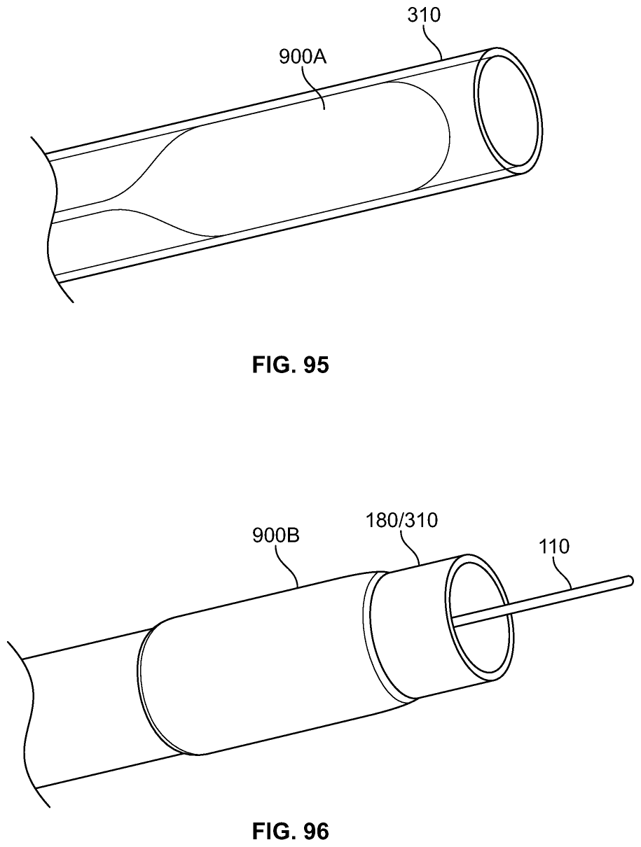

Embodiments may include expandable access devices, for example balloon 900. (FIGS. 95-96). Balloon 900 may be configured to deform tissue and/or position effector 110. Balloon 900 may be configured to apply pressure to a portion of the body, for example a nasal cavity. Balloon 900A may be configured to be positionable in cannula 310 and/or radially expand cannula 310 against tissue. (FIG. 95). Balloon 900B may be configured to be positionable over cannula 300B and/or directly expand tissue. (FIG. 96).

Additional embodiments may be configured to remove pathologic material or resist formation of pathologic material, for example biofilm on tissues and/or implants. An effector may be configured to focus energy on the pathologic material or the surface underlying the unwanted material to remove or resist formation of pathologic material (i.e. biofilm). The energy may be applied to target the material characteristics of the pathologic material or the surface underlying the pathologic material. Targeted material characteristics may include mechanical resonance, electrical resonance, density, modulus of elasticity, and/or any thermal, chemical or molecular properties. For example, the modulus of elasticity may be different for each of soft tissue, hard tissue, unwanted material, biofilm and/or implant. Embodiments may be configured to target the material characteristics (i.e. modulus of elasticity) of where unwanted material formation is undesirable. For example, an effector may be configured to the modulus of elasticity of pathologic material (i.e. biofilm) but not healthy tissue (i.e. bone), thereby allowing effector 110 to remove or resist formation of pathologic material while preserving healthy tissue. As another example, embodiments may be configured to target the thermal properties of pathologic material, for example to thermally breakup enzyme cells. Additional embodiments may apply a secondary energy (i.e. another type of vibratory energy or RF energy), for example, to target a different tissue and/or implant with different material characteristics.

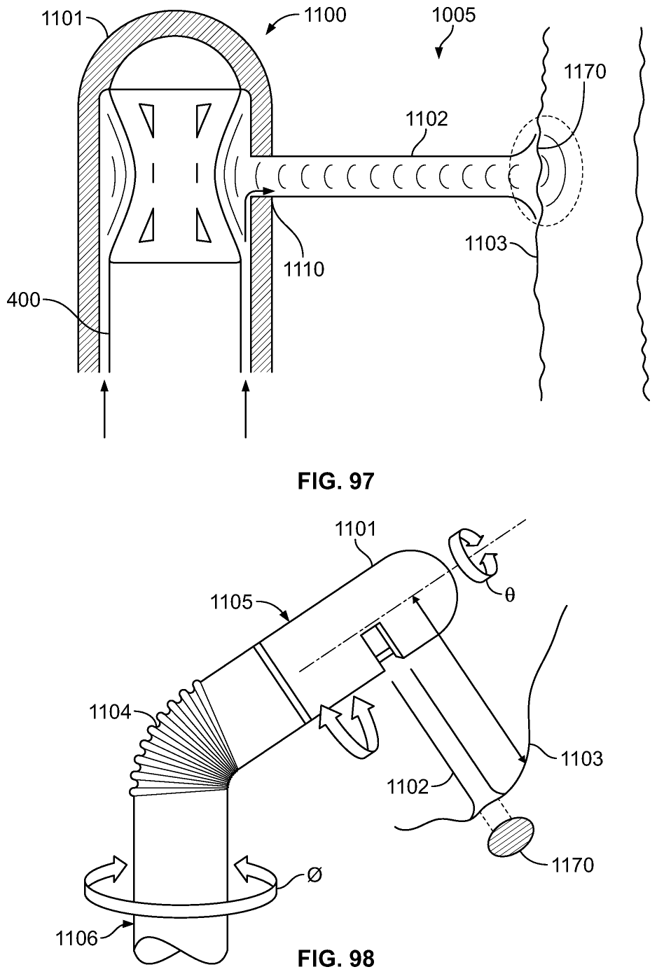

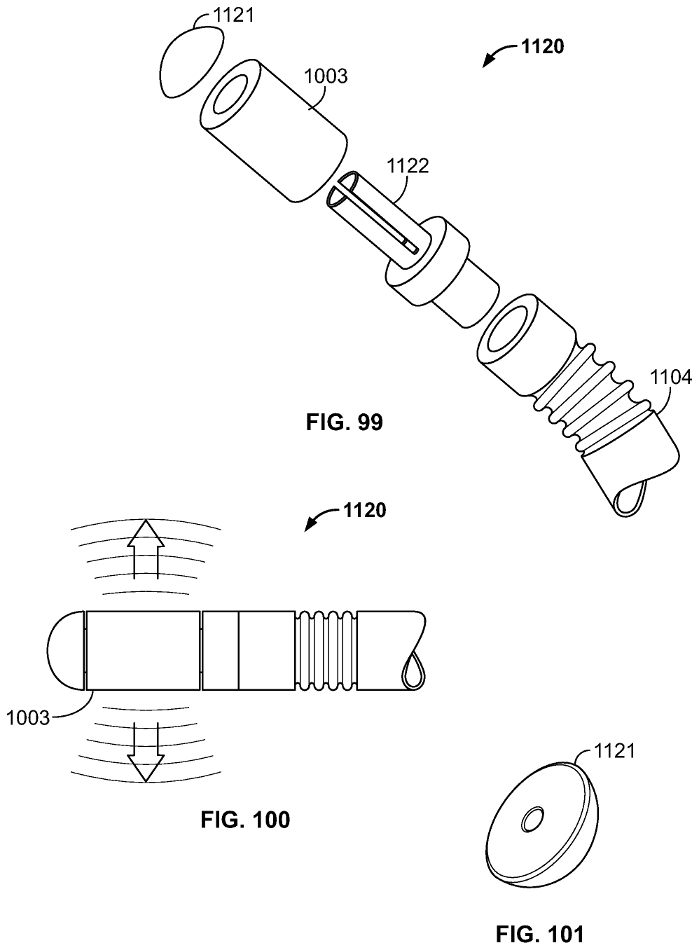

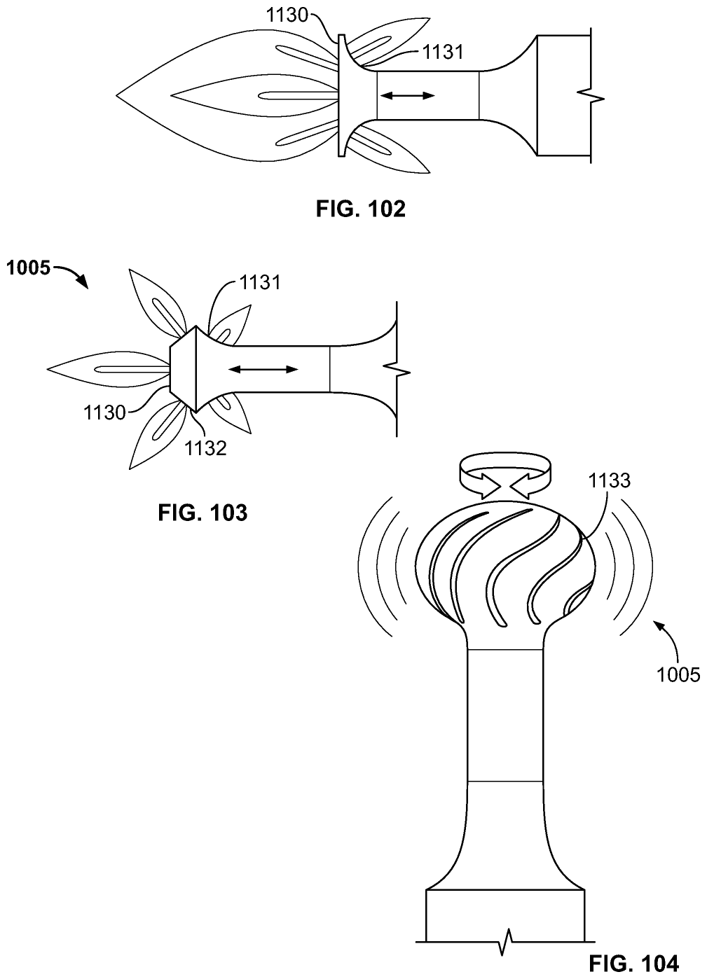

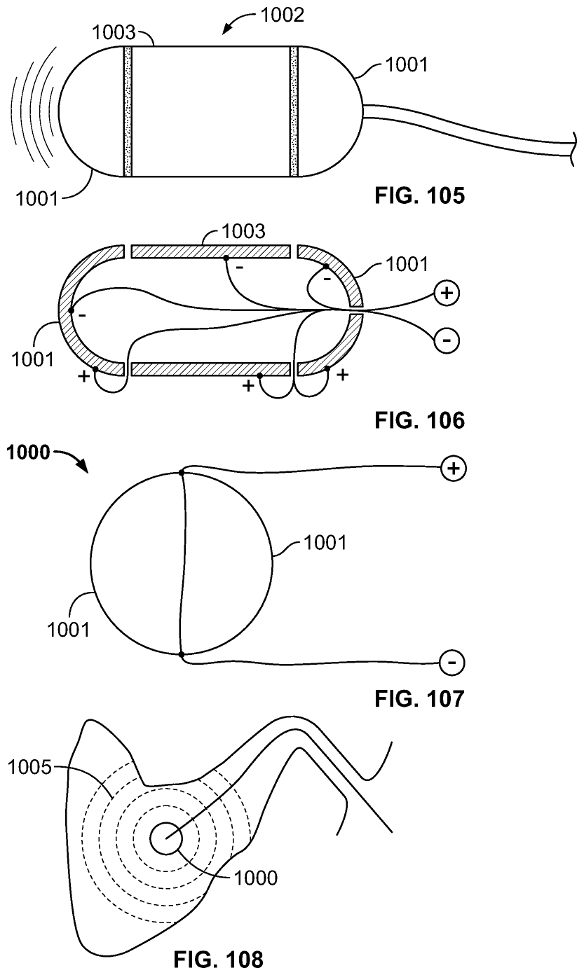

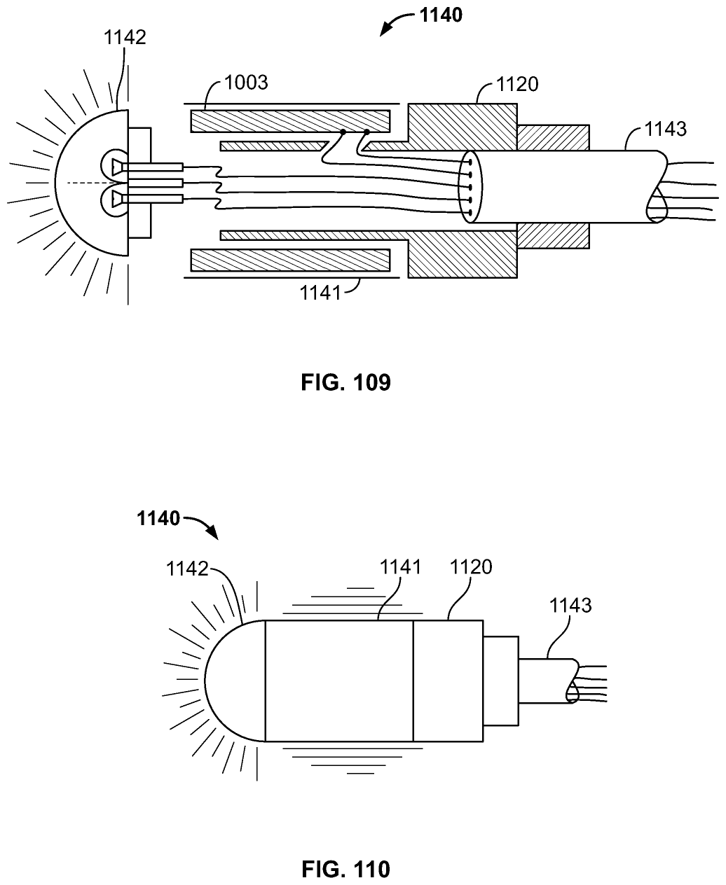

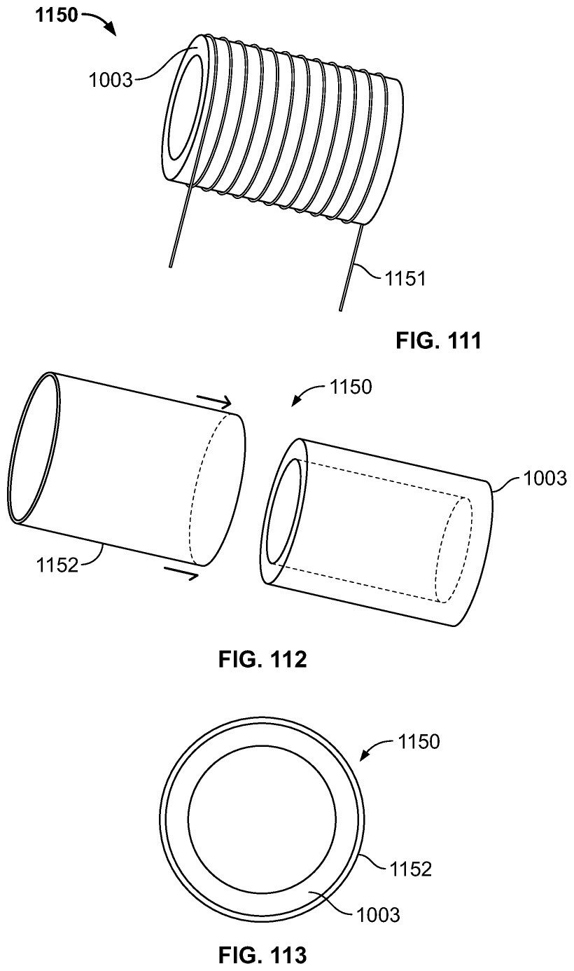

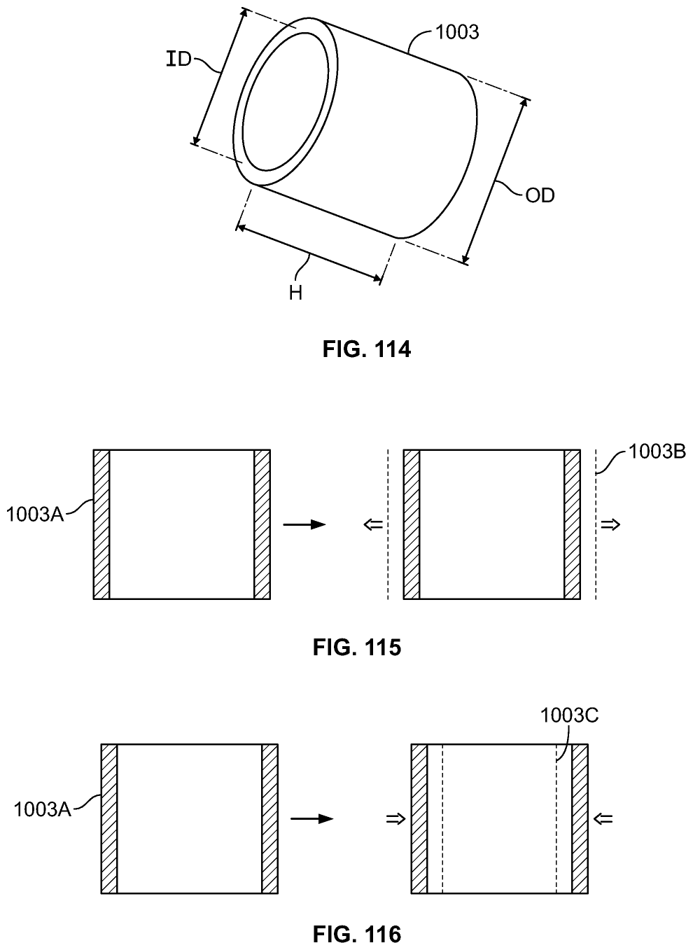

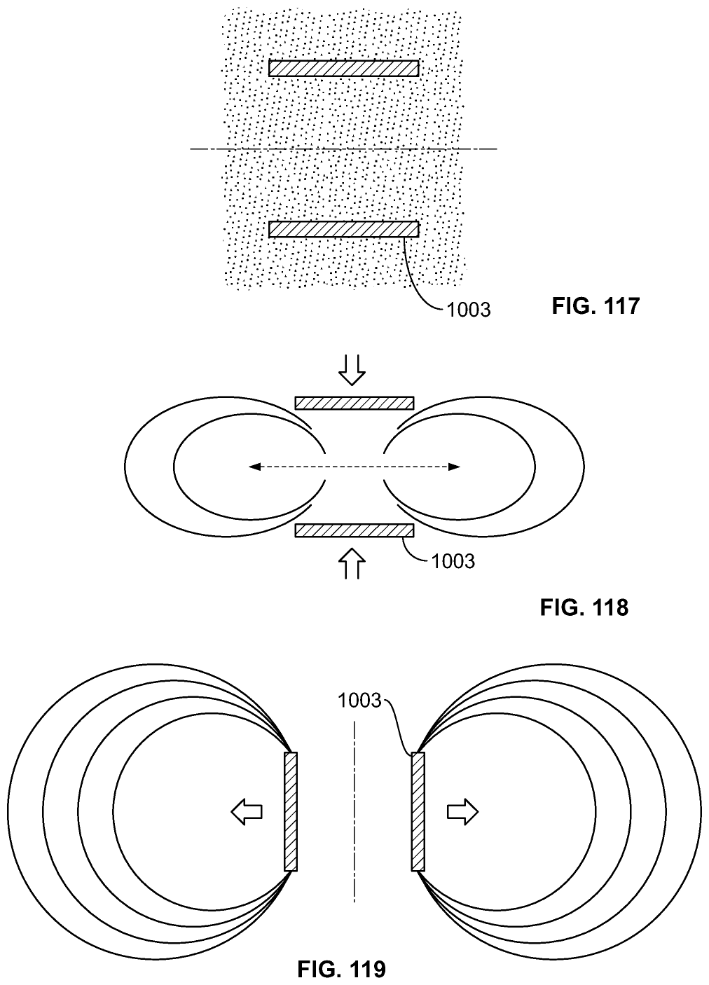

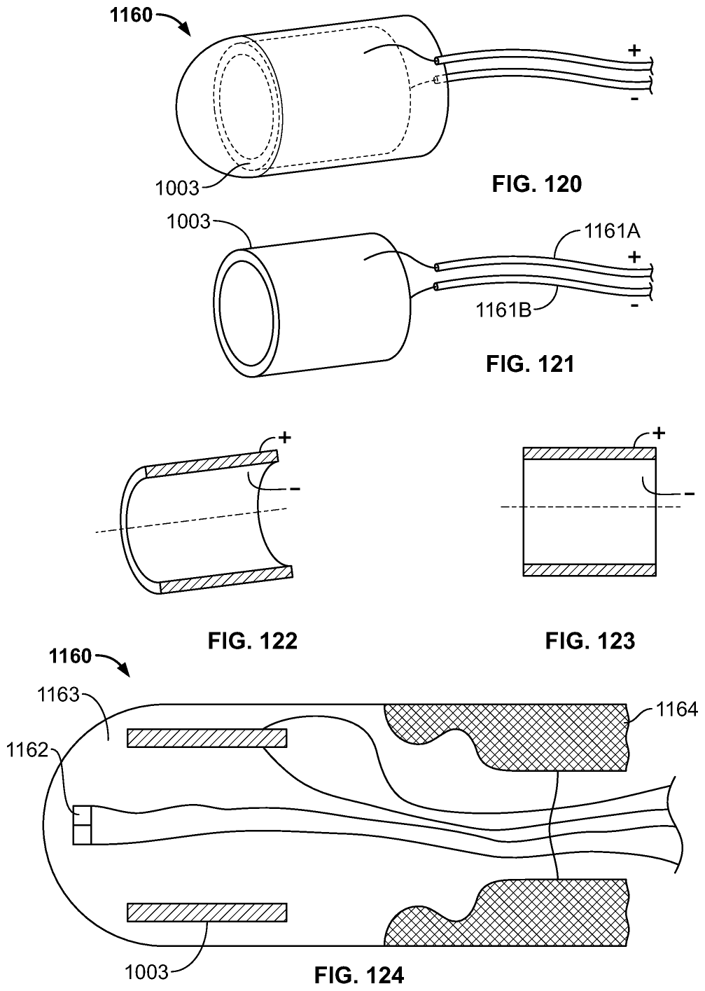

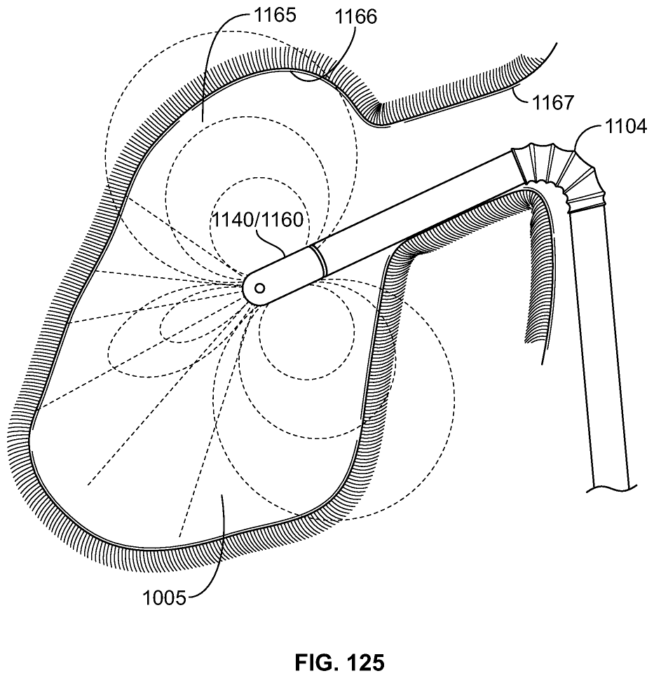

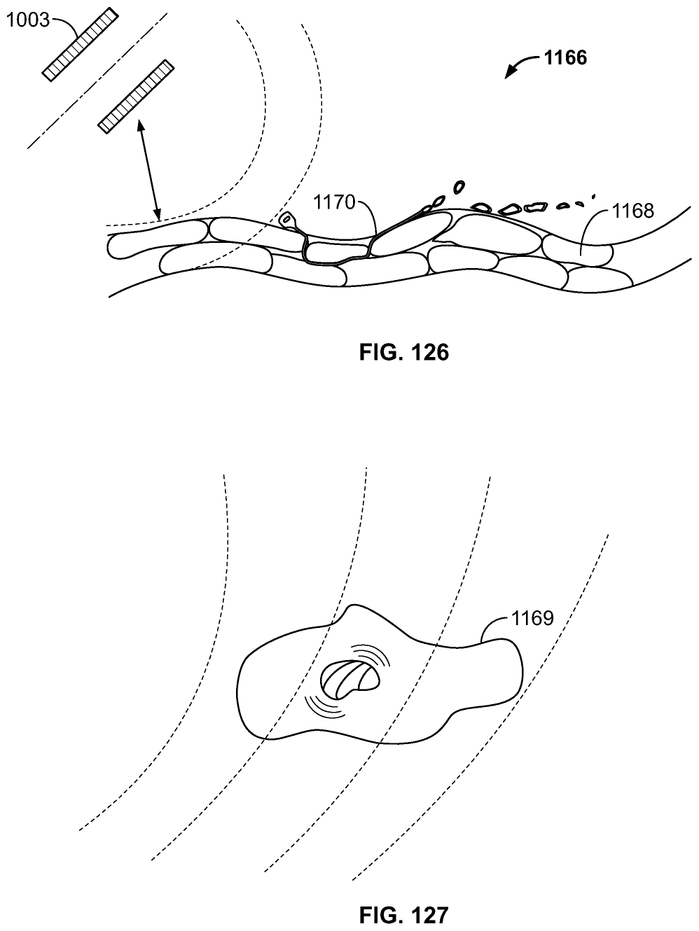

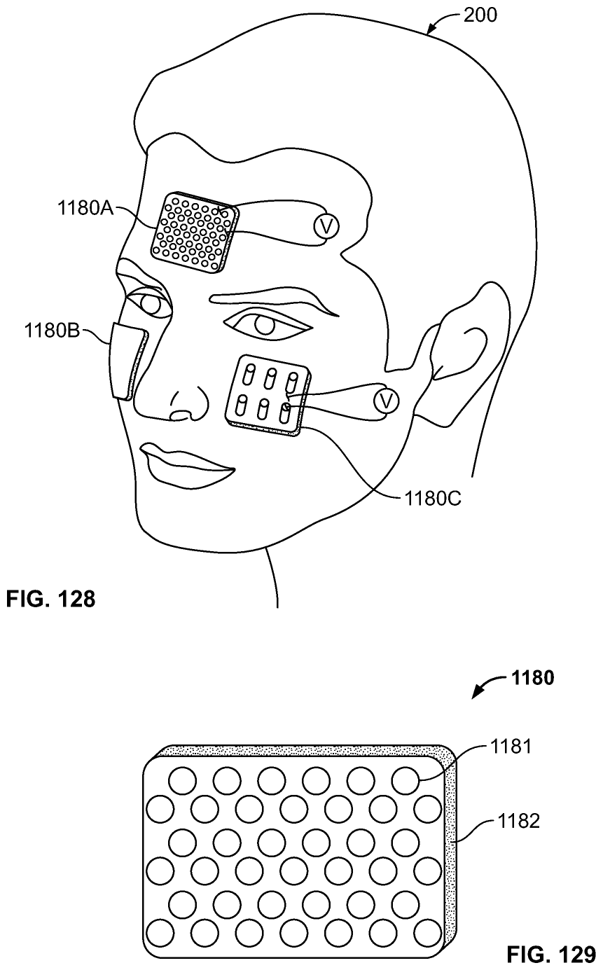

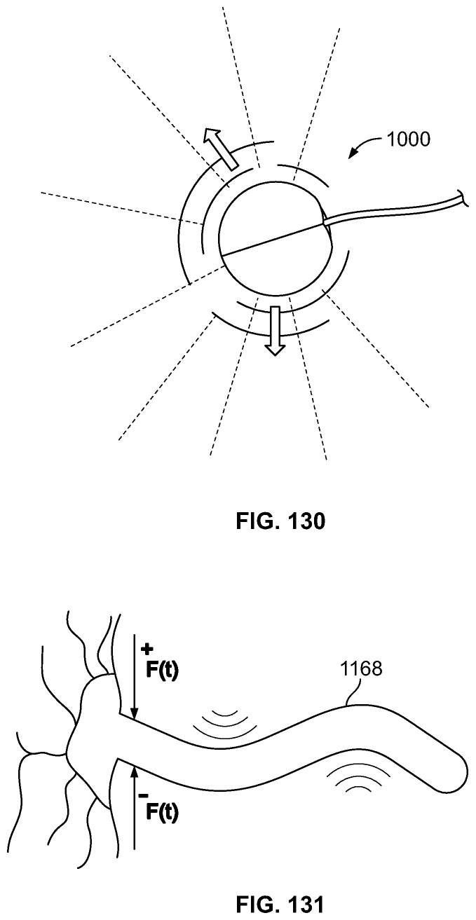

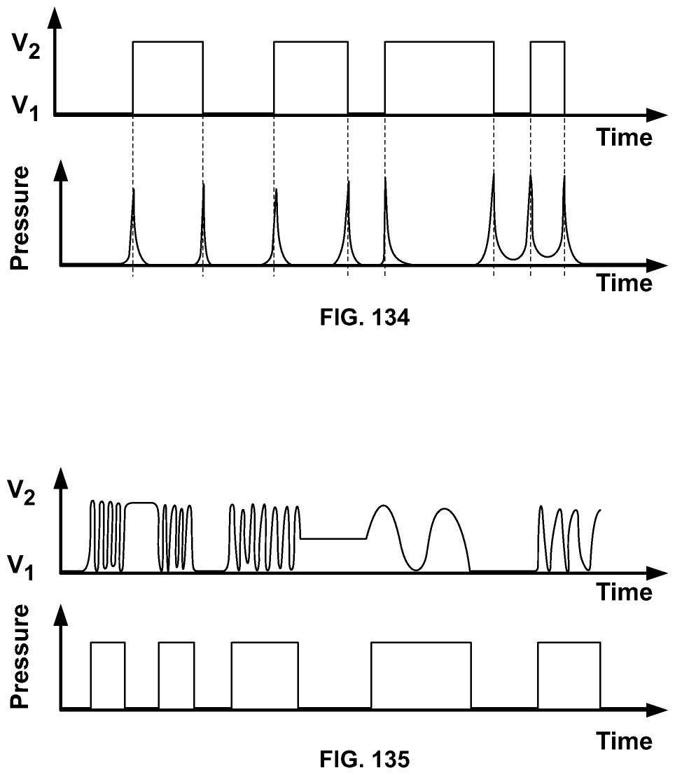

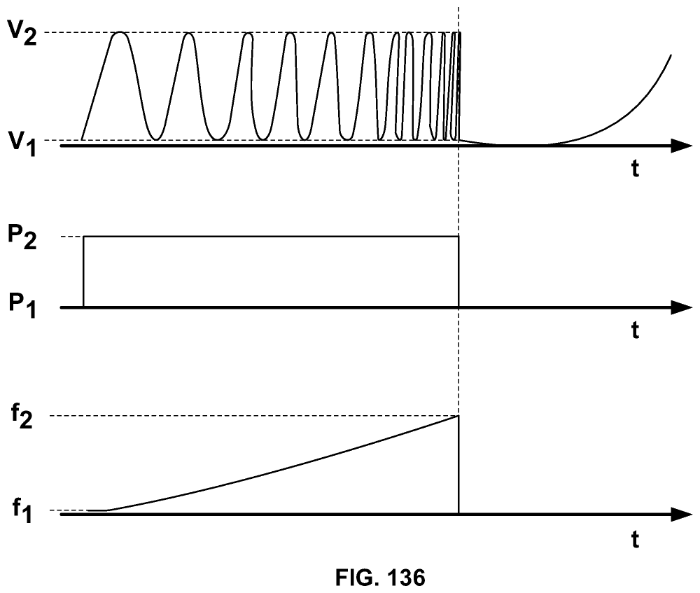

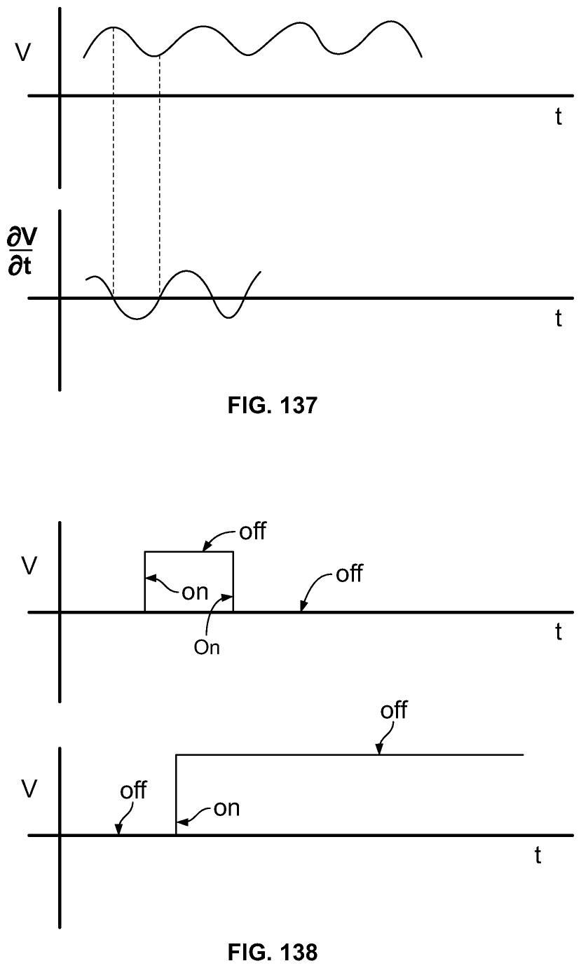

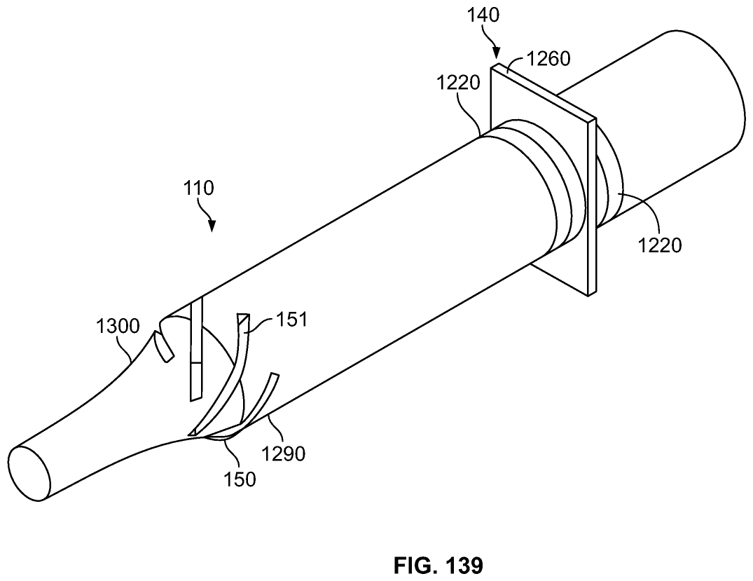

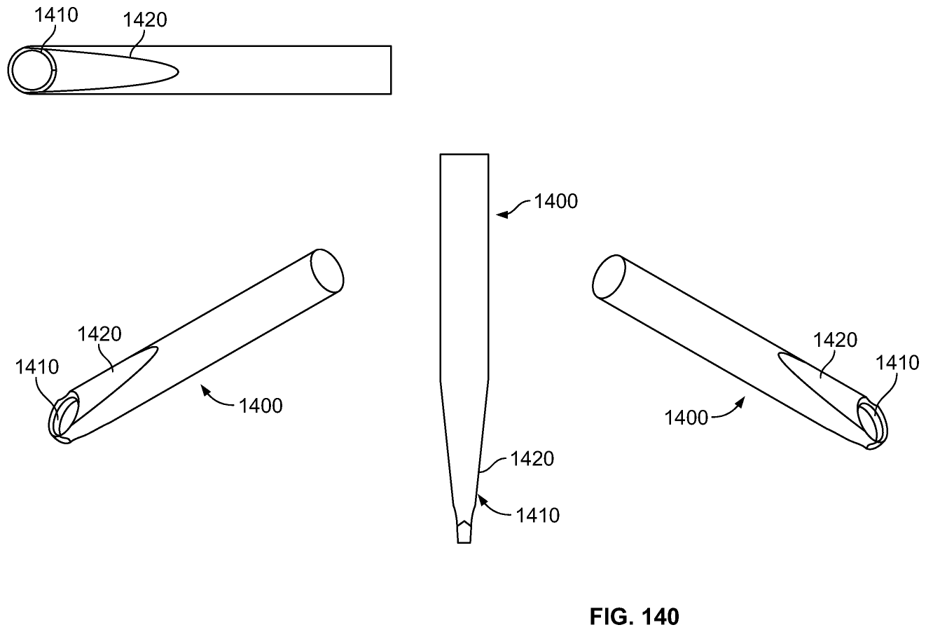

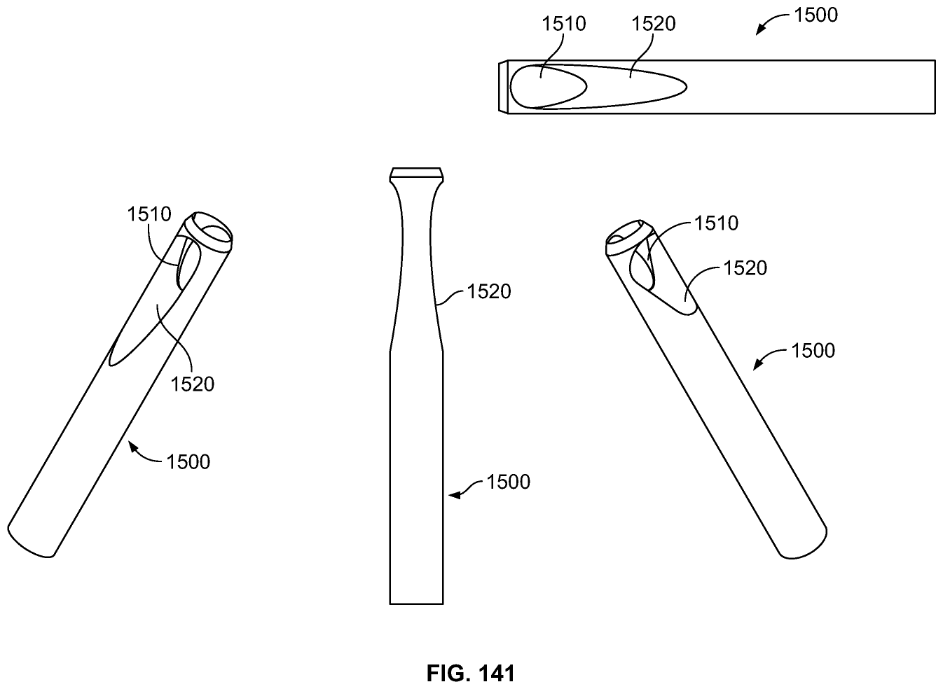

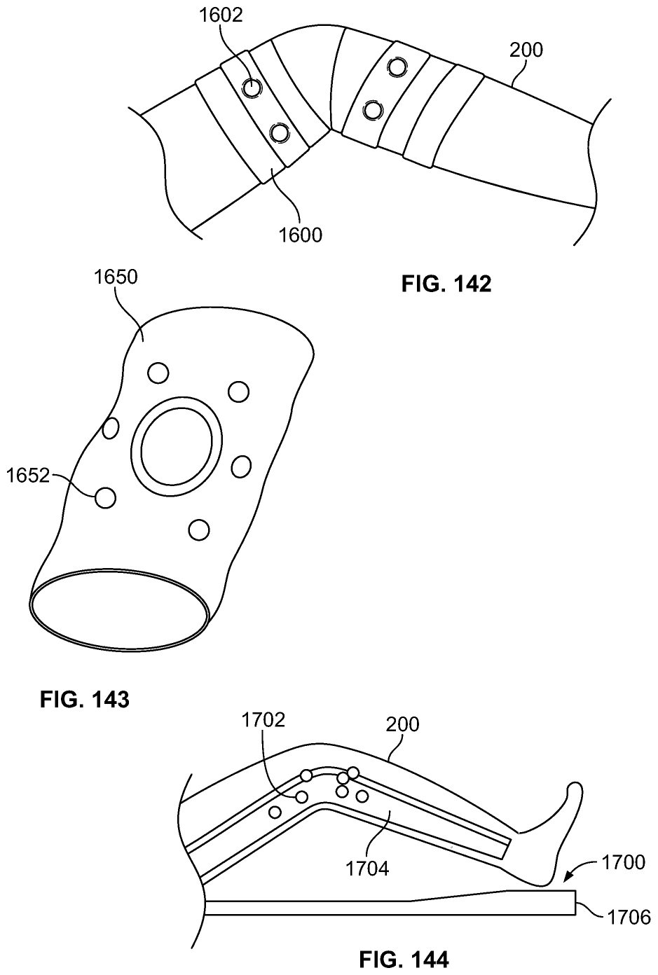

Referring to FIGS. 97-139, embodiments may include any type of effector including projectors, any type of transducer, and/or any medical or other application. Effectors and/or transducers may include directional energy application (FIGS. 97-98), a distal portion having a projector (FIG. 99-101), projector surfaces (FIGS. 102-104), capsule configurations (FIG. 105-108), illumination (FIG. 109-110), reinforcement (FIGS. 111-113), dimensional characteristics (FIG. 114-116), beam patterns (FIG. 117-119), additional capsule configurations (FIG. 120-124), anatomic specific positioning (FIG. 125), application to targeted pathologic materials and/or tissues (FIG. 126-127), transdermal configurations (FIGS. 128-129), alternate capsule configurations (FIG. 130), application to targeted tissues (FIG. 131), projector connections configured to reduce constraint and/or mass (FIG. 131-133), various wave forms (FIG. 134-138), torsional transducers (FIG. 139), distal portions (FIGS. 140-141), and applications to any medical condition (FIG. 142-144). Embodiments may further include projector 1000 (FIG. 107), capsule projector 1002 (FIG. 105), and/or ring projector 1003 (FIG. 112). Embodiments may also be configured to produce a spherical 1005 (FIG. 108) or near spherical beam pattern and/or relatively high intensity and/or frequency energy in a medium. (FIGS. 105-107, 114). The projector may be sized for minimally invasive introduction into the sinuses, for example the frontal and/or maxillary sinuses. A projector (i.e. ultrasonic or sonic projector) may be provided with energy and/or vibrate in any number of patterns and intensities to create pressure (i.e. a field of time varying pressure waves) in a portion of the body, for example the nasal cavity. Embodiments may apply energy to pathologic material 1170 (i.e. infection and/or biofilm), which may be on or integrated into the surface of the nasal cavity tissue 1168. (FIG. 126).

Projector 1003 and 400 may be mounted in a tiltable, rotatable, and/or articulating distal portion. (FIGS. 97 and 98). Cladding 1101 may cover projectors 400 and/or 1003. Cladding 1101 may include window 1110, for example, to allow a pressurized stream to flow from within projector 1100. Window 1110 may be mounted to swivel 1105 within cladding 1101. Window 1110 may be steered and/or randomly positioned to allow acoustic energy to follow the stream out of projector 1100 and/or toward the tissue and/or pathologic material 1170 at the treatment area. By moving the cladding 1101, tilt 1104, pivot 1105, and pivot 1106, the internal surface of the body cavity may be scanned with a sound field (i.e. high intensity) using the fluid stream 1102 as a wave guide. The stream 1102 may disrupt the integrity of pathologic material 1170 (i.e. biofilm), which may combine acoustic and/or physical disruption of pathologic material 1170.

Effector 110 may have various embodiments of a distal portion in medium 1005. (FIGS. 102-104). A medium may include any material that occupies a cavity and/or is near the treatment site. An embodiment may include a direction beam pattern in medium 1005 from a longitudinal effector, for example emanating from surfaces 1130 and 1131 of distal portion 1005. (FIG. 102). In another embodiment, a distal portion may include a truncated and/or conic surface 1132, for example, to more broadly spread the beam pattern in medium 1005. (FIG. 103). Alternatively, distal portion 1005 may be configured to produce a toroidal field from surface 1133. (FIG. 104). Groove may be etched or cut into the surface, for example to enhance movement with respect to medium 1005.

The effect of pressure (i.e. a time varying field of pressure) inside the nasal cavity may result in a number of responses in tissue and fluids of the nasal cavity. As an example, the maxillary sinus may have a volume of about 10 milliliters. Projector 1000, 1002, 1003, and/or 400 may have a smaller volume, for example 1 microliter. The projector may be positioned in the nasal cavity and/or energized to project sound or ultrasound, pulses, clicks, or any acoustic structure of any frequency and/or intensity, for example about 10 to 250 decibels at about 1 to 10 millimeters from projector 1000, 1002, 1003, and/or 400. The duration may be any amount of time, for example a single pulse event of a fraction of a microsecond or one hour duration of a simple wave form. (FIGS. 134-135).

Projector 1001, 1002, 1003, and/or 400 may contact the nasal tissue or may be any distance (i.e. 3-4 centimeters) from a particular location of tissue coated with pathologic material 1170. The projector energy may be transferred to medium 1005 (FIGS. 108, 124) occupying the cavity, for example the nasal cavity. The medium may include any tissue, air, or liquid. The medium may naturally reside in or be introduced into the cavity. The cavity (i.e. nasal cavity) may be the medium. The medium may include any material that occupies and/or surrounds a cavity.

Medium 1005 in the nasal cavity may be in contact with the pathologic materials (i.e. biofilm). (FIG. 108). Medium 1005 may respond to acoustic energy from the projector, for example projector 1003 (FIG. 126) and/or may excite the surface of the pathologic material 1170. The pathologic material 1170 may respond to the excitation by moving in consequence to the medium 1005. The pathologic material 1170 (i.e. biofilm) and surrounding (i.e. non-infected) structures may then transmit acoustic energy to more pathologic material 1170 (i.e. biofilm). Alternatively, the acoustic energy may be transmitted to medium 1005 then into more pathologic material 1170 (i.e. biofilm) or tissue.

The density of energy that may be transmitted from projector 1000, 1002, 1003, or 400 to pathologic material 1170 (i.e. biofilm) may be based on the acoustic matching of medium 1005 with pathologic material 1170 and/or the acoustic matching of the projector to medium 1005. The presence of a gas in the cavity (i.e. nasal cavity) may reduce the energy density.

Pathologic material 1170 (i.e. biofilm) may be radiated by an ultrasound or sound field directly or it may be affected by induced flows in the medium 1005. A projector may induce flow in medium 1005, which may cause a momentum transfer between medium 1005 and pathologic material 1170 (i.e. biofilm). (FIG. 126). At least two forms of motion, vibration and streaming flow, may work together to yield a resultant flow around and among the tissue (i.e. cilia), surrounding structures, and pathologic materials (i.e. biofilm cell structure). The motion may increase the potential for a chemical reaction between medium 1005 and pathologic material 1170. Any chemical agent or lavage introduced into the cavity (i.e. nasal cavity) may be enhanced with the motion of the medium 1005 and/or pathologic material 1170. If a solid particulate is introduced into the medium 1005, the moving medium, due to the acoustic projection, may cause the particulate to rub and/or grind against the surface microstructures of the pathologic material 1170 thereby causing them to weaken and/or separate from the tissue.

Pathologic material 1170 may be agitated after being exposed to high frequency and/or pulses of acoustic energy. (FIGS. 134, 135). The agitation may dislodge the cells from the underlying structure so that it can be washed away or the cell of the infecting bacteria may die due to the physical disturbance of the pressure field moving through the cells. (FIG. 127). The death of cell 1169 could be due to the destruction of cell 1169 microstructures and the cell may act as a wave trap and amplify by way of sympathetic resonance the local strain effects of the induced vibration. The vibration of medium 1005 may present a hostile environment for the cells of pathologic material 1170 causing a normal response in cells 1169 to release and try to find a more habitable environment free of vibration and in the process the cells 1169 may be washed away by medium 1005.

Light and other forms of electromagnetic radiation may be used to stress the cells 1169 of pathologic material 1170 and/or cause them to die off. Potentially due to shadows and penetration limitations, light alone may not be completely effective in eradicating pathologic material 1170. The use of projector 1000, 1002, 1003, and/or 400 may acts as a supplement to the electromagnetic radiation and may increase the overall effectiveness of parallel use of the radiation and sound or ultrasound. Similarly, the presence of a chemical agent, or parasite or antibiotic is enhanced by the presence of a vibratory energy field (i.e. high frequency and/or high intensity ultrasound field). A combination of light radiation, antibiotics, and ultrasound is an example of a treatment that may be effective in removing pathologic material 1170 of a body cavity, for example the nasal cavity.

An embodiment may include a handpiece configured to manipulate a distal portion of the projector to guide the distal portion through a body passage (i.e. nasal passage) and into a cavity 1167 (i.e. maxillary sinus cavity). (FIG. 125). The distal portion of the projector 1120 (FIG. 100) may be flexible and/or positionable to conform to the contours of the cavity then present projector 1000, 1002, 1003, and/or 400 to the body cavity. Distal portion 1160 may contact the tissue or may be suspended in the cavity. Medium 1005 may be introduced into the cavity with a catheter integrated into projector device 1120 or separately introduced with an irrigation catheter.

Distal portion 1160 may incorporate a light source 1162, a fluoroscopy contrast material or magnetic material to aid the navigation and placement of the device within the target cavity. (FIGS. 100, 120-124). Light source 1162 in such an embodiment may be in a distal or proximal portion of projector 1160 or projector 1003. Light source 1162 may be a light emitting diode (LED) of any color and with sufficient luminosity so that the light can be viewed through the patient's facial structures. Light 1162 may be designed to vary in intensity of color upon activation of the sound field or medium 1005 introduction or cessation thereof. Light source 1162 may be of therapeutic frequency and/or intensity.

Projector 1003 may include a piezoelectric ceramic with an electrode coating of a conductive material, for example nickel, silver, or copper. (FIG. 122). Projector 1003 may also include assembly 1002 (FIG. 105) having multiple ceramic elements, benders, and/or mounting elements forming an array. Projector 1003 may be a solid or hollow sphere, which may be an assembly of two or more hemispheres 1001. (FIG. 107). The hemispheres may be hollowed and/or bonded together. (FIG. 107).

The projector may include an assembly of one, two, three, or more piezoelectric elements and/or form a solid or hollow capsule 1002. The projector, capsule or ring, may have, in 3D, many aspect ratios such as 1:1:1 (z axis, x axis, y axis) or 10:1:1 or 1:10:10 or 1:1:2. The projector may be a ring 1003, or tube or a disk. In the case of a ring or a tube, the tube may be poled radially (FIG. 121) and the electrodes coated, one on the internal surface and one on the external surface. The tube or ring may be reinforced, for example, to enable strains in excess of the strain limits of the piezoelectric ceramic alone. (FIGS. 111-113) Reinforcement 1151 or 1152 may be in the form of a metal coating or a wound fiber exterior to increase the strength of the ring projector. It may be reinforced by a metal tube 1152 that fits over or with the piezoelectric ceramic tube 1153. The reinforcement may be in the form of a composite structure 1150 with high strength carrying materials, a binding agent such as epoxy or polyester, or any other of material suitable to from a matrix. (FIG. 113).

Projector 1103 (FIG. 120-121) may be electrically connected to conductors 1161A and 1161B with relatively small junctions configured to reduce the mass loading on the surface of the piezoelectric crystal. The junctions may be soldered, welded, cold welded, compression bonded, or gently contact with the crystal with wiskers. Projector 1001, 1002, 1003, or 400 may be supplied with energy from a generator through the conductors and the energy in is the form of a controlled voltage. The signal from the generator and the waveform is described herein. Projector 1003 may be encapsulated 1163 in enclosure 1141 (FIG. 109) or enclosure 1101 (FIG. 97). Enclosure 1141 or 1101 may be a closed sealed shell, filled with a non-conducting, and/or acoustic matching material 1163 (FIG. 124). Material 1163 may have a similar acoustic impedance as mucus or saline. Enclosure 1141 or 1101 may also include an acoustic matching material, for example polyethylene, spectrum, or urethane. This may provide the projector with an acoustic environment that may allow the acoustic energy to be efficiently transmitted through it and to pathologic material 1170 (i.e. biofilm). The enclosure 1141 or 1101 may be rigid or soft.

Other medical devices may be positioned within a capsule created by the enclosure 1141 or 1101. These devices may contribute to the function of projectors 1000, 1002, 1003, or 400 or expand the treatment possibilities. For example, the capsule may include a magnet, light 1162 (i.e. LED), or an array of lights 1142 to assist a user in navigation by providing a lighted beacon indicating distal portion 1160 or shaft of the projector 1003 enclosure 1141 or 1101.

The projector 1000 may include a hollow sphere (i.e. 3 millimeter diameter) and/or may be configured to have a substantially uniform spherical beam pattern over a broad frequency range. Embodied as a sphere and/or poled radially, the piezoelectric element(s) 1001 may strain radially, by expansion and/or contraction. Excitation of the sphere will result in spherical pressure waves propagating from the surface of the projector 1000 (FIG. 108). The sphere may have a discontinuity at a junction for the external electrode and/or a discontinuity at a penetration for a conductor to reach the inner surface of the sphere. The penetration may be anywhere on the sphere or at a seam of pieces of hemispheres constituting the sphere.

The projector in another embodiment may be a ring 1003, for example with an OD of about 2.5 millimeters, an ID of about 1.75 millimeters, and/or a length (H) of about 1.75 millimeter. This is an aspect ratio of 10:7:7. (FIG. 114). A ring with these relative dimensions will produce at least two modes of vibration over a broad frequency range. For example, a ring of this aspect ratio may possess a first fundamental breathing mode resonance between about 200 and 500 kHz depending on the material. (FIG. 115) This motion, created by expansion of the ring diameter will produce a substantially toroidal beam pattern at phase zero. (FIG. 119) Upon contraction of the ring projector, (FIG. 116), the fluid within the ring will compress and eject out of the ends of the ring yielding a 2-lobed beam 180 degrees out of phase with respect to the toroidal beam of the expansion strain. (FIG. 118) Superposition of the two modes' maxima yields a near spherical isobar around the tube. The near-spherical beam pattern may be used in hollow cavities and/or where directional access is desirable. For example, distal portion of this projector 1160 may be introduced into a curved path (i.e. 3 millimeter in length) through the maxillary cavity and into a larger cavity. (FIG. 125). Since the beam pattern is substantially spherical, the surfaces in the maxillary cavity 1165 may be exposed to direct acoustic energy. This may expose pathologic material 1170 to acoustic energy with or without reorientation of the projector 1160.

The ring projector may be configured to induce sufficient acoustic energy into the surrounding medium 1005 to cavitate medium 1005. The piezoelectric ceramic surface of projector 1000, 1002, 1003, or 400 may also be positioned close enough to medium 1005 to cavitate medium 1005. Medium 1005 may be especially susceptible to cavitation if medium 1005 is water. The ability of the fluid medium to cavitate and/or the cavitation threshold may be influenced by the hydrostatic pressure of the environment surrounding the projector. Increasing the pressure of the environment may suppress the onset of cavitation until higher energy levels are reached. As an example, at atmospheric pressure the ring projector may yield sound pressure levels in water as high as 220 decibels at the piezoelectric ceramic surface. The sound pressure level may decrease at about the inverse of 1/radius from the projector 1000. Enclosure 1163 may suppress localized cavitation onset and/or may dampen the strain of the ring, especially if the ring 1003 is excited at resonance. Running the ring projector 1003 at resonance may require less power for a given acoustic output. Depending of the strength of the piezoelectric ceramic material, the ring projector may be run at 20 volts peek-to-peek amplitude while resisting cracking the material. At this level and at resonance, cavitation may onset at as low as 6 volts peak-peak amplitude. The implication is that a small, higher frequency, resonate, dual mode, ring projector, can be powered with a battery power supply, sufficient to treat pathologic material 1170 for a period of time, for example several minutes.

In an alternative embodiment, projector 1000, 1002, 1003, or 400 may be excited at or above the cavitation threshold to agitate the medium 1005 more aggressively, to exploit the energy effect of cavitation bubble collapse to degrade the pathologic material 1170.

The projectors disclosed herein may be manufactured by any process disclosed herein or known in the art. For example, the piezoelectric element of ring projector 1003 may be an extruded material or made any other suitable process. As another example, spherical projector 1000 may be machined material or made by any other suitable process. Ring projector 1003 may be encapsulated with an index matching material 1163 in a distal portion, which may produce a high intensity acoustic field from less than 100 kHz to about 500 kHz. Other geometries may go even higher or lower in frequency.

The ring projector 1003, regardless of its aspect ratio, may enable ease of mounting with distal portion 1160, 1140 and 1120. The conductors may be connected without the need to drill penetrations through the piezoelectric ceramic. The ring projector 1003 is particularly suited to ease auxiliary device placement distal to the ring since the conductors of attachments may be routed through the internal passage.

The projector 1003 may not be a dual mode ring projector as shown in figures (FIG. 118 119). It may simply act as a cylinder and produce a cylindrical beam pattern. This would be the case if the aspect ratio of the ring is about 10:7:8 (OD:ID:Length (H)) (FIG. 114) with the length (H) greater than 7 as high as 100.

The projector distal portion encapsulation 1163 must be flexibly affixed to the device 1164 (FIG. 124) and the piezoelectric ceramic 1003 may be flexibly affixed to the encapsulation 1163. A rigid attachment will severely inhibit acoustic output or cause the output to be non-uniform. In an additional embodiment, a rigid attachment may be applied at a node of vibration in higher modes of vibration. (not shown) This is a means to enable rigid attachment if need be.

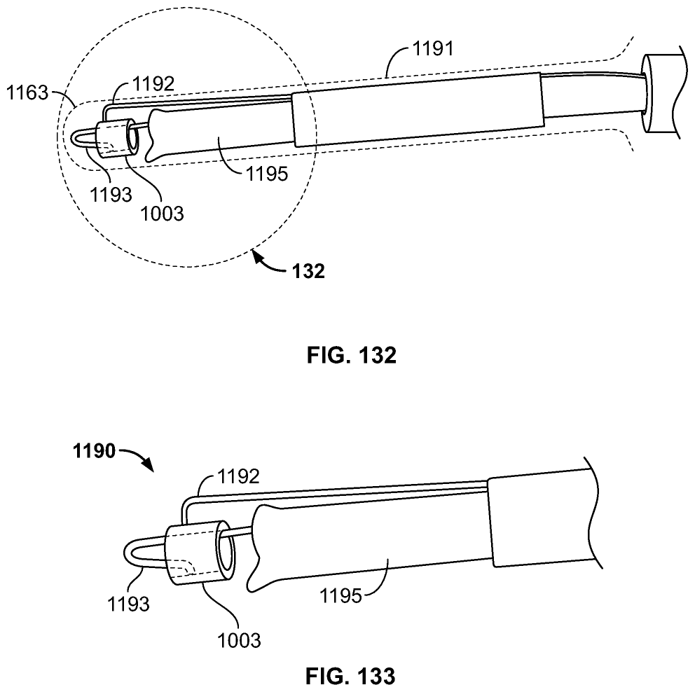

A net or tin fabric may be integral to distal portion 1160 or the encapsulation 1163 to enable an attachment to the device 1120 for example where the net or web extend out of the encapsulation. The net or web may be metal, plastic, silicone, or any material suitable for contact with the body. Alternatively, the projector 1003 may be suspended in a balloon 1191 (FIG. 132) filled with an index matching fluid 1163, such as mineral oil or deionized water. The balloon 1191 material may be polyethylene or silicone or urethane or some other compliant material that has an acoustic impedance similar to water or mucus. The projector 1103 is flexibly attached by light conductive springs 1193 and 1192 that contact the inner and outer electrodes of the radially poled projector 1003. Alternatively, the projector may be flexibly supported by an insert 1122 (FIG. 99)

The balloon device 1190 may be housed within a temporary enclosure to facilitate navigation into the various sinus cavities. The temporary enclosure may be retracted so as to deploy the projector 1190 into the nasal cavity.

An additional embodiment allows the balloon 1191 to be fully inflated in a compliant manner to fill the nasal cavity 1165 with a medium 1005, but contained within the boundaries of the balloon 1191. The pathologic material 1170 will then be radiated with acoustic energy through the balloon material.

In this embodiment, the hydrostatic pressure within the balloon 1191 may be elevated to suppress the onset of cavitation. The balloon 1191 may be given a post inflation shape so as to reach into the contours of the nasal cavity 1165 upon inflation. The 1190 may be constructed with a fill catheter and a check valve to prevent the index-matching fluid to migrate back into the reservoir.

The projector 1103 may be placed within the handpiece in another embodiment, with the aforementioned medium 1005 or 1163 filled balloon 1191 acting as a wave guide to carry the acoustic energy into the nasal cavity. In such an embodiment an impedance mismatching tube may be fitted external to portions of the balloon to shield the balloon from the body or air, thereby aiding in containing the acoustic energy within the index-matching fluid 1163.

Array 1180 may include piezoelectric ceramic crystals 1181 positioned in an array. Array 1180 may be configured to conform and/or be removably affixed to an internal or external surface of the body. An external surface may include skin, for example overlying bones of a cheek. (FIGS. 128-129). Crystals 1181 may include a patch with an adhesive 1182 coated over the crystals and/or may provide index matching to the body surface, for example the skin. Crystal 1181 may be energized by the generator and/or vibratory energy may be projected from array 1180, for example, into the body. Vibratory energy may be projected to pathologic material 1170.

Transformer 150 includes one or more slit 151 that may enable effector 110 to apportion at least some of the longitudinal acoustic waves from the vibratory energy into torsional and/or transverse waves. (FIG. 138). Slit 151 may extend generally helically about the longitudinal axis of effector 110. Slit 151 may span at least a segment of first portion 1290 and at least a segment of second portion 1300. Alternatively, slit 151 may extend in any orientation and/or location that enables effector 110 to function as described herein, for example. For example, slit 151 may be straight, curvilinear, or irregular. Slit 151 may be closed such that slit 151 is defined internally and is not exposed at the surface. Alternatively, slit 151 may be any size and/or shape that enables transducer assembly 200 to function as described herein, for example having a width of at least about 0.001″. A material of higher or lower density and/or modulus of elasticity than a material used to fabricate effector 110 (i.e. aluminum 6061-T6) may be positioned within slit 151 to facilitate altering the longitudinal vibratory energy.

Distal portions 1400 and 1500 may be coupleable to the distal end of effector 110. Vibratory energy may be transmitted through effector 110 to distal portion 1400 and/or 1500. (FIGS. 130-141). Distal portions 1400 and/or 1500 may be configured to transmit longitudinal and/or non-longitudinal vibratory energy to the treatment area to facilitate cutting and/or emulsifying a material at the treatment site. In the exemplary embodiment, distal portion 1400 and/or 1500 may include at least one cutting edge 1410 that is configured to cut and/or emulsify material and flute 1420 that is sized and/or configured to capture the cut and/or emulsified material therein. Distal portion 1400 and/or 500 may be configured to enable a user to yield an uninterrupted strand of cut tissue. Cutting edge 410 may have an aspect ratio between about 20:1 (i.e. width of 20:height of 1) to 1:100 (i.e. width of 1:height of 100). As shown in FIG. 140, the aspect ratio of cutting edge 1410 may be 1:1. Distal portion 400 and/or 500 may be configured to vibrate in a plane that is substantially normal to cutting edge 1410. Distal portion 400 and/or 500 may be configured to vibrate in any plane that enables effector 110 to function as described herein. Embodiments of the present disclosure may also include any devices and methods disclosed in U.S. Provisional Application No. 61/677,776, titled “Methods and Systems for Extracting Tissue using a Handheld Medical Device”, which is hereby incorporated by reference in its entirety.

Embodiments may include one or more transducers integrated into other medical devices. (FIGS. 142-144). Transducers may be integrated into wound dressings, braces, continuous passive motion (CPM) devices, blood pressure cuffs, heating pads, surgical drapes, gowns, tourniquet, and compression socks. For example, wound dressing 1600 may include one or more transducers 1602 to remove or resist formation of pathologic material, for example biofilm or infection. (FIG. 142). The ultrasound waveform could be modulated to alternate between biofilm treatment and functioning as wound debrider.

Another embodiment may include transducers 1652 in joint brace or wrap 1650. (FIG. 143). Transducers 1652 may be configured to treat biofilm infections as a protocol, which may be prescribed for the patient to optimize treatment. For example, the patient might be prescribed to wear brace or wrap 1650 for the three thirty minute sessions each day.

As further embodiment, transducers 1702 may be integrated into a continuous passive motion device (CPM). (FIG. 144) CPM 1700 may be used following joint construction surgery to control pain and/or reduce inflammation. By including transducers 1702 in cuffing 1704 and/or platform 1706. Transducers 1702 may be configured to target vibratory energy at a joint replacement and/or biofilm infections. Biofilm may be reduced, resisted, and/or prevented. The transducers could also be located in the straps on CPM 1700. The transducers could also be integrated into other exercise and rehabilitation devices. The ultrasound waveform could be modulated to alternate between biofilm treatment and functioning as a low intensity ultrasound bone growth stimulator.

The embodiments described herein may be used on or for treatment of any other portion of a body. Embodiments herein may be autoclavable and/or sterilizable. Embodiments may be used in a blood vessel or a cardiovascular region of the body. Further embodiments may be used on a lung, tumor, polyp, urethra, ureter, bladder, kidney, gallstone, gallbladder, clots, esophagus, bile duct, asthma, nasal passage, abdomen, ear (i.e. ear wax), prostate, gastrointestinal tract, liver, bronchus, trachea, eustachian tube, arteries, veins, and/or intestines. Pathologic materials (i.e. calcific deposits) may be removed from any portion of the body.

Embodiments may be configured for an ear, for example to treat otitis media. Otitis media may include inflammation of the middle ear, between the tympanic membrane and the inner ear, including a duct known as the eustachian tube. The effector may include an elongated portion and/or be configured to penetrate and/or drain pathologic material, for example a clog, blockage, infection, and/or biofilm in or around the eustachian tube. The effector may be positioned percutaneously through the tympanic membrane or directly into the eustachian tubes to loosen, drain, and/or clear pathologic material. The effector may also be configured for transcutaneous energy application through the skin.

Another embodiment may be configured for a colon, for example to treat diverticulitis. Another area to use this is diverticulitis of the colon or if patients have impaction. Chronic consdistal portionation, impaction, or different types of blockages may be transcutaneously or percutaneously treated. For example, embodiments may include an effector to enlarge or open a colon, esophagus, and/or intestines.

Further embodiments may be configured for a prostate, for example to treat prostatitis and/or benign prostatic hypertrophy (BPH). Inflammation or irritation of the prostate may result from, for example, bacteria, injury, kidney stones, or cancer. The prostate may narrow due to thickening of the membranes, lack of secretions, or hypertrophy of the prostate. To treat these conditions, an effector (i.e. wire shaped) may include an elongate member and/or be positioned though a catheter (i.e. Foley catheter) into the prostate with or without an imaging device. The effector may also be positioned percutaneously through the urethra. Energy may be applied to the effector to remove pathologic material from and/or increase, widen, shrink, and/or heat the passage, for example, of the urethra and/or prostate. This could be used to widen the prostate. Application of vibratory energy to the effector may drive the effector in a toroidal and/or circular fashion with respect to the passage to widen and/or enlarge the passage. This may potentially reduce unnecessary tissue removal, bleeding, discomfort, medications, and/or invasive surgeries.

Embodiments may also be configured for the ureters and/or kidneys, for example to treat kidney stones, nephrolithiasis, or a condition including calcification. For example, if there is severe calcification around the kidney, the effector may selectively separate calcium from the kidney tissue by applying energy based on material characteristics (i.e. modulus of elasticity). Calcified tissue may be broken up and remove. In use, an effector may be positioned into and/or through the vessels (i.e. ureters) and kidneys. The effector may apply energy to reduce plaque or calcifications in the vessels and/or kidneys. Effector may also be configured to percutaneously or transcutaneously remove all or portions of the kidney, for example, when the kidney calcifies. The kidney may calcify with recurrent kidney stones that obstruct the kidney and/or the entire of the urethral system. The effector may include an elongated portion and/or be positioned around the kidney or the skin adjacent the kidney to apply energy to reduce and/or release pathologic material (i.e. calcified or deposits) in the kidney.

Additional embodiments may be configured for a lungs, pancreas, liver, and/or intestine, for example, to reduce cystic fibrosis, recurrent infections, and/or other pathologic material. Cystic fibrosis may include scarring and cyst formation. The effector may be positioned in and/or energy may be applied to the lungs, for example, the bronchials and bronchi.

Embodiments may be configured for a bile duct, gallbladder, and/or cholecystitis. An effector may be positioned in and/or apply energy to calcific deposits in the bile duct and/or gallbladder. The effector, for example including an elongated portion, may be positioned in the bile duct and/or gallbladder. Also, effector 110 used for endoscopic retrograde cholangiopancreatography (ERCP), for example, to widen the gallbladder and/or remove calcific deposits in the gallbladder and/or bile duct. The effector may be positioned in and remove calcification, tissue, gallstones, or other materials that may be blocking the bile duct or gallbladder, which may avoid a cholecystectomy and/or removal of the gallbladder and/or the bile duct. The effector may be positioned retrograde through ERCP with an imaging or guidance device, for example endoscopic guidance and/or a combination of endoscopic radiographic guidance. This may increase and/or open the passage of the gallbladder and/or biliary tree and/or remove the gallstones.

Further embodiments may be configured for chondrocalcinosis of a joint, for example a knee joint. Pathologic materials (i.e. calcium crystals) may coat the articular surface or other surfaces of a joint. Instead of removing the cartilage surface along with the pathologic materials, the effector may apply energy transcutaneously through the skin to the pathologic materials, percutaneously through a passage, and/or to fluid in the joint (i.e. synovial fluid) to reduce pathologic materials. This may reduce further inflammation, damage, and/or arthritis.

Additional embodiments may be configured for cranial surgery. For example, if a brain has hydrocephalus (i.e. normal pressure hydrocephalus) and/or limited drainage, an effector may be positioned in a ventricle to widen a drainage passage and/or reduce the pressure. This procedure may be used in conjunction with an imaging device, for example MRI or x-ray. Also, it may be used in conjunction with or replace a shunt.

Embodiments may also be configured for myositis ossificans or heterotopic ossification, for example when muscle or tendons become calcified from trauma or genetic predispositions. The tissue may be calcified and/or movement and function may be lost. By applying energy, pathologic material (i.e. calcified tissue) may be separated from the normal or healthy tissue, for example the underlying bone, tendon, ligaments, or muscles. The calcium may be removed from these tissues leaving the viable muscle, which can work and function. If the muscle is calcified, essentially, it is stiff and does not work again. This can occur after hip and knee arthroplasty, trauma, or injury. Vibratory energy may be utilized to selectively remove the pathologic material (i.e. calcified tissue) from the normal tissue. Muscle, ligaments, and/or tendons may regain function and become elastic and/or pliable. This may be used for infectious tissue or to debride devitalized tissue from normal tissue,. Energy may be configured for removing scar tissue from normal tissue, which may be more pliable than rigid scar tissue. Pathologic materials may be differentially separated in order to loosen and/or remove the scar tissue. Pulsed irrigation, lavage, saline, gelatin and/or any material disclosed herein may remove this from the body tissue. Embodiments may be used with normal saline or other fluids that allow transmission of the ultrasound. This may be used percutaneously, transcutaneously, or on multiple separate treatment areas, for example, including a single treatment or multiple treatments.

Another embodiment may be configured for a colon, for example to treat diverticulitis. Another area to use this is diverticulitis of the colon or if patients have impaction. Chronic consdistal portionation, impaction, or different types of blockages may be transcutaneously or percutaneously treated. For example, embodiments may include an effector to enlarge or open a colon, esophagus, and/or intestines.

Other medical devices may benefit from vibratory energy (i.e. ultrasonic energy), for example, to cauterize bleeding and/or position implants. Embodiments may use energy to bond, assemble, secure, and/or position implants. Energy may be applied preoperatively, intraoperatively, or postoperatively. For example, vibratory energy may be utilized to bond tissue fasteners and/or joint replacements with respect to a portion of the body. Vibratory energy may be utilized to bond multiple components of the embodiments herein with respect to each other. Embodiments of the present disclosure may include any additional devices and methods disclosed in any of U.S. Pat. No. 7,967,820, titled “Methods and Devices for Trauma Welding” and U.S. Pat. No. 8,162,977, titled “Methods for Joining Implants” and U.S. Patent Application Publication Nos. 2009/0024161, titled “Methods and Devices for Intracorporeal Bonding of Implants with Thermal Energy”, 2010/0211120, titled “Methods and Devices for Utilizing Bondable Materials”, 2012/0316472, titled “Ultrasonic Handpiece”, 2012/0316473, titled “Methods and Systems for Controlling an Ultrasonic Handpiece based on a Sensed Pressure”, and 2012/0316474, “Methods and Systems for Controlling an Ultrasonic Handpiece based on Tuning Signals”, all of which are hereby incorporated by reference in their entirety.

In addition, embodiments of the present disclosure may include all or any portion of the embodiments disclosed in U.S. Pat. No. 7,837,736, titled “Minimally Invasive Surgical Systems and Methods” and U.S. Patent Application Publication No. 2008/0069855, titled “Method of Inhibiting the Formation of Adhesions and Scar Tissue and Reducing Blood Loss”, all of which are incorporated herein by reference in their entirety.

As used herein, an element or step recited in the singular and proceeded with the word “a” or “an” should be understood as not excluding plural elements or steps unless such exclusion is explicitly recited. Furthermore, references to “one embodiment” of the present disclosure or the “exemplary embodiment” are not intended to be interpreted as excluding the existence of additional embodiments that also incorporate the recited features.

This written description uses examples to disclose various embodiments, which include the best mode, to enable any person skilled in the art to practice those embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system for applying vibratory energy to pathologic material in a treatment area of a body, the system comprising:
an energy source configured to provide an energy signal;
a piezoelectric transducer configured to receive the energy signal;
an effector operatively coupled to the transducer, the effector having a proximal end connected to the handle and a distal portion configured to apply vibratory energy to pathologic material; and
a cannula having a longitudinal passage to receive at least a portion of the effector and being configured to expose at least the distal portion of the effector to the pathologic material,
wherein the transducer is configured to transfer vibratory energy through the effector to the pathologic material.

2. The system of claim 1, further comprising a handle connected to the energy source, wherein the transducer is disposed in the handle.

3. The system of claim 1, wherein the transducer is positioned at the distal portion of the effector.

4. The system of claim 1, wherein the pathologic material is disrupted by vibratory energy applied transdermally a distance from the pathologic material.

5. The system of claim 1, wherein the distal portion of effector contacts the pathologic material.

6. The system of claim 1, wherein the vibratory energy includes acoustic energy emanating a distance from the distal portion of the effector.

7. The system of claim 1, wherein the treatment area includes a passage or cavity.

8. The system of claim 1, wherein the treatment area includes a sinus cavity.

9. The system of claim 8, wherein the effector applies vibratory energy from in the sinus cavity.

10. The system of claim 1, wherein the pathologic material include mucus.

11. The system of claim 1, wherein the pathologic material include biofilm.

12. The system of claim 1, wherein the energy signal is based on a material characteristic of the pathologic material or the treatment area.

13. The system of claim 1, further comprising a pressure source configured to apply positive or negative pressure to the longitudinal passage of the cannula.

14. The system of claim 1, wherein the cannula includes a port configured to remove substances from or inject materials into the longitudinal passage of the cannula.

15. A system for applying vibratory energy to pathologic material in a treatment area of a body, the system comprising:
a handle connected to an energy source configured to provide an energy signal;
a piezoelectric transducer configured to receive the energy signal;
an effector operatively coupled to the transducer, the effector having a proximal end connected to the handle and a distal portion configured to apply vibratory energy to pathologic material; and
wherein the transducer is configured to transfer vibratory energy through the effector.

16. The system of claim 15, wherein the transducer is disposed in the handle.

17. The system of claim 15, wherein the transducer is positioned at the distal portion of the effector.

18. The system of claim 15, wherein the pathologic material is disrupted by vibratory energy transdermally applied a distance from the pathologic material.

19. The system of claim 15, wherein the distal portion of effector contacts the pathologic material.

20. The system of claim 15, wherein the vibratory energy includes acoustic energy emanating a distance from the distal portion of the effector.

21. The system of claim 15, wherein the treatment area includes a passage or cavity.

22. The system of claim 15, wherein the treatment area includes a sinus cavity.

23. The system of claim 22, wherein the effector is positionable in the sinus cavity and applies vibratory energy from in the sinus cavity.

24. The system of claim 15, wherein the pathologic material include mucus.

26. The system of claim 15, wherein the pathologic material include biofilm.

27. The system of claim 15, wherein the energy signal is based on a material characteristic of the pathologic material or the treatment area.

28. A method for using a vibratory energy system in a treatment area of a body, the method comprising:
providing a handpiece having a piezoelectric transducer operatively connected to a effector;
providing a cannula having a longitudinal passage configured to receive the effector and being configured to shield a portion of the body from the effector;
positioning the effector and cannula in the treatment area with at least a distal portion of the effector extending beyond the cannula; and
applying energy to the transducer to vibrate the effector;
wherein the effector is configured to transfer vibratory energy to the treatment area.

29. The method of claim 28, wherein the treatment area includes a passage or cavity.

30. The method of claim 28, wherein the treatment area includes a sinus cavity.

30. The method of claim 28, wherein the vibratory energy disrupts pathologic material at or near the treatment area.

31. The method of claim 30, wherein the positioning act includes passing the distal portion of the effector through a nostril of the body and into the sinus cavity.

32. The method of claim 30, wherein the positioning act includes passing the distal portion of the effector through a palate of a mouth and into the sinus cavity.

33. A method for using a vibratory energy device in a treatment area of a body, the method comprising:
providing a handpiece including an effector having a flexible elongated portion and a distal portion;
forming a hole through an imperforate tissue of the body;
passing at least the distal portion of the effector through the hole and into the treatment area; and
applying vibratory energy to the treatment area with at least the distal portion of the effector.

34. The method of claim 33, wherein the hole is in a palate of a mouth.

35. The method of claim 33, wherein the treatment area includes a sinus cavity.

36. The method of claim 33, wherein the hole is formed with vibratory energy from the distal portion of the effector.

37. A method of controlling a vibratory energy device, the method comprising:
applying a first tuning signal in a first desired frequency range to a vibratory energy device;
receiving a returned parameter from the vibratory energy device;
detecting two or more operating frequencies from the returned parameter; and
modulating a drive signal to the vibratory energy device between detected operating frequencies in a pattern until two are more detected frequencies have been applied.

38. The method of claim 37, wherein the pattern is pseudorandom.

39. The method of claim 37, further comprising the act of applying a second tuning signal in a second desired frequency range to the vibratory energy device.

40. The method of claim 37, wherein the drive signal is based on a material characteristic of a tissue of a body.

41. The method of claim 39, wherein the drive signal is based on a material characteristic of a pathologic material.

42. A system for applying vibratory energy to pathologic material in a treatment area of a body, the system comprising:
a medical device positionable external to a portion of the body; and
an effector including a piezoelectric transducer disposed in the medical device,
wherein the effector configured to apply energy to a portion of the body.

43. The system of claim 42, wherein the transducer is configured to remove a pathologic material from the portion of the body.

44. The system of claim 43, wherein the pathologic material includes biofilm.

45. The system of claim 44, wherein application of energy to biofilm causes a reduction in biofilm in the treatment area.

45. The system of claim 42, wherein the transducer is configured to image a portion of the body.

46. The system of claim 1, wherein the effector includes an abrasive contact surface configured to erode pathologic material.

47. The system of claim 46, wherein erosion of pathologic material by the abrasive contact surface is initiated by a movement pattern including superposition of two or more waveforms that is induced by the energy signal.

48. The system of claim 47, wherein the effector moves while an acoustic medium or the treatment area is irrigated.
